(19) United States
(12) Patent Application Publication
(10) Pub. No.: US 2005/0256164 A1
(43) Pub. Date: Nov. 17, 2005

(54) NK1 and NK3 antagonists

(75) Inventors: Brian T. O'Neill, Haddam, CT (US); Vinod D. Parikh, Mystic, CT (US); Willard M. Welch JR., Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/123,331

(22) Filed: May 6, 2005

Related U.S. Application Data
(60) Provisional application No. 60/570,508, filed on May 12, 2004.

(51) Int. Cl. C07D 211/68; A61K 31/445; C07D 41/02; A61K 31/4545

(52) U.S. Cl. 514/317; 514/318; 546/194; 546/226

(57) ABSTRACT

The invention is to a compound exhibiting neurokinin inhibitory properties, a pharmaceutical composition comprising same and a method of treatment for neurokinin-mediated conditions.

### FIELD OF THE INVENTION

The invention pertains to compounds which are antagonists to tachykinins, including substance P and other neurokinins (NK); to pharmaceutical compositions comprising the same; and methods of treating neurokinin-mediated diseases, among others.

### BACKGROUND OF THE INVENTION

The mammalian peptide Neurokinin B (NKB) belongs to the Tachykinin (TK) peptide family which also includes Substance P (SP) and Neurokinin A (NKA). Pharmacological and molecular biological evidence has shown the existence of three subtypes of TK receptor (NK-1, NK-2 and NK-3). Substance P (also known as NK-1) is a naturally occurring undecapeptide so named due to its prompt stimulatory action on smooth muscle tissue. More specifically, substance P is a pharmacologically active neuropeptide produced in mammals and possessing a characteristic amino acid sequence as illustrated in U.S. Pat. No. 4,680,283. Selective peptidic NK-3 receptor antagonists are also known (Drapeau, 1990 Regul. Pept., 31, 125-135). NK-1 antagonists have been previously reported in EP528495A1.

### SUMMARY OF THE INVENTION

In one practice, the invention relates to a compound having Formula I: 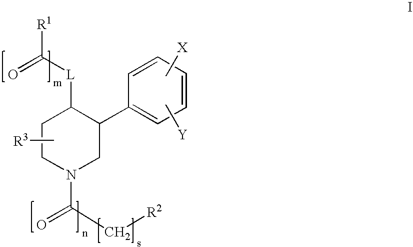
or pharmaceutically acceptable salts or solvates thereof wherein:

m=0 or 1;

n=0 or 1;

s=0 or 1;

L is —O— or —N(R⁴)—;

R¹ and R² are each independently H, aryl, heteroaryl, (C₁-C₆)alkyl, heterocyloalkyl, —(C₁-C₆)alkylheterocycloalkyl, —(C₁-C₆)alkylheteroaryl, —(C₁-C₆)alkyl-O-aryl, —(C₁-C₆)alkylaryl, and —CH₂N(R⁴)(R⁵), wherein each of said heterocyloalkyl, —(C₁-C₆)alkylheterocycloalkyl, —(C₁-C₆)alkylheteroaryl, —(C₁-C₆)alkyl-O-aryl, aryl, —(C₁-C₆)alkylaryl, heteroaryl, and —CH₂N(R⁴)(R⁵), is optionally substituted with 1-3 moieties independently selected from X′, Y′ or Z′;

R³ is H, CF₃, OH, or —(C₁-C₆)alkyl;

R⁴, and R⁵, are each independently selected from H, —(C₁-C₆)alkyl, or —(C₁-C₆)(C═O)R⁷;

R⁷ is (C₁-C₆)alkyl, OH, —N(R⁴)(R⁵), or —OR⁴;

R⁸ and R⁹ are each independently (C₁-C₆)alkyl;

X, Y, X′, Y′ and Z′ are each independently selected from H, —(C₁-C₆)alkyl, —(C₁-C₆)alkyl-NR⁴R⁵, CF₃, OH, —O—(C₁-C₆)alkyl, —(C₁-C₆)alkyl-C(═O)R⁷, aryl, heteroaryl, cycloalkyl, —NO₂, —(C₁-C₆)alkylaryl, —O-aryl, halogen, CN, —CH₃N(R⁴)(R⁵), —C(═O)R⁷, —C(═O)R⁷, —R⁶C(═O)R⁷ or —R⁶C(═O)NR⁴R⁵; and

R⁶ is a bond, —CH₂—, —O—, or —NR⁴—.

Another practice of the invention relates to a pharmaceutical composition for antagonizing the effect of NK-1 and/or NK-3 at their receptor sites in a mammal, including a human, comprising an NK-1 and/or NK-3 receptor antagonizing amount of a compound of Formula I, or a pharmaceutically acceptable salt or solvate thereof, and a pharmaceutically acceptable carrier.

Another practice of the invention relates to a pharmaceutical composition for treating a condition or disorder associated with the activity, preferably the overactivity, of NK-1 and/or NK-3 receptors in a mammal, including a human, comprising an amount of a compound of Formula I, or a pharmaceutically acceptable salt or solvate thereof, and a pharmaceutically acceptable carrier, wherein the amount of said compound of Formula I is effective in (1) antagonizing the NK-1 and/or NK-3 receptor, and/or (2) treating said condition or disorder. The “activity” of NK-1 and/or NK-3 receptors refers to overactivity, underactivity or normal activity of these receptors.

Another practice of the invention relates to a pharmaceutical composition for treating in a mammal, including a human, a condition or disorder selected from the group consisting of sleep disorders, autism, pervasive development disorder, rheumatoid arthritis, osteoarthritis, fibromyalgia, human immunodeficiency virus (HIV) infections, dissociative disorders, anorexia, bulimia; ulcerative colitis, Crohn's disease, irritable bowel syndrome, functional abdominal pain, chronic fatigue syndrome, sudden infant death syndrome (SIDS), overactive bladder, chronic cystitis, chemotherapy induced cystitis, cough, angiotensin converting enzyme (ACE) induced cough, itch, hiccups, premenstrual syndrome, premenstrual dysphoric disorder, schizophrenia, schizoaffective disorder, delusional disorder, substance-induced psychotic disorder, brief psychotic disorder, shared psychotic disorder, psychotic disorder due to a general medical condition, schizophreniform disorder, amenorrheic disorders such as desmenorrhea, obesity, epilepsy, primary movement disorders, spasticities, Scott's syndrome, Tourette's syndrome, palsys, amyolateral sclerosis (ALS), akinetic-rigid disorders, akinesias, dyskinesias, restless leg syndrome, movement disorders associated with Parkinson's disease or Huntington's disease, mastalgia syndromes, motion sickness, immune dysfunctions, generalized anxiety disorder, panic disorder, social phobia, agoraphobia, specific phobias, obsessive-compulsive disorder, post-traumatic stress disorder, major depression, single episode depression, recurrent depression, child abuse induced depression, postpartum depression, dysthemia, cyclothymia, bipolar disorder, neurocardiac syncope, neurogenic syncope, hypersensitive Carotid sinus, neurovascular syndrome, arrythmias, addiction disorders involving addictions to behaviors, HIV-1 associated dementia, AIDS dementia complex, HIV encephalopathy, HIV related neuralgias, AIDS related neuralgias, epilepsy, attention deficit hyperactivity disorder, a somatoform disorder selected from the group consisting of somitization disorder, hypochondriasis, somatoform pain disorder and undifferentiated somatoform disorder, and somatic symptoms selected from the group consisting of loss of appetite, insomnia, interrupted sleep, early morning awakening, tired awakening, loss of libido, restlessness, fatigue, constipation, dyspepsia, heart palpitations, headache, neck pain, back pain, limb pain, joint pain, abdominal pain, dizziness, nausea, heartburn, nervousness, tremors, burning and tingling sensations, morning stiffness, abdominal pain, abdominal distention, gurgling, diarrhea, and the symptoms associated with generalized anxiety disorder, comprising an amount of a compound of Formula I, or a pharmaceutically acceptable salt or solvate thereof, and a pharmaceutically acceptable carrier, wherein the amount of said compound of Formula I is effective in (1) antagonizing an NK-1 and/or NK-3 receptor, and/or (2) treating said condition or disorder.

Another practice of the invention relates to a method of antagonizing an NK-1 or NK-3 receptor in a mammal, including a human, comprising administering to said mammal an NK-1 or NK-3 antagonizing amount of a compound of Formula I, or a pharmaceutically acceptable salt or solvate thereof.

Another practice of the invention relates to a method of treating a condition or disorder associated with the activity, preferably the overactivity, of NK-1 and/or NK-3 receptors in a mammal, including a human, comprising administering to said mammal, including a human, in need of said treatment an amount of a compound of Formula I, or a pharmaceutically acceptable salt or solvate thereof, wherein the amount of said compound of Formula I is effective in (1) antagonizing the NK-1 and/or NK-3 receptor, and/or (2) treating said condition or disorder.

Another practice of the invention relates to a method of treating in a mammal, including a human, a condition or disorder selected from the group consisting of sleep disorders, autism, pervasive development disorder, rheumatoid arthritis, osteoarthritis, fibromyalgia, human immunodeficiency virus (HIV) infections, dissociative disorders, anorexia, bulimia; ulcerative colitis, Crohn's disease, irritable bowel syndrome, functional abdominal pain, chronic fatigue syndrome, sudden infant death syndrome (SIDS), overactive bladder, chronic cystitis, chemotherapy induced cystitis, cough, angiotensin converting enzyme (ACE) induced cough, itch, hiccups, premenstrual syndrome, premenstrual dysphoric disorder, schizophrenia, schizoaffective disorder, delusional disorder, substance-induced psychotic disorder, brief psychotic disorder, shared psychotic disorder, psychotic disorder due to a general medical condition, schizophreniform disorder, amenorrheic disorders such as desmenorrhea, obesity, epilepsy, primary movement disorders, spasticities, Scott's syndrome, Tourette's syndrome, palsys, amyolateral sclerosis (ALS), akinetic-rigid disorders, akinesias, dyskinesias, restless leg syndrome, movement disorders associated with Parkinson's disease or Huntington's disease, mastalgia syndromes, motion sickness, immune dysfunctions, generalized anxiety disorder, panic disorder, social phobia, agoraphobia, specific phobias, obsessive-compulsive disorder, post-traumatic stress disorder, major depression, single episode depression, recurrent depression, child abuse induced depression, postpartum depression, dysthemia, cyclothymia, bipolar disorder, neurocardiac syncope, neurogenic syncope, hypersensitive Carotid sinus, neurovascular syndrome, arrythmias, addiction disorders involving addictions to behaviors, HIV-1 associated dementia, AIDS dementia complex, HIV encephalopathy, HIV related neuralgias, AIDS related neuralgias, epilepsy, attention deficit hyperactivity disorder, a somatoform disorder selected from the group consisting of somitization disorder, hypochondriasis, somatoform pain disorder and undifferentiated somatoform disorder, and somatic symptoms selected from the group consisting of loss of appetite, insomnia, interrupted sleep, early morning awakening, tired awakening, loss of libido, restlessness, fatigue, constipation, dyspepsia, heart palpitations, headache, neck pain, back pain, limb pain, joint pain, abdominal pain, dizziness, nausea, heartburn, nervousness, tremors, burning and tingling sensations, morning stiffness, abdominal pain, abdominal distention, gurgling, diarrhea, and the symptoms associated with generalized anxiety disorder, comprising administering to said mammal in need of said treatment an amount a compound of Formula I, or a pharmaceutically acceptable salt or solvate thereof, wherein the amount of said compound of Formula I is effective in (1) antagonizing an NK-1 and/or NK-3 receptor, and/or (2) treating said condition or disorder.

In another aspect, the compound of formula I is used in an assay of NK-1 binding wherein said compound exhibits a Ki of about 5 nM or less, preferably 2 nM or less, more preferably about 0.1 nM or less.

In another practice, the compound of formula I is used in an assay of NK-3 binding wherein said compound exhibits a Ki of about 5 nM or less, preferably 2 nM or less, more preferably about 0.1 nM or less.

### DETAILED DESCRIPTION OF THE INVENTION

The present invention relates to a compound (that in various practices comprises piperidine, pyrrolidine, and diazepane derivatives) which is an antagonist of tachykinins, including substance P and other neurokinins (NK), such as NK-1, and is thus useful for the treatment of neurokinin-mediated conditions, among other things.

In a preferred embodiment, the compound of the invention has Formula I, above, including pharmaceutically acceptable salts thereof, e.g. acid addition salts, base addition salts, and prodrugs and solvates thereof. Without limitation, examples of pharmaceutically acceptable acid addition salts of the compounds of Formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, malate, di-p-toluoyl tartaric acid, lactic acid, acetic acid, trifluoroacetic acid, mandelic acid.

The compound of Formula I can have optical centers and thus occur in different enantiomeric configurations. The invention includes all enantiomers, diastereomers, and other stereoisomers and optical isomers of such compound of Formula I, as well as racemic and other mixtures thereof. For example, the compound of Formula I includes (R) and (S) enantiomers and cis and trans isomers. The present invention further includes all radiolabelled forms of the compound of Formula I. Preferred radiolabelled compounds are those wherein the radiolabels are selected from as ³H, ¹¹C, ¹⁴C, ¹⁸F, ¹²³I and ¹²⁵I. Such radiolabelled compounds are useful as research and diagnostic tools in metabolism pharmacokinetics studies and in binding assays in animals and man.

As appreciated by the artisan, the use of Formula I is a convenience and the invention is understood to envision and embrace each and every species thereunder as though individually identified and set forth herein. Thus the present invention severally contemplates each species separately and any and all combinations and permutations of species falling within Formula I.

In a first preferred practice of the compound of Formula I, L=0, n=0 or 1; m=0, s=0 or 1; R¹ and R² are each independently selected from H, CH₃, —(C₁-C₆)alkyl, —CH₂-aryl, —CH₂-heterocycloalkyl, or —CH₂-heteroaryl, wherein each of said —CH₂-aryl, —CH₂-heterocycloalkyl, or —CH₂-heteroaryl is optionally substituted with 1-3 moieties independently selected from X′, Y′ or Z′; R³ is H, R⁴ and R⁵ are each independently selected from H, CH₃, or —(C₁-C₆)alkyl; R⁶ is a bond, —CH₂—, —O—, or

—NR⁴—; R⁷ is (C₁-C₆)alkyl, OH, —N(R⁴)(R⁵), or —OR⁴; and X, Y, X′, Y′ and Z′ are each independently selected from H, (C₁-C₆)alkyl, CF₃, OH, —O—(C₁-C₆)alkyl, halogen, CN, —R⁶C(═O)R⁷ or —R⁶C(═O)NR⁴R⁵.

In a second preferred practice of the compound of Formula I, L=—NR⁴, s=0, n=0 or 1; m=1, R¹ and R² are each independently selected from H, CH₃, (C₂-C₆)alkyl, benzyl, —CH₂-heterocycloalkyl, or —CH₂-heteroaryl, wherein each of said benzyl, —CH₂-heterocycloalkyl, or —CH₂-heteroaryl is optionally substituted with 1-3 moieties independently selected from X′, Y′ and Z′; R³is H, R⁴ and R⁵ are each independently selected from H, CH₃, or —(C₁-C₆)alkyl; R⁶ is a bond, —CH₂—, —O—, or

—NR⁴—; R⁷ is (C₁-C₆)alkyl, OH, —N(R⁴)(R⁵), or —OR⁴; and X, Y, X′, Y′ and Z′ are each independently selected from H, (C₁-C₆)alkyl, CF₃, OH, —O—(C₁-C₆)alkyl, halogen, CN, —R⁶C(═O)R⁷ or —R⁶C(═O)NR⁴R⁵.

Specific compounds of Formula I that are NK-1 antagonists include:
4S-(3,5-bis-trifluoromethyl-benzloxy)-3R-2-tolyl-piperidine
4-(3,5-bis-trifluoromethyl-benzloxy)-3-phenyl-piperidine
4-(3,5-bis-trifluoromethyl-benzyloxy)-3-(4-fluoro-phenyl)-piperidine
4-(3,5-bis-trifluoromethyl-benzyloxy)-3-(2-methyl-4-fluoro-phenyl)-piperidine
4-(3,5-bis-trifluoromethyl-benzyloxy)-3-(3,4-difluoro-phenyl)-piperidine
4-(3,5-bis-trifluoromethyl-benzyloxy)-3-(2-pyridyl)-piperidine
4-(3,5-bis-trifluoromethyl-benzyloxy)-3-(3-chloro-phenyl)-piperidine
4-(4-bromo-benzyloxy)-3-(2-methyl-4-fluoro-phenyl)-piperidine
4-(3-bromo-benzyloxy)-3-(2-methyl-4-fluoro-phenyl)-piperidine
4-(2-bromo-benzyloxy)-3-(2-methyl-4-fluoro-phenyl)-piperidine
4-(2,6-dichloro-benzyloxy)-3-(2-methyl-4-fluoro-phenyl)-piperidine
4-(2,5-dichloro-benzyloxy)-3-(2-methyl-4-fluoro-phenyl)-piperidine
4-(3,5-dichloro-benzyloxy)-3-(2-methyl-4-fluoro-phenyl)-piperidine
4-(4-fluoro-benzyloxy)-3-(2-methyl-4-fluoro-phenyl)-piperidine
4-(2-fluoro-benzyloxy)-3-(2-methyl-4-fluoro-phenyl)-piperidine;
4-(3-fluoro-benzyloxy)-3-(2-methyl-4-fluoro-phenyl)-piperidine
4-(3,4-difluoro-benzyloxy)-3-(2-methyl-4-fluoro-phenyl)-piperidine
4-(3,4-difluoro-benzyloxy)-3-(2-methyl-4-fluoro-phenyl)-piperidine
4-(2,6-di-fluoro-benzyloxy)-3-(2-methyl-4-fluoro-phenyl)-piperidine
4-(3,6-di-fluoro-benzyloxy)-3-(2-methyl-4-fluoro-phenyl)-piperidine
4-(2,4,6-tri-fluoro-benzyloxy)-3-(2-methyl-4-fluoro-phenyl)-piperidine
4-(2,3,6-tri-fluoro-benzyloxy)-3-(2-methyl-4-fluoro-phenyl)-piperidine
4-(4-trifluoromethyl-benzyloxy)-3-(2-methyl-4-fluoro-phenyl)-piperidine
4-(3-trifluoromethyl-benzyloxy)-3-(2-methyl-4-fluoro-phenyl)-piperidine
4-(3,5-bis-trifluoromethyl-benzyloxy)-3-(2-methyl-4-fluoro-phenyl)-piperidine
4-(2,4-bis-trifluoromethyl-benzyloxy)-3-(2-methyl-4-fluoro-phenyl)-piperidine
4-(4-trifluoromethoxyl-benzyloxy)-3-(2-methyl-4-fluoro-phenyl)-piperidine
4-(2-methyl-3,4-Bisfluoromethyl-benzyloxy)-3-(2-methyl-4-fluoro-phenyl)-piperidine
4-(2-methyl-3,5-bis-fluoromethyl-benzyloxy)-3-(2-methyl-4-fluoro-phenyl)-piperidine
4-(2-ethyl-3,5-bis-fluoromethyl-benzyloxy)-3-(2-methyl-4-fluoro-phenyl)-piperidine
4-(2-methyl-benzyloxy)-3-(2-methyl-4-fluoro-phenyl)-piperidine
4-(2-chloro-5-methoxy-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid methylester
4-(3-methoxy-6-bromo-benzyloxy)-3-(2-methyl-4-fluoro-phenyl)-piperidine
4-(3-iodo-4-chloro-benzyloxy)-3-(2-methyl-4-fluoro-phenyl)-piperidine
4-(biphenyl-3-ylmethoxy)-3-(2-methyl-4-fluoro-phenyl)-piperidine
4-[2-(4-fluoro-benzyl)-benzyloxy]-3-(4-fluoro-2-methyl-phenyl)-piperidine
4-(3-methyl-benzyloxy)-3-(2-methyl-4-fluoro-phenyl)-piperidine
4-(2-trifluoromethyl-benzyloxy)-3-(2-methyl-4-fluoro-phenyl)-piperidine
4-(3-4-dimethyl-benzyloxy)-3-(2-methyl-4-fluoro-phenyl)-piperidine
4-(3-methyl-5-fluoro-benzyloxy)-3-(2-methyl-4-fluoro-phenyl)-piperidine
4-(2-methyl-3-fluoro-benzyloxy)-3-(2-methyl-4-fluoro-phenyl)-piperidine
4-(2,6-dibromo-benzyloxy)-3-(2-methyl-4-fluoro-phenyl)-piperidine
4-(3-chloro-6-methyl-benzyloxy)-3-(2-methyl-4-fluoro-phenyl)-piperidine
4-(2-iodo-benzyloxy)-3-(2-methyl-4-fluoro-phenyl)-piperidine
4-(2-isopropyl-benzyloxy)-3-(2-methyl-4-fluoro-phenyl)-piperidine
4-(3-fluoro-5-methyl-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid ethylester
4-benzyloxy-3-(2-methyl-4-fluoro-phenyl)-piperidine
3-[3-(2-methyl-4-fluoro-phenyl)]-piperidine-4-yloxymethyl-pyridine
3-[3-(2-methyl-4-fluoro-phenyl)]-piperidine-4-yloxymethyl-benzonitrile
1-[4S-(3,5-bis-trifluoromethyl-benzyloxy)-3R-2-tolyl-piperidine-1-yl]-2-(4-acetyl-piperazine-1-yl)-ethanone
1-[4S-(3,5-bis-trifluoromethyl-benzyloxy)-3R-2-tolyl-piperidine-1-yl]-2-pyrrolidin-1-yl-ethanone
1-[4-(2-ethyl-3,5-difluorobenzyloxy)-3-(4-fluoro-2-methyl-phenyl)-piperidine] 2-pyrrolidin-1-yl-ethanone
1-[4-(3,5-dichlorobenzyloxy)-3-(4-fluoro-2-methyl-phenyl)-piperidine] 2-pyrrolidin-1-yl-ethanone
1-[4-(3-methyl-5-fluorobenzyloxy)-3-(4-fluoro-2-methyl-phenyl)piperidine] 2-pyrrolidin-1-yl-ethanone
1-[4-(3-iodo-4-chlorobenzyloxy)-3-(4-fluoro-2-methyl-phenyl)piperidine] 2-pyrrolidin-1-yl-ethanone:

1-[4-(4,5-dichlorobenzyloxy)-3-(4-fluoro-2-methyl-phenyl)piperidine] 2-pyrrolidin-1-yl-ethanone
1-[4-(2-trifluoromethylbenzyloxy)-3-(4-fluoro-2-methyl-phenyl)piperidine] 2-pyrrolidin-1-yl-ethanone
1-[4-(5-iodobenzyloxy)-3-(4-fluoro-2-methyl-phenyl)piperidine] 2-pyrrolidin-1-yl-ethanone
1-[4-(biphenyl-2-ylmethoxy)-3-(4-fluoro-2-methyl-phenyl)piperidine] 2-pyrrolidin-1-yl-ethanone:
1-[4-(2-chloro-5-methoxybenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(4-iodobenzyloxy)-3-(4-fluoro-2-methyl-phenyl)piperidine] 2-pyrrolidin-1-yl-ethanone
1-[4-(5-bromobenzyloxy)-3-(4-fluoro-2-methyl-phenyl)piperidine] 2-pyrrolidin-1-yl-ethanone
1-[4-(2-methyl-5-fluorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2-iodobenzyloxy)-3-(4-fluoro-2-methyl-phenyl)piperidine] 2-pyrrolidin-1-yl-ethanone
1-[4-(2-difluoromethoxybenzyloxy)-3-(4-fluoro-2-methyl-phenyl)piperidine] 2-pyrrolidin-1-yl-ethanone:
1-[4-(2-methyl-5-chlorobenzyloxy)-3-(4-fluoro-2-methyl-phenyl)piperidine] 2-pyrrolidin-1-yl-ethanone
1-[4-(3-methylbenzyloxy)-3-(4-fluoro-2-methyl-phenyl)piperidine] 2-pyrrolidin-1-yl-ethanone
1-[4-(2-methyl-3,5-difluorobenzyloxy)-3-(4-fluoro-2-methyl-phenyl)piperidine] 2-pyrrolidin-1-yl-ethanone
1-[4-(3-methoxybenzyloxy)-3-(4-fluoro-2-methyl-phenyl)piperidine] 2-pyrrolidin-1-yl-ethanone
1-[4-(3-chlorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2,5-dichlorobenzyloxy)-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(4-bromobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2-methyl-3,4-difluorobenzyloxy)-3-(4-fluoro-2-methyl-phenyl)piperidine] 2-pyrrolidin-1-yl-ethanone
1-[4-(2-chlorobenzyloxy)-3-(4-fluoro-2-methyl-phenyl)piperidine] 2-pyrrolidin-1-yl-ethanone
1-[4-(2-methyl-3-fluorobenzyloxy)-3-(4-fluoro-2-methyl-phenyl)piperidine] 2-pyrrolidin-1-yl-ethanone
1-[4-(4-trifluoromethoxybenzyloxy)-3-(4-fluoro-2-methyl-phenyl)piperidine] 2-pyrrolidin-1-yl-ethanone
1-[4-(4-trifluoromethylbenzyloxy)-3-(4-fluoro-2-methyl-phenyl)piperidine] 2-pyrrolidin-1-yl-ethanone
1-[4-(2-fluorobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(2-cyanobenzyloxy)-3-(4-fluoro-2-methyl-phenyl)piperidine] 2-pyrrolidin-1-yl-ethanone
1-[4-(2,4,6-trifluorobenzyloxy)-3-(4-fluoro-2-methyl-phenyl)piperidine] 2-pyrrolidin-1-yl-ethanone
1-[4-(3-cyanobenzyloxy)-3-(4-fluoro-2-methyl-phenyl)phenyl] 2-pyrrolidin-1-yl-ethanone
1-[4-(5-methyl-6-fluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2,6-dichlorobenzyloxy)-3-(4-fluoro-2-methyl-phenyl)piperidine] 2-pyrrolidin-1-yl-ethanone
1-[4-(2,5,6-trifluorobenzyloxy)-3-(4-fluoro-2-methyl-phenyl)piperidine] 2-pyrrolidin-1-yl-ethanone
1-[4-(2,6-difluorobenzyloxy)-3-(4-fluoro-2-methyl-phenyl)piperidine] 2-pyrrolidin-1-yl-ethanone
1-[4-(benzyloxy)-3-(4-fluoro-2-methyl-phenyl)piperidine]-2-pyrrolidin-1-yl-ethanone
1-[(4-fluorophenoxy) benzyloxy-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(3-trifluoromethylbenzyloxy)-3-(4-fluoro-2-methyl-phenyl)piperidine] 2-pyrrolidin-1-yl-ethanone
1-[(4-benzoyl-benzyloxy)-3-(4-fluoro-2-methyl-phenyl)piperidine] 2-pyrrolidin-1-yl-ethanone
1-[4S-(2,4-bis-trifluoromethyl-benzyloxy)-3R-2-tolyl-piperidine-1-yl]-2-piperidine-1-yl-ethanone
1-[4S-(3,5-bis-trifluoromethyl-benzyloxy)-3R-2-tolyl-piperidine-1-yl]-2-morpholin-1-yl-ethanone
1-[4S-(3,5-bis-trifluoromethyl-benzyloxy)-3R-2-tolyl-piperidine-1-yl]-2-piperazine-1-yl-ethanone
1-[4S-(3,5-bis-trifluoromethyl-benzyloxy)-3R-2-tolyl-piperidine-1-yl]-2-(4-methyl-piperazine-1-yl)-ethanone
1-[4S-(3,5-bis-trifluoromethyl-benzyloxy)-3R-2-tolyl-piperidine-1-yl]-2-(4-ethyl-piperazine-1-yl)-ethanone
1-[4S-(3,5-bis-trifluoromethyl-benzyloxy)-3R-2-tolyl-piperidine-1-yl]-2-(4-benzyl-piperazine-1-yl)-ethanone
1-[4S-(3,5-bis-trifluoromethyl-benzyloxy)-3R-2-tolyl-piperidine-1-yl]-2-piperidine-1-carboxylic acid tert-butyl ester-1-yl-ethanone
1-[4S-(3,5-bis-trifluoromethyl-benzyloxy)-3R-2-tolyl-piperidine-1-yl]-2-piperidine-4-yl-ethanone
1-[4S-(3,5-bis-trifluoromethyl-benzyloxy)-3R-2-tolyl-piperidine-1-yl]-2-(1-Acetyl-piperidine-4-yl)-ethanone
1-[4S-(3,5-bis-trifluoromethyl-benzyloxy)-3R-2-tolyl-piperidine-1-yl]-2-(1-H-imidazole-4-yl)-ethanone
1-[4S-(3,5-bis-trifluoromethyl-benzyloxy)-3R-2-tolyl-piperidine-1-yl]-2-Pyridine-4-yl-ethanone
1-[4S-(3,5-bis-trifluoromethyl-benzyloxy)-3R-2-tolyl-piperidine-1-yl]-2-pyrrolidin-2-one-1-yl-ethanone
1-[4S-(3,5-bis-trifluoromethyl-benzyloxy)-3R-2-tolyl-piperidine-1-yl]-2-dimethylamino-1-yl-ethanone
[4S-(3,5-bis-trifluoromethyl-benzyloxy)-3R-2-tolyl-piperidine-1-yl]-(4-methyl-piperazine-1-yl)-methanone
[4S-(3,5-bis-trifluoromethyl-benzyloxy)-3R-2-tolyl-piperidine-1-yl]-piperidine-4-yl-methanone
[4S-(3,5-bis-trifluoromethyl-benzyloxy)-3R-2-tolyl-piperidine-1-yl]-piperidine-2-yl-methanone
[4S-(3,5-bis-trifluoromethyl-benzyloxy)-3R-2-tolyl-piperidine-1-yl]-thiazolidin-4-yl-methanone
[4-(3,5-bis-trifluoromethyl-benzyloxy)-3R-2-tolyl-piperidine-1-yl]-(2-hydroxy-pyridine-3-yl)-methanone
[4-(3,5-bis-trifluoromethyl-benzyloxy)-3R-2-tolyl-piperidine-1-yl]-(3-hydroxy-pyridine-2-yl)-methanone
1-{2-[4S-(3,5-bis-trifluoromethyl-benzyloxy)-3R-2-tolyl-piperidine-1-carbonyl]-4R-hydroxy-pyrrolidine-1-yl)}-ethanone
[4S-(3,5-bis-trifluoromethyl-benzyloxy)-3R-2-tolyl-piperidine-1-yl]-pyridine-4-yl-methanone
1-{2-[4S-(3,5-bis-trifluoromethyl-benzyloxy)-3R-2-tolyl-piperidine-1-carbonyl]-pyrrolidin-1-yl)}-ethanone
[4S-(3,5-bis-trifluoromethyl-benzyloxy)-3R-2-tolyl-piperidine-1-yl]-pyrrolidin-1-yl-methanone
[4S-(3,5-bis-trifluoromethyl-benzyloxy)-3R-2-tolyl-piperidine-1-yl]-morpholin-4-yl-methanone
[4S-(3,5-bis-trifluoromethyl-benzyloxy)-3R-2-tolyl-piperidine-1-yl]-2-dimethylamino-ethanone
N-{2-[4S-(3,5-bis-trifluoromethyl-benzyloxy)-3R-2-tolyl-piperidine-1-yl]-2oxo-ethyl}-acetamide
2-amino-1-[4S-(3,5-bis-trifluoromethyl-benzyloxy)-3R-2-tolyl-piperidine-1-yl]-ethanone
2-[4S-(3,5-bis-trifluoromethyl-benzyloxy)-3R-2-tolyl-piperidine-1-yl]-N-N-dimethyl-acetamide
4-[4S-(3,5-bis-trifluoromethyl-benzyloxy)-3R-2-tolyl-piperidine-1-carbonyl]-piperidine-2,6-dione
[4S-(3,5-bis-trifluoromethyl-benzyloxy)-3R-2-tolyl-piperidine-1-yl]-pyrrolidin-2-yl-methanone
[4S-(3,5-bis-trifluoromethyl-benzyloxy)-3R-2-tolyl-piperidine-1-yl]-piperazine-2-yl-methanone
5-[4S-(3,5-bis-trifluoromethyl-benzyloxy)-3R-2-tolyl-piperidine-1-methyl]-2-4-dihydro-[1,2,4] triazol-3-one
5-[4S-(3,5-bis-trifluoromethyl-benzyloxy)-3R-(3,4-difluoro-phenyl)-2-piperidine-1-methyl]-2-4-dihydro-[1,2,4] triazol-3-one
5-[4S-(3,5-bis-trifluoromethyl-benzyloxy)-3R-phenyl-2-piperidine-1-methyl]-2-4- dihydro-[1,2,4] triazol-3-one
5-[4S-(3,5-dimethyl-benzyloxy)-3R-phenyl-2-piperidine-1-methyl]-2-4-dihydro-[1,2,4] triazol-3-one
5-[4-(3,5-bis-trifluoromethyl-benzyloxy)-3-(4-fluoro-phenyl)-2-piperidine-1-methyl]-2-4-dihydro-[1,2,4] triazol-3-one
5-[4-(3,5-bis-fluoromethyl-benzyloxy)-3-phenyl)-2-piperidine-1-methyl]-2-4-dihydro-[1,2,4] triazol-3-one
4-(3,5-bis-trifluoromethyl-benzloxy)-1-methyl-3-tolyl-piperidine
1-[4-(3,5-bis-trifluoromethyl-benzloxy)-3-(4-fluoro-2-methyl-phenyl)-piperidine-1-yl]-ethanone
1-[4S-(3,5-bis-trifluoromethyl-benzyloxy)-3R-2-tolyl-piperidine-1-yl]-ethanone
{5-[4-(3,5-bis-trifluoromethyl-benzloxy)-3-phenyl-piperidine-1-yl]-2H[1,2,3] triazol-4-methyl}dimethyl-amine
{5-[4-(3,5-bis-trifluoromethyl-benzloxy)-3-(4-fluoro-phenyl)-piperidine-1-yl]-2H[1,2,3] triazol-4-methyl} dimethyl-amine
{5-[4-(3,5-dimethyl-benzyloxy)-3-phenyl-piperidine-1-yl]-2H[1,2,3] triazol-4-methyl}dimethyl-amine
   or pharmaceutically acceptable salts or solvates thereof.

Preferred NK-1 antagonists from the above list include:
4S-(3,5-bis-trifluoromethyl-benzloxy)-3R-2-tolyl-piperidine
4-(3,5-bis-trifluoromethyl-benzloxy)-3-phenyl-piperidine
4-(3,5-bis-trifluoromethyl-benzyloxy)-3-(4-fluoro-phenyl)-piperidine
4-(3,5-bis-trifluoromethyl-benzyloxy)-3-(2-methyl-4-fluoro-phenyl)-piperidine
4-(3,5-bis-trifluoromethyl-benzyloxy)-3-(3,4-difluoro-phenyl)-piperidine
4-(3,5-bis-trifluoromethyl-benzyloxy)-3-(2-pyridyl)-piperidine
4-(3,5-bis-trifluoromethyl-benzyloxy)-3-(3-chloro-phenyl)-piperidine
1-[4S-(3,5-bis-trifluoromethyl-benzyloxy)-3R-2-tolyl-piperidine-1-yl]-2-morpholin-1-yl-ethanone
1-[4S-(3,5-bis-trifluoromethyl-benzyloxy)-3R-2-tolyl-piperidine-1-yl]-2-piperazine-1-yl-ethanone
1-[4S-(3,5-bis-trifluoromethyl-benzyloxy)-3R-2-tolyl-piperidine-1-yl]-2-(4-methyl-piperazine-1-yl)-ethanone
1-[4S-(3,5-bis-trifluoromethyl-benzyloxy)-3R-2-tolyl-piperidine-1-yl]-2-(4-ethyl-piperazine-1-yl)-ethanone
1-[4S-(3,5-bis-trifluoromethyl-benzyloxy)-3R-2-tolyl-piperidine-1-yl]-2-(4-benzyl-piperazine-1-yl)-ethanone
1-[4S-(3,5-bis-trifluoromethyl-benzyloxy)-3R-2-tolyl-piperidine-1-yl]-2-piperidine-1-carboxylic acid tert-butyl ester-1-yl-ethanone
1-[4S-(3,5-bis-trifluoromethyl-benzyloxy)-3R-2-tolyl-piperidine-1-yl]-2-piperidine-4-yl-ethanone
1-[4S-(3,5-bis-trifluoromethyl-benzyloxy)-3R-2-tolyl-piperidine-1-yl]-2-(1-acetyl-piperidine-4-yl)-ethanone
1-[4S-(3,5-bis-trifluoromethyl-benzyloxy)-3R-2-tolyl-piperidine-1-yl]-2-(1-H-imidazole-4-yl)-ethanone
1-[4S-(3,5-bis-trifluoromethyl-benzyloxy)-3R-2-tolyl-piperidine-1-yl]-2-pyridine-4-yl-ethanone
1-[4S-(3,5-bis-trifluoromethyl-benzyloxy)-3R-2-tolyl-piperidine-1-yl]-2-pyrrolidin-2-one-1-yl-ethanone
1-[4S-(3,5-bis-trifluoromethyl-benzyloxy)-3R-2-tolyl-piperidine-1-yl]-2-dimethylamino-1-yl-ethanone
[4S-(3,5-bis-trifluoromethyl-benzyloxy)-3R-2-tolyl-piperidine-1-yl]-(4-methyl-piperazine-1-yl)-methanone
[4S-(3,5-bis-trifluoromethyl-benzyloxy)-3R-2-tolyl-piperidine-1-yl]-piperidine-4-yl-methanone
[4S-(3,5-bis-trifluoromethyl-benzyloxy)-3R-2-tolyl-piperidine-1-yl]-piperidine-2-yl-methanone
[4S-(3,5-bis-trifluoromethyl-benzyloxy)-3R-2-tolyl-piperidine-1-yl]-pyrrolidin-1-yl-methanone
[4S-(3,5-bis-trifluoromethyl-benzyloxy)-3R-2-tolyl-piperidine-1-yl]-morpholin-4-yl-methanone
[4S-(3,5-bis-trifluoromethyl-benzyloxy)-3R-2-tolyl-piperidine-1-yl]-2-dimethylamino-ethanone
2-[4S-(3,5-bis-trifluoromethyl-benzyloxy)-3R-2-tolyl-piperidine-1-yl]-N-N-dimethyl-acetamide
5-[4S-(3,5-bis-trifluoromethyl-benzyloxy)-3R-2-tolyl-piperidine-1-methyl]-2-4-dihydro-[1,2,4] triazol-3-one
5-[4S-(3,5-bis-trifluoromethyl-benzyloxy)-3R-(3,4-difluoro-phenyl)-2-piperidine-1-methyl]-2-4-dihydro-[1,2,4] triazol-3-one
5-[4S-(3,5-bis-trifluoromethyl-benzyloxy)-3R-phenyl-2-piperidine-1-methyl]-2-4-dihydro-[1,2,4] triazol-3-one
5-[4-(3,5-bis-trifluoromethyl-benzyloxy)-3-(4-fluoro-phenyl)-2-piperidine-1-methyl]-2-4-dihydro-[1,2,4] triazol-3-one
1-[4S-(3,5-bis-trifluoromethyl-benzyloxy)-3R-2-tolyl-piperidine-1-yl]-ethanone
   or pharmaceutically acceptable salts or solvates thereof.

Specific examples of compounds of formula I that are NK-3 antagonists include:
3-(4-fluoro-2-methyl-phenyl)-4-(2-phenyl-butyrylamino)-piperidine
4-oxo-2,4-diphenyl-(3-phenyl-piperidin-4-yl)-butyramide
2-(4-nitro-phenyl) -(3-phenyl-piperidin-4-yl)-propionamide
2-phenyl-(3-phenyl-piperidin-4-yl)-propionamide
1,2,3,4-tetrahydro-naphthalene-1-carboxylic acid (3-phenyl-piperidin-4-yl)-amide:
3-methyl-2-phenyl-(3-phenyl-piperidin-4-yl)-butyramide
2-phenyl-(3-phenyl-piperidin-4-yl)-butyramide
2-(3-benzoyl-phenyl)-(3-phenyl-piperidin-4-yl)-propionamide
(3-phenyl-piperidin-4-yl)-2-tolyl-acetamide
(3-phenyl-piperidin-4-yl)-2-[4-(thiophene-2-carbonyl)-phenyl]-propionamide
6-fluoro-2-oxo-1,2,3,4-tetrahydro-quinoline-4-carboxylic acid (3-phenyl-piperidin-4-yl)-amide
3-furan-2-yl-2-phenyl-(3-phenyl-piperidin-4-yl)-propionamide
6-chloro-8-methyl-chroman-4-carboxylic acid (3-phenyl-piperidin-4-yl)-amide
5-cyclohexyl-indan-1-carboxylic acid (3-phenyl-piperidin-4-yl)-amide
2-(3,4-dimethoxy-phenyl)-hexanoic acid (3-phenyl-piperidin-4-yl)-amide
6-methyl-indan-1-carboxylic acid (3-phenyl-piperidin-4-yl)-amide
2-[3-chloro-4-(2,5-dihydro-pyrrol-1-yl)-phenyl-(3-phenyl-piperidin-4-yl)-propionamide
6-methoxy-3-oxo-indan-1-carboxylic acid (3-phenyl-piperidin-4-yl)-amide
6,7-dichloro-chroman-4-carboxylic acid (3-phenyl-piperidin-4-yl)-amide
2-{4-[2-(4-methoxy-phenyl)-vinyl]-phenyl-(3-phenyl-piperidin-4-yl)-propionamide:
2-phenyl-(3-phenyl-piperidin-4-yl)-propionamide
2-(4-chloro-phenyl-3-phenyl-piperidin-4-yl)-propionamide
2-phenyl-hexanoic acid (3-phenyl-piperidin-4-yl)-amide
thiochroman-4-carboxylic acid (3-phenyl-piperidin-4-yl)-amide
5-methoxy-1,2,3,4-tetrahydro-naphthalene-1-carboxylic acid (3-phenyl-piperidin-4-yl)-amide
1-oxo-3-phenyl-1,2,3,4-tetrahydro-isoquinoline-4-carboxylic acid (3-phenyl-piperidin-4-yl)-amide
6-methoxy-2-methyl-1,2,3,4-tetrahydro-isoquinoline-4-carboxylic acid (3-phenyl-piperidin-4-yl)-amide
2-(4-hydroxy-phenyl)-3-phenyl-3-phenyl-piperidin-4-yl)-propionamide:
2-(2-fluoro-biphenyl-4-yl-3-phenyl-piperidin-4-yl)-propionamide
chroman-2-carboxylic acid (3-phenyl-piperidin-4-yl)-amide
2,4-diphenyl-3-phenyl-piperidin-4-yl)-butyramide
6-chloro-thiochroman-4-carboxylic acid (3-phenyl-piperidin-4-yl)-amide
7-methoxy-chroman-4-carboxylic acid (3-phenyl-piperidin-4-yl)-amide
2-[4-(2-hydroxy-2-methyl-propyl)-phenyl]-(3-phenyl-piperidin-4-yl)-propionamide:
2-phenyl-(3-phenyl-piperidin-4-yl)-butyramide
2-(4-hydroxy-phenyl-3-phenyl-piperidin-4-yl)-propionamide
2-phenyl-3-phenyl-piperidin-4-yl)-acetamide
2,3-diphenyl-(3-phenyl-piperidin-4-yl)-propionamide
2-(3-phenoxy-phenyl-3-phenyl-piperidin-4-yl)-propionamide
2-(4-isobutyl-phenyl-3-phenyl-piperidin-4-yl)-propionamide
2-phenyl-3-phenyl-piperidin-4-yl)-propionamide
indan-1-carboxylic acid (3-phenyl-piperidin-4-yl)-amide
2-phenoxy -(3-phenyl-piperidin-4-yl)-propionamide
3-(4-methoxy-phenyl)-2-phenyl-(3-phenyl-piperidin-4-yl)-propionamide
2-cyclopentyl-2-phenyl-(3-phenyl-piperidin-4-yl)-acetamide
1,2,3,4-tetrahydro-isoquinoline-4-carboxylic acid (3-phenyl-piperidin-4-yl)-amide
2-phenyl-3-(5-phenyl-furan-2-yl-3-phenyl-piperidin-4-yl)-propionamide
3-(4-hydroxy-phenyl)-2-phenyl-3-phenyl-piperidin-4-yl)-propionamide
6,7-dichloro-thiochroman-4-carboxylic acid (3-phenyl-piperidin-4-yl)-amide
6-fluoro-3-hydroxy-indan-1-carboxylic acid (3-phenyl-piperidin-4-yl)-amide
4,5,6,7-tetramethyl-3-oxo-indan-1-carboxylic acid (3-phenyl-piperidin-4-yl)-amide
2-(2,5-dimethyl-phenyl)-6-phenyl-hexanoic acid (3-phenyl-piperidin-4-yl)-amide
3-methyl-2-phenyl-pentanoic acid (3-phenyl-piperidin-4-yl)-amide
(3-phenyl-piperidin-4-yl)-2-tolyl-butyramide
6-fluoro-chroman-4-carboxylic acid (3-phenyl-piperidin-4-yl)-amide
7-methoxy-1,2,3,4-tetrahydro-naphthalene-1-carboxylic acid (3-phenyl-piperidin-4-yl)-amide
3-oxo-indan-1-carboxylic acid (3-phenyl-piperidin-4-yl)-amide
2-biphenyl-4-yl-pent-4-enoic acid (3-phenyl-piperidin-4-yl)-amide
2-naphthalen-1-yl-heptanoic acid (3-phenyl-piperidin-4-yl)-amide
2-(6-methoxy-naphthalen-2-yl-3-phenyl-piperidin-4-yl)-propionamide
2-(4-chloro-phenyl)-3-methyl-3-phenyl-piperidin-4-yl)-butyramide
5-methyl-2-tolyl-hexanoic acid (3-phenyl-piperidin-4-yl)-amide
6-methoxy-1,2,3,4-tetrahydro-naphthalene-1-carboxylic acid (3-phenyl-piperidin-4-yl)-amide
6-methoxy-chroman-4-carboxylic acid (3-phenyl-piperidin-4-yl)-amide
6-fluoro-3-oxo-indan-1-carboxylic acid (3-phenyl-piperidin-4-yl)-amide
3-hydroxy-2-phenyl-(3-phenyl-piperidin-4-yl)-propionamide
4-(4-methoxy-phenyl)-4-oxo-2-phenyl-(3-phenyl-piperidin-4-yl)-butyramide
6-chloro-9-methyl-2,3,4,9-tetrahydro-1-carbazole-4-carboxylic acid (3-phenyl-piperidin-4-yl)-amide
2-(4-isobutyl-phenyl-3-phenyl-piperidin-4-yl)-propionamide
2-phenoxy-3-phenyl-piperidin-4-yl)-butyramide
2-biphenyl-4-yl-hex-4-enoic acid (3-phenyl-piperidin-4-yl)-amide
2-cyclohex-2-enyl-2-phenyl-3-phenyl-piperidin-4-yl)-acetamide
6,7-dimethyl-chroman-4-carboxylic acid (3-phenyl-piperidin-4-yl)-amide
2-[2-(4-chloro-phenyl)-benzooxazol-5-yl-3-phenyl-piperidin-4-yl]-propionamide
3-(4-hydroxy-3,5-diiodo-phenyl)-2-phenyl-3-phenyl-piperidin-4-yl)-propionamide
2-(4-methoxy-phenyl)-3-(5-phenyl-furan-2-yl)-(3-phenyl-piperidin-4-yl)-propionamide
6-chloro-chroman-4-carboxylic acid (3-phenyl-piperidin-4-yl)-amide
4,5-dimethoxy-indan-1-carboxylic acid (3-phenyl-piperidin-4-yl)-amide
6,7-dichloro-2-methyl-chroman-4-carboxylic acid (3-phenyl-piperidin-4-yl)-amide
2-(6-hydroxy-naphthalen-2-yl-3-phenyl-piperidin-4-yl)-propionamide
   or pharmaceutically acceptable salts or solvates of thereof.

Preferred NK-3 compounds from the above list include:
3-(4-fluoro-2-methyl-phenyl)-4-(2-phenyl-butyrylamino)-piperidine
2-phenyl-(3-phenyl-piperidin-4-yl)-propionamide
2,4-diphenyl-3-phenyl-piperidin-4-yl)-butyramide
2-phenyl-(3-phenyl-piperidin-4-yl)-butyramide
2-phenyl-3-phenyl-piperidin-4-yl)-acetamide
2,3-diphenyl-(3-phenyl-piperidin-4-yl)-propionamide
2-(phenyl-3-phenyl-piperidin-4-yl)-propionamide
(3-phenyl-piperidin-4-yl)-2-tolyl-butyramide
   or pharmaceutically acceptable salts or solvates thereof.

The specific NK-1 and NK-3 antagonists listed above may act as both NK-1 and NK-3 antagonists.

The present invention is also directed to a pharmaceutical composition comprising the compound of the invention; and a pharmaceutically acceptable carrier.

Unless otherwise indicated, the following terms and related variations of same as used herein representatively have the meanings ascribed:

“Halogen” and “halo” and the like includes fluoro, chloro, bromo and iodo.

“Alkyl” including as appears in any terms such as “alkoxy” and “alkyoxycarbonyl,” or in any substituents such as —O—(C₁-C₆)alkyl, —O—(C₁-C₆)alkyl, or —(C₁-C₆)alkyl-C(O)—R⁶ includes saturated monovalent hydrocarbon radicals having straight or branched moieties. The alkyl moieties can include one or more points of unsaturation wherein the alkyl moieties can have carbon-carbon double bond or triple bonds; e.g. ethenyl, ethynyl, propenyl and propynyl.

Examples of alkyl groups include, but are not limited to, methyl, ethyl, n-propyl, isopropyl, and t-butyl.

“Alkoxycarbonyl” is —C(═O)-13 OR^{A} wherein R^{A} is (C₁-C₆)alkyl as defined herein.

“Ring system substituent” means a substituent attached to an aromatic or non-aromatic ring system which, for example, replaces an available hydrogen on the ring system. Ring system substituents can be the same or different, each being independently selected from the group consisting of alkyl, cycloalkyl, aryl, —O-aryl, heteroaryl, aralkyl, alkylaryl, heteroaralkyl, alkylheteroaryl, hydroxy, hydroxyalkyl, (C₁-C₆)alkoxy, aryloxy, aralkoxy, acyl, aroyl, halo, nitro, cyano, carboxy, and heterocyclyl.

“Cycloalkyl” includes non-aromatic saturated cyclic alkyl moieties wherein alkyl is as defined above. The cycloalkyl can be optionally substituted with one or more “ring system substituents” which can be the same or different, and are as defined above. Examples of cycloalkyl include, but are not limited to, cyclopropyl, cyclobutyl, cyclopentyl, cyclohexyl, and cycloheptyl; and bicycloalkyl and tricycloalkyl groups that are non-aromatic saturated carbocyclic groups consisting of two or three rings respectively, wherein said rings share at least one carbon atom. For purposes of the present invention, and unless otherwise indicated, bicycloalkyl groups include spiro groups and fused ring groups. Examples of bicycloalkyl groups include, but are not limited to, bicyclo-[3.1.0]-hexyl, bicyclo-2.2.1]-hept-1-yl, norbornyl, spiro[4.5]decyl, spiro[4.4]nonyl, spiro[4.3]octyl, and spiro[4.2]heptyl. An example of a tricycloalkyl group is adamantanyl. Cycloalkyl groups also include groups that are substituted with one or more oxo moieties. Examples of such groups with oxo moieties are oxocyclopentyl and oxocyclohexyl.

“Aryl” refers to monocyclic and multicyclic groups which includes an organic radical derived from an aromatic hydrocarbon by removal of one hydrogen, such as phenyl, naphthyl, tetrahydonaphthyhl, indenyl, indanyl, and fluorenyl; and fused ring groups wherein at least one ring is aromatic. The aryl groups can be optionally substituted with one or more “ring system substituents” which can be the same or different, and are as defined above. The aryl groups of this invention can also include ring systems substituted with one or more oxo moieties.

“Oxo” is =O.

“Heterocyclic” refers to non-aromatic cyclic groups containing one or more heteroatoms, preferably from one to four heteroatoms, each selected from O, S and N. The heterocyclic can be optionally substituted with one or more “ring system substituents” which can be the same or different, and are as defined above. Heterocyclic groups also include ring systems substituted with one or more oxo moieties. Examples of heterocyclic groups are aziridinyl, azetidinyl, pyrrolidinyl, dihydropyrolyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

“Heteroaryl” refers to aromatic groups containing one or more heteroatoms (O, S, or N), preferably from one to four heteroatoms. The heteroaryl can be optionally substituted with one or more “ring system substituents” which can be the same or different, and are as defined above. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, 1,2,3,4-tetrahydroquinolyl, tetrazolyl, furyl, furanyl, thienyl, isoxazolyl, thiazolyl, chromanyl, thiochromanyl, thiophenyl, oxazolyl, isothiazolyl, pyrrolyl, indolyl, benzimidazolyl, benzofuranyl, cinnolinyl, indazolyl, indolizinyl, phthalazinyl, triazinyl, 1,2,4-triazinyl, 1,3,5-triazinyl, isoindolyl, 1-oxoisoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

“Heterobicyclic” refers to non-aromatic two-ringed cyclic groups, including bridged ring systems, wherein at least one of the rings contains a heteroatom of O, S or N, including without limitation azabicyclics such as 3-azabicyclo[3.1.0]hexanyl and 3-azabicyclo[4.1.0]heptanyl. The heterobicyclic can be optionally substituted with one or more “ring system substituents” which can be the same or different, and are as defined above.

The foregoing groups, as derived from the compounds listed above, can be C-attached or N-attached where such is possible. For instance, a group derived from pyrrole can be pyrrol-1-yl (N-attached) or pyrrol-3-yl (C-attached). The terms referring to the groups also encompass all possible tautomers.

“Solvates” of the compounds of the invention are also contemplated herein. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂.

“Treatment” and “treating” refers to reversing, alleviating, inhibiting the progress of, or preventing the disorder or condition to which such term applies, or one or more symptoms of such condition or disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder. The term “treatment”, as used herein, refers to the act of treating, as “treating” is defined immediately above.

“Mammal” refers to any member of the class “Mammalia”, including, but not limited to, humans, dogs, and cats.

### NK-Mediated Conditions

The present invention also relates to a method of treating one or more disorders or conditions such as sleep disorders (e.g., sleep apnea, insomnia, somnambulism, sleep deprivation, REM sleep disorders, hypersomnia, parasomnias, sleep-wake cycle disorders, narcolepsy, sleep disorders associated with shift work or irregular work schedules, and other sleep disorders); pervasive development disorder; rheumatoid arthritis; osteoarthritis; fibromyalgia; human immunodeficiency virus (HIV) infections; dissociative disorders such as body dysmorphic disorders; eating disorder such as anorexia and bulimia; ulcerative colitis; Crohn's disease; irritable bowel syndrome; functional abdominal pain; chronic fatigue syndrome; sudden infant death syndrome (SIDS); overactive bladder; chronic cystitis; chemotherapy induced cystitis; cough, angiotensin converting enzyme (ACE) induced cough; itch; hiccups; premenstrual syndrome: premenstrual dysphoric disorder; schizophrenia; schizoaffective disorder; delusional disorder; substance-induced psychotic disorder; brief psychotic disorder; shared psychotic disorder; psychotic disorder due to a general medical condition; schizophreniform disorder; amenorrheic disorders such as desmenorrhea; obesity; epilepsy: movement disorders such as primary movement disorders, spasticities, Scott's syndrome, Tourette's syndrome, palsys (e.g., Bell's palsy, cerebral palsy, birth palsy, brachial palsy, wasting palsy, ischemic palsy, progressive bulbar palsy and other palsys), amyolateral sclerosis (ALS), akinetic-rigid disorders, akinesias, dyskinesias (e.g., familial paroxysmal dyskinesia, tardive dyskinesia, tremor, chorea, myoclonus, tics and other dyskinesias) restless leg syndrome and movement disorders associated with Parkinson's disease or Huntington's disease; mastalgia syndromes; motion sickness; immune dysfunctions (e.g., stress induced immune dysfunctions such as idiopathic immune dysfunctions, post infection immune dysfunctions, post lumpectomy immune dysfunctions, porcine stress syndrome, bovine shipping fever, equine paroxysmal fibrillation, confinement dysfunction in chicken, sheering stress in sheep, and human-animal interaction stress in dogs); generalized anxiety disorder; panic disorder; phobias, including social phobia, agoraphobia, and specific phobias; obsessive-compulsive disorder; post-traumatic stress disorder; depression including major depressive disorder, single episode depression, recurrent depression, child abuse induced depression, postpartum depression and dysthymia; cyclothymia; bipolar disorder; neurocardiac disorders such as neurocardiac syncope, neurogenic syncope, hypersensitive Carotid sinus, neurovascular syndrome and arrythmias including arrythmias secondary to gastrointestinal disturbances; addiction disorders involving addictions to behaviors (e.g., addictions to gambling and other addictive behaviors); HIV-1 associated dementia; HIV encephalopathy; AIDS dementia complex (ADC); HIV related neuralgias; AIDS related neuralgias; epilepsy; and attention deficit hyperactivity disorder in a mammal, including a human, comprising administering to said mammal an amount of a compound of Formula I, as defined above, or a pharmaceutically acceptable salt thereof, that is effective in antagonizing the effect of NK-1 and/or NK-3 at its receptor sites.

Other more specific methods of this invention include any of the above methods wherein the disorder or condition that is being treated is selected from movement disorders such as primary movement disorders, spasticities, Scott's syndrome, Tourette's syndrome, palsys (e.g., Bell's palsy, cerebral palsy, birth palsy, brachial palsy, wasting palsy, ischemic palsy, progressive bulbar palsy and other palsys), amyolateral sclerosis (ALS), akinetic-rigid disorders, akinesias, dyskinesias (e.g., familial paroxysmal dyskinesia, tardive dyskinesia, tremor, chorea, myoclonus, tics and other dyskinesias) restless leg syndrome and movement disorders associated with Parkinson's disease or Huntington's disease.

Other more specific methods of this invention include the above methods wherein the disorder or condition that is being treated is major depressive disorder.

Other more specific methods of this invention include the above methods wherein the disorder or condition that is being treated is major depressive disorder, and wherein the mammal being treated is a human who has not exhibited an adequate treatment response following treatment for the same disorder or condition with a selective serotonin reuptake inhibitor (SSRI). The phrase “adequate treatment response” to an SSRI, as used herein, means that the SSRI with which the human patient was treated in accordance with a treatment protocol accepted by those of skill in the art of treating the disorder or condition for which such patient was being treated did not result in a degree of amelioration of the symptoms of such disorder or condition that would cause such persons of skill in the art to consider such treatment successful.

Other more specific methods of this invention include the above methods wherein the disorder or condition that is being treated is somatic major depressive disorder.

Other more specific methods of this invention include the above methods wherein the disorder or condition that is being treated is somatic major depressive disorder, and wherein the mammal being treated is a human who has not exhibited an adequate treatment response following treatment for the same disorder or condition with a selective serotonin reuptake inhibitor (SSRI). The phrase “adequate treatment response” to an SSRI, as used herein, means that the SSRI with which the human patient was treated in accordance with a treatment protocol accepted by those of skill in the art of treating the disorder or condition for which such patient was being treated did not result in a degree of amelioration of the symptoms of such disorder or condition that would cause such persons of skill in the art to consider such treatment successful.

Other more specific methods of this invention include the above methods wherein the disorder or condition that is being treated is irritable bowel syndrome.

Other more specific methods of this invention include the above methods wherein the disorder or condition that is being treated is an HIV infection.

Other more specific methods of this invention include the above methods wherein the disorder or condition that is being treated is selected from HIV-1 associated dementia, AIDS dementia complex (ADC), HIV encephalopathy, and HIV related neuralgias.

Other more specific methods of this invention include the above methods wherein the disorder or condition that is being is immune dysfunctions (e.g., stress induced immune dysfunctions such as idiopathic immune dysfunctions, post infection immune dysfunctions, post lumpectomy immune dysfunctions, porcine stress syndrome, bovine shipping fever, equine paroxysmal fibrillation, confinement dysfunction in chicken, sheering stress in sheep, or human-animal interaction stress in dogs).

Other more specific methods of this invention include the above methods wherein the disorder or condition that is being treated is neurocardiac disorders such as neurocardiac syncope, neurogenic syncope, hypersensitive Carotid sinus, neurovascular syndrome or arrythmias including arrythmias secondary to gastrointestinal disturbances.

Other more specific methods of this invention include the above methods wherein the disorder or condition that is being treated is major depression, single episode depression, recurrent depression, child abuse induced depression, postpartum depression, dysthymia, cyclothymia or bipolar disorder.

Other more specific methods of this invention include the above methods wherein the disorder or condition that is being treated is major depression, single episode depression, recurrent depression, child abuse induced depression, postpartum depression, dysthymia, cyclothymia or bipolar disorder, wherein the mammal being treated is a human who has; not exhibited an adequate treatment response following treatment for the same disorder or condition with a selective serotonin reuptake inhibitor (SSRI). The phrase “adequate treatment response” to an SSRI, as used herein, means that the SSRI with which the human patient was treated in accordance with a treatment protocol accepted by those of skill in the art of treating the disorder or condition for which such patient was being treated did not result in a degree of amelioration of the symptoms of such disorder or condition that would cause such persons of skill in the art to consider such treatment successful.

Other more specific methods of this invention include the above methods wherein the disorder or condition that is being treated is body dysmorphic disorders and eating disorders such as anorexia and bulimia.

Other more specific methods of this invention include the above methods wherein the disorder or condition that is being treated is schizophrenia, schizoaffective disorder, delusional disorder, substance-induced psychotic disorder, brief psychotic disorder, shared psychotic disorder, psychotic disorder due to a general medical condition, or schizophreniform disorder.

Other more specific methods of this invention include the above methods wherein the disorder or condition that is being treated is premenstrual syndrome, premenstrual dysphoric disorder, and amenorrheic disorders such as desmenorrhea.

Other more specific methods of this invention include the above methods wherein the disorder or condition that is being treated is premenstrual syndrome, premenstrual dysphoric disorder, or amenorrheic disorders such as desmenorrhea, wherein the mammal being treated is a human who has not exhibited an adequate treatment response following treatment for the same disorder or condition with a selective serotonin reuptake inhibitor (SSRI). The phrase “adequate treatment response” to an SSRI, as used herein, means that the SSRI with which the human patient was treated in accordance with a treatment protocol accepted by those of skill in the art of treating the disorder or condition for which such patient was being treated did not result in a degree of amelioration of the symptoms of such disorder or condition that would cause such persons of skill in the art to consider such treatment successful.

Other more specific methods of this invention include the above methods wherein the disorder or condition that is being treated is Crohn's disease, irritable bowel syndrome or functional abdominal pain.

Other more specific methods of this invention include the above methods wherein the disorder or condition that is being treated is selected from autism, pervasive development disorder, or attention deficit hyperactivity disorder.

Other more specific method of this invention include the above methods wherein the disorder or condition that is being treated is selected from chronic fatigue syndrome, sudden infant death syndrome (SIDS), obesity, or epilepsy.

Other more specific methods of this invention include the above methods wherein the disorder or condition that is being treated is generalized anxiety disorder, panic disorder, obsessive-compulsive disorder, post-traumatic stress disorder, or phobias, including social phobia, agoraphobia, and specific phobias.

Other more specific methods of this invention include the above methods wherein the disorder or condition that is being treated is generalized anxiety disorder, panic disorder, obsessive-compulsive disorder, post-traumatic stress disorder, and phobias, including social phobia, agoraphobia, or specific phobias, wherein the mammal being treated is a human who has not exhibited an adequate treatment response following treatment for the same disorder or condition with a selective serotonin reuptake inhibitor (SSRI). The phrase “adequate treatment response” to an SSRI, as used herein, means that the SSRI with which the human patient was treated in accordance with a treatment protocol accepted by those of skill in the art of treating the disorder or condition for which such patient was being treated did not result in a degree of amelioration of the symptoms of such disorder or condition that would cause such persons of skill in the art to consider such treatment successful.

Other more specific methods of this invention include the above methods wherein the disorder or condition that is being treated is cough, angiotensin converting enzyme (ACE) induced cough, itch, or hiccups.

Other more specific methods of this invention include the above methods wherein the disorder or condition that is being treated is overactive bladder; chronic cystitis or chemotherapy induced cystitis.

Other more specific methods of this invention include the above methods wherein the disorder or condition that is being treated is attention deficit hyperactivity disorder.

Other more specific methods of this invention include the above methods wherein the disorder or condition that is being treated is a sleep disorder (e.g., sleep apnea, insomnia, somnambulism, sleep deprivation, REM sleep disorders, hypersomnia, parasomnias, sleep-wake cycle disorders, narcolepsy, sleep disorders associated with shift work or irregular work schedules, and other sleep disorders).

Another practice of the invention relates to a method of treating a disorder or condition selected from the group consisting of pain resulting from soft tissue and peripheral damage, such as acute trauma; postherapeutic neuralgia, trigeminal neuralgia, segmental or intercostal neuralgia and other neuralgias; pain associated with osteoarthritis and rheumatoid arthritis; musculo-skeletal pain, such as pain experienced after trauma; spinal pain, dental pain, myofascial pain syndromes, episiotomy pain, and pain resulting from burns; deep and visceral pain, such as heart pain, muscle pain, eye pain, orofacial pain, for example, odontalgia, abdominal pain, gynaecological pain, for example, dysmenorrhoea, labour pain and pain associated with endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, neuropathic lower back pain, HIV related neuropathic pain, diabetic neuropathic pain, and arachnoiditis; neuropathic and non-neuropathic pain associated with carcinoma, often referred to as cancer pain; central nervous system pain, such as pain due to spinal cord or brain stem damage; lower back pain; sciatica; phantom limb pain, headache, including migraine and other vascular headaches, acute or chronic tension headache, cluster headache, temperomandibular pain and maxillary sinus pain; pain resulting from ankylosing spondylitis and gout; pain caused by increased bladder contractions; post operative pain; scar pain; and chronic non-neuropathic pain such as pain associated with fibromyalgia, HIV, rheumatoid and osteoarthritis, anthralgia and myalgia, sprains, strains and trauma such as broken bones; and post surgical pain in a mammal, including a human, comprising administering to said mammal an amount of a compound of Formula I as defined above, or a pharmaceutically acceptable salt or solvate thereof, wherein the amount of said compound of Formula I is effective in (1) antagonizing an NK-1 and/or NK-3 receptor, and/or (2) treating said condition or disorder.

Another practice of the invention relates to a method of treating a disorder or condition selected from the group consisting of pain resulting from soft tissue and peripheral damage, such as acute trauma; postherapeutic neuralgia, trigeminal neuralgia, segmental or intercostal neuralgia and other neuralgias; pain associated with osteoarthritis and rheumatoid arthritis; musculo-skeletal pain, such as pain experienced after trauma; spinal pain, dental pain, myofascial pain syndromes, episiotomy pain, and pain resulting from burns; deep and visceral pain, such as heart pain, muscle pain, eye pain, orofacial pain, for example, odontalgia, abdominal pain, gynaecological pain, for example, dysmenorrhoea, labour pain and pain associated with endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, neuropathic lower back pain, HIV related neuropathic pain, diabetic neuropathic pain, and arachnoiditis; neuropathic and non-neuropathic pain associated with carcinoma, often referred to as cancer pain; central nervous system pain, such as pain due to spinal cord or brain stem damage; lower back pain; sciatica; phantom limb pain, headache, including migraine and other vascular headaches, acute or chronic tension headache, cluster headache, temperomandibular pain and maxillary sinus pain; pain resulting from ankylosing spondylitis and gout; pain caused by increased bladder contractions; post operative pain; scar pain; and chronic non-neuropathic pain such as pain associated with fibromyalgia, HIV, rheumatoid and osteoarthritis, anthralgia and myalgia, sprains, strains and trauma such as broken bones; and post surgical pain in a mammal, including a human, comprising administering to said mammal an amount of a compound of Formula I, as defined above, or a pharmaceutically acceptable salt or solvate thereof, wherein the amount of said compound of Formula I is effective in (1) antagonizing an NK-1 and/or NK-3 receptor, and/or (2) treating said condition or disorder.

Other more specific methods of this invention include the above methods wherein the disorder or condition that is being treated is neuropathic pain.

Other more specific methods of this invention include the above methods wherein the disorder or condition that is being treated is HIV related neuralgia.

Other more specific methods of this invention include the above methods wherein the disorder or condition that is being treated is pain associated with fibromyalgia.

Other more specific methods of this invention include the above methods wherein the disorder or condition that is being treated is neuropathic lower back pain, HIV related neuropathic pain, diabetic neuropathic pain, arachnoiditis or neuropathic and non-neuropathic pain associated with carcinoma.

Specific preferred methods of this invention include the above methods wherein the compound of Formula I that is employed in such method is one or more of the following NK-1 antagonists and/or NK-3 antagonists:
4S-(3,5-bis-trifluoromethyl-benzloxy)-3R-2-tolyl-piperidine
4-(3,5-bis-trifluoromethyl-benzloxy)-3-phenyl-piperidine
4-(3,5-bis-trifluoromethyl-benzyloxy)-3-(4-fluoro-phenyl)-piperidine
4-(3,5-bis-trifluoromethyl-benzyloxy)-3-(2-methyl-4-fluoro-phenyl)-piperidine
4-(3,5-bis-trifluoromethyl-benzyloxy)-3-(3,4-difluoro-phenyl)-piperidine
4-(3,5-bis-trifluoromethyl-benzyloxy)-3-(2-pyridyl)-piperidine
4-(3,5-bis-trifluoromethyl-benzyloxy)-3-(3-chloro-phenyl)-piperidine
4-(4-bromo-benzyloxy)-3-(2-methyl-4-fluoro-phenyl)-piperidine
4-(3-bromo-benzyloxy)-3-(2-methyl-4-fluoro-phenyl)-piperidine
4-(2-bromo-benzyloxy)-3-(2-methyl-4-fluoro-phenyl)-piperidine
4-(2,6-dichloro-benzyloxy)-3-(2-methyl-4-fluoro-phenyl)-piperidine
4-(2,5-dichloro-benzyloxy)-3-(2-methyl-4-fluoro-phenyl)-piperidine
4-(3,5-dichloro-benzyloxy)-3-(2-methyl-4-fluoro-phenyl)-piperidine
4-(4-fluoro-benzyloxy)-3-(2-methyl-4-fluoro-phenyl)-piperidine
4-(2-fluoro-benzyloxy)-3-(2-methyl-4-fluoro-phenyl)-piperidine:
4-(3-fluoro-benzyloxy)-3-(2-methyl-4-fluoro-phenyl)-piperidine
4-(3,4-difluoro-benzyloxy)-3-(2-methyl-4-fluoro-phenyl)-piperidine
4-(3,4-difluoro-benzyloxy)-3-(2-methyl-4-fluoro-phenyl)-piperidine
4-(2,6-di-fluoro-benzyloxy)-3-(2-methyl-4-fluoro-phenyl)-piperidine
4-(3,6-di-fluoro-benzyloxy)-3-(2-methyl-4-fluoro-phenyl)-piperidine
4-(2,4,6-tri-fluoro-benzyloxy)-3-(2-methyl-4-fluoro-phenyl)-piperidine
4-(2,3,6-tri-fluoro-benzyloxy)-3-(2-methyl-4-fluoro-phenyl)-piperidine
4-(4-trifluoromethyl-benzyloxy)-3-(2-methyl-4-fluoro-phenyl)-piperidine
4-(3-trifluoromethyl-benzyloxy)-3-(2-methyl-4-fluoro-phenyl)-piperidine
4-(3,5-bis-trifluoromethyl-benzyloxy)-3-(2-methyl-4-fluoro-phenyl)-piperidine
4-(2,4-bis-trifluoromethyl-benzyloxy)-3-(2-methyl-4-fluoro-phenyl)-piperidine
4-(4-trifluoromethoxyl-benzyloxy)-3-(2-methyl-4-fluoro-phenyl)-piperidine
4-(2-methyl-3,4-Bisfluoromethyl-benzyloxy)-3-(2-methyl-4-fluoro-phenyl)-piperidine
4-(2-methyl-3,5-bis-fluoromethyl-benzyloxy)-3-(2-methyl-4-fluoro-phenyl)-piperidine
4-(2-ethyl-3,5-bis-fluoromethyl-benzyloxy)-3-(2-methyl-4-fluoro-phenyl)-piperidine
4-(2-methyl-benzyloxy)-3-(2-methyl-4-fluoro-phenyl)-piperidine
4-(2-chloro-5-methoxy-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid methylester
4-(3-methoxy-6-bromo-benzyloxy)-3-(2-methyl-4-fluoro-phenyl)-piperidine
4-(3-iodo-4-chloro-benzyloxy)-3-(2-methyl-4-fluoro-phenyl)-piperidine
4-(biphenyl-3-ylmethoxy)-3-(2-methyl-4-fluoro-phenyl)-piperidine
4-[2-(4-fluoro-benzyl)-benzyloxy]-3-(4-fluoro-2-methyl-phenyl)-piperidine
4-(3-methyl-benzyloxy)-3-(2-methyl-4-fluoro-phenyl)-piperidine
4-(2-trifluoromethyl-benzyloxy)-3-(2-methyl-4-fluoro-phenyl)-piperidine
4-(3-4-dimethyl-benzyloxy)-3-(2-methyl-4-fluoro-phenyl)-piperidine
4-(3-methyl-5-fluoro-benzyloxy)-3-(2-methyl-4-fluoro-phenyl)-piperidine
4-(2-methyl-3-fluoro-benzyloxy)-3-(2-methyl-4-fluoro-phenyl)-piperidine
4-(2,6-dibromo-benzyloxy)-3-(2-methyl-4-fluoro-phenyl)-piperidine
4-(3-chloro-6-methyl-benzyloxy)-3-(2-methyl-4-fluoro-phenyl)-piperidine
4-(2-iodo-benzyloxy)-3-(2-methyl-4-fluoro-phenyl)-piperidine
4-(2-isopropyl-benzyloxy)-3-(2-methyl-4-fluoro-phenyl)-piperidine
4-(3-fluoro-5-methyl-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid ethylester
4-benzyloxy-3-(2-methyl-4-fluoro-phenyl)-piperidine
3-[3-(2-methyl-4-fluoro-phenyl)]-piperidine-4-yloxymethyl-pyridine
3-[3-(2-methyl-4-fluoro-phenyl)]-piperidine-4-yloxymethyl-benzonitrile
1-[4S-(3,5-bis-trifluoromethyl-benzyloxy)-3R-2-tolyl-piperidine-1-yl]-2-(4-acetyl-piperazine-1-yl)-ethanone
1-[4S-(3,5-bis-trifluoromethyl-benzyloxy)-3R-2-tolyl-piperidine-1-yl]-2-pyrrolidin-1-yl-ethanone
1-[4-(2-ethyl-3,5-difluorobenzyloxy)-3-(4-fluoro-2-methyl-phenyl)-piperidine] 2-pyrrolidin-1-yl-ethanone
1-[4-(3,5-dichlorobenzyloxy)-3-(4-fluoro-2-methyl-phenyl)-piperidine] 2-pyrrolidin-1-yl-ethanone
1-[4-(3-methyl-5-fluorobenzyloxy)-3-(4-fluoro-2-methyl-phenyl)piperidine] 2-pyrrolidin-1-yl-ethanone
1-[4-(3-iodo-4-chlorobenzyloxy)-3-(4-fluoro-2-methyl-phenyl)piperidine] 2-pyrrolidin-1-yl-ethanone:
1-[4-(4,5-dichlorobenzyloxy)-3-(4-fluoro-2-methyl-phenyl)piperidine] 2-pyrrolidin-1-yl-ethanone
1-[4-(2-trifluoromethylbenzyloxy)-3-(4-fluoro-2-methyl-phenyl)piperidine] 2-pyrrolidin-1-yl-ethanone
1-[4-(5-iodobenzyloxy)-3-(4-fluoro-2-methyl-phenyl)piperidine] 2-pyrrolidin-1-yl-ethanone
1-[4-(biphenyl-2-ylmethoxy)-3-(4-fluoro-2-methyl-phenyl)piperidine] 2-pyrrolidin-1-yl-ethanone:
1-[4-(2-chloro-5-methoxybenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine]
2-pyrrolidin-1-yl-ethanone
1-[4-(4-iodobenzyloxy)-3-(4-fluoro-2-methyl-phenyl)piperidine] 2-pyrrolidin-1-yl-ethanone
1-[4-(5-bromobenzyloxy)-3-(4-fluoro-2-methyl-phenyl)piperidine]
2-pyrrolidin-1-yl-ethanone
1-[4-(2-methyl-5-fluorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2-iodobenzyloxy)-3-(4-fluoro-2-methyl-phenyl)piperidine] 2-pyrrolidin-1-yl-ethanone
1-[4-(2-difluoromethoxybenzyloxy)-3-(4-fluoro-2-methyl-phenyl)piperidine] 2-pyrrolidin-1-yl-ethanone:
1-[4-(2-methyl-5-chlorobenzyloxy)-3-(4-fluoro-2-methyl-phenyl)piperidine]
2-pyrrolidin-1-yl-ethanone
1-[4-(3-methylbenzyloxy)-3-(4-fluoro-2-methyl-phenyl)piperidine] 2-pyrrolidin-1-yl-ethanone
1-[4-(2-methyl-3,5-difluorobenzyloxy)-3-(4-fluoro-2-methyl-phenyl)piperidine] 2-pyrrolidin-1-yl-ethanone
1-[4-(3-methoxybenzyloxy)-3-(4-fluoro-2-methyl-phenyl)piperidine] 2-pyrrolidin-1-yl-ethanone
1-[4-(3-chlorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2,5-dichlorobenzyloxy)-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(4-bromobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2-methyl-3,4-difluorobenzyloxy)-3-(4-fluoro-2-methyl-phenyl)piperidine] 2-pyrrolidin-1-yl-ethanone
1-[4-(2-chlorobenzyloxy)-3-(4-fluoro-2-methyl-phenyl)piperidine] 2-pyrrolidin-1-yl-ethanone
1-[4-(2-methyl-3-fluorobenzyloxy)-3-(4-fluoro-2-methyl-phenyl)piperidine] 2-pyrrolidin-1-yl-ethanone
1-[4-(4-trifluoromethoxybenzyloxy)-3-(4-fluoro-2-methyl-phenyl)piperidine] 2-pyrrolidin-1-yl-ethanone
1-[4-(4-trifluoromethylbenzyloxy)-3-(4-fluoro-2-methyl-phenyl)piperidine] 2-pyrrolidin-1-yl-ethanone
1-[4-(2-fluorobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(2-cyanobenzyloxy)-3-(4-fluoro-2-methyl-phenyl)piperidine] 2-pyrrolidin-1-yl-ethanone
1-[4-(2,4,6-trifluorobenzyloxy)-3-(4-fluoro-2-methyl-phenyl)piperidine] 2-pyrrolidin-1-yl-ethanone
1-[4-(3-cyanobenzyloxy)-3-(4-fluoro-2-methyl-phenyl)phenyl] 2-pyrrolidin-1-yl-ethanone
1-[4-(5-methyl-6-fluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2,6-dichlorobenzyloxy)-3-(4-fluoro-2-methyl-phenyl)piperidine] 2-pyrrolidin-1-yl-ethanone
1-[4-(2,5,6-trifluorobenzyloxy)-3-(4-fluoro-2-methyl-phenyl)piperidine] 2-pyrrolidin-1-yl-ethanone
1-[4-(2,6-difluorobenzyloxy)-3-(4-fluoro-2-methyl-phenyl)piperidine] 2-pyrrolidin-1-yl-ethanone
1-[4-(benzyloxy)-3-(4-fluoro-2-methyl-phenyl)piperidine]-2-pyrrolidin-1-yl-ethanone
1-[(4-fluorophenoxy) benzyloxy-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(3-trifluoromethylbenzyloxy)-3-(4-fluoro-2-methyl-phenyl)piperidine] 2-pyrrolidin-1-yl-ethanone
1-[(4-benzoyl-benzyloxy)-3-(4-fluoro-2-methyl-phenyl)piperidine] 2-pyrrolidin-1-yl-ethanone
1-[4S-(2,4-bis-trifluoromethyl-benzyloxy)-3R-2-tolyl-piperidine-1-yl]-2-piperidine-1-yl-ethanone
1-[4S-(3,5-bis-trifluoromethyl-benzyloxy)-3R-2-tolyl-piperidine-1-yl]-2-morpholin-1-yl-ethanone
1-[4S-(3,5-bis-trifluoromethyl-benzyloxy)-3R-2-tolyl-piperidine-1-yl]-2-piperazine-1-yl-ethanone
1-[4S-(3,5-bis-trifluoromethyl-benzyloxy)-3R-2-tolyl-piperidine-1-yl]-2-(4-methyl-piperazine-1-yl)-ethanone
1-[4S-(3,5-bis-trifluoromethyl-benzyloxy)-3R-2-tolyl-piperidine-1-yl]-2-(4-ethyl-piperazine-1-yl)-ethanone
1-[4S-(3,5-bis-trifluoromethyl-benzyloxy)-3R-2-tolyl-piperidine-1-yl]-2-(4-benzyl-piperazine-1-yl)-ethanone
1-[4S-(3,5-bis-trifluoromethyl-benzyloxy)-3R-2-tolyl-piperidine-1-yl]-2-piperidine-1-carboxylic acid tert-butyl ester-1-yl-ethanone
1-[4S-(3,5-bis-trifluoromethyl-benzyloxy)-3R-2-tolyl-piperidine-1-yl]-2-piperidine-4-yl-ethanone
1-[4S-(3,5-bis-trifluoromethyl-benzyloxy)-3R-2-tolyl-piperidine-1-yl]-2-(1-Acetyl-piperidine-4-yl)-ethanone
1-[4S-(3,5-bis-trifluoromethyl-benzyloxy)-3R-2-tolyl-piperidine-1-yl]-2-(1-H-imidazole-4-yl)-ethanone
1-[4S-(3,5-bis-trifluoromethyl-benzyloxy)-3R-2-tolyl-piperidine-1-yl]-2-Pyridine-4-yl-ethanone
1-[4S-(3,5-bis-trifluoromethyl-benzyloxy)-3R-2-tolyl-piperidine-1-yl]-2-pyrrolidin-2-one-1-yl-ethanone
1-[4S-(3,5-bis-trifluoromethyl-benzyloxy)-3R-2-tolyl-piperidine-1-yl]-2-dimethylamino-1-yl-ethanone
[4S-(3,5-bis-trifluoromethyl-benzyloxy)-3R-2-tolyl-piperidine-1-yl]-(4-methyl-piperazine-1-yl)-methanone
[4S-(3,5-bis-trifluoromethyl-benzyloxy)-3R-2-tolyl-piperidine-1-yl]-piperidine-4-yl-methanone
[4S-(3,5-bis-trifluoromethyl-benzyloxy)-3R-2-tolyl-piperidine-1-yl]-piperidine-2-yl-methanone
[4S-(3,5-bis-trifluoromethyl-benzyloxy)-3R-2-tolyl-piperidine-1-yl]-thiazolidin-4-yl-methanone
[4-(3,5-bis-trifluoromethyl-benzyloxy)-3R-2-tolyl-piperidine-1-yl]-(2-hydroxy-pyridine-3-yl)-methanone
[4-(3,5-bis-trifluoromethyl-benzyloxy)-3R-2-tolyl-piperidine-1-yl]-(3-hydroxy-pyridine-2-yl)-methanone
1 -2-[4S-(3,5-bis-trifluoromethyl-benzyloxy)-3R-2-tolyl-piperidine-1-carbonyl]-4R-hydroxy-pyrrolidine-1-yl)}-ethanone
[4S-(3,5-bis-trifluoromethyl-benzyloxy)-3R-2-tolyl-piperidine-1-yl]-pyridine-4-yl-methanone
1-{2-[4S-(3,5-bis-trifluoromethyl-benzyloxy)-3R-2-tolyl-piperidine-1-carbonyl]-pyrrolidin-1-yl)}-ethanone
[4S-(3,5-bis-trifluoromethyl-benzyloxy)-3R-2-tolyl-piperidine-1-yl]-pyrrolidin-1-yl-methanone
[4S-(3,5-bis-trifluoromethyl-benzyloxy)-3R-2-tolyl-piperidine-1-yl]-morpholin-4-yl-methanone
[4S-(3,5-bis-trifluoromethyl-benzyloxy)-3R-2-tolyl-piperidine-1-yl]-2-dimethylamino-ethanone
N-{2-[4S-(3,5-bis-trifluoromethyl-benzyloxy)-3R-2-tolyl-piperidine-1-yl]-2oxo-ethyl}-acetamide
2-amino-1-[4S-(3,5-bis-trifluoromethyl-benzyloxy)-3R-2-tolyl-piperidine-1-yl]-ethanone
2-[4S-(3,5-bis-trifluoromethyl-benzyloxy)-3R-2-tolyl-piperidine-1-yl]-N-N-dimethyl-acetamide
4-[4S-(3,5-bis-trifluoromethyl-benzyloxy)-3R-2-tolyl-piperidine-1-carbonyl]-piperidine-2,6-dione
[4S-(3,5-bis-trifluoromethyl-benzyloxy)-3R-2-tolyl-piperidine-1-yl]-pyrrolidin-2-yl-methanone
[4S-(3,5-bis-trifluoromethyl-benzyloxy)-3R-2-tolyl-piperidine-1-yl]-piperazine-2-yl-methanone
5-[4S-(3,5-bis-trifluoromethyl-benzyloxy)-3R-2-tolyl-piperidine-1-methyl]-2-4-dihydro-[1,2,4] triazol-3-one
5-[4S-(3,5-bis-trifluoromethyl-benzyloxy)-3R-(3,4-difluoro-phenyl)-2-piperidine-1-methyl]-2-4-dihydro-[1,2,4] triazol-3-one
5-[4S-(3,5-bis-trifluoromethyl-benzyloxy)-3R-phenyl-2-piperidine-1-methyl]-2-4-dihydro-[1,2,4] triazol-3-one
5-[4S-(3,5-dimethyl-benzyloxy)-3R-phenyl-2-piperidine-1-methyl]-24-dihydro-[1,2,4] triazol-3-one
5-[4-(3,5-bis-trifluoromethyl-benzyloxy)-3-(4-fluoro-phenyl)-2-piperidine-1-methyl]-2-4-dihydro-[1,2,4] triazol-3-one
5-[4-(3,5-bis-fluoromethyl-benzyloxy)-3-phenyl)-2-piperidine-1-methyl]-2-4-dihydro-[1,2,4] triazol-3-one
4-(3,5-bis-trifluoromethyl-benzloxy)-1-methyl-3-tolyl-piperidine
1-[4-(3,5-bis-trifluoromethyl-benzloxy)-3-(4-fluoro-2-methyl-phenyl)-piperidine-1-yl]-ethanone
1-[4S-(3,5-bis-trifluoromethyl-benzyloxy)-3R-2-tolyl-piperidine-1-yl]-ethanone
{5-[4-(3,5-bis-trifluoromethyl-benzyloxy)-3-phenyl-piperidine-1-yl]-2H[1,2,3] triazol-4-methyl}dimethyl-amine
{5-[4-(3,5-bis-trifluoromethyl-benzyloxy)-3-(4-fluoro-phenyl)-piperidine-1-yl]-2H[1,2,3] triazol-4-methyl} dimethyl-amine
{5-[4-(3,5-dimethyl-benzyloxy)-3-phenyl-piperidine-1-yl]-2H[1,2,3] triazol-4-methyl}dimethyl-amine
3-(4-fluoro-2-methyl-phenyl)-4-(2-phenyl-butyrylamino)-piperidine
4-oxo-2,4-diphenyl-(3-phenyl-piperidin-4-yl)-butyramide
2-(4-nitro-phenyl) -(3-phenyl-piperidin-4-yl)-propionamide
2-phenyl-(3-phenyl-piperidin-4-yl)-propionamide
1,2,3,4-tetrahydro-naphthalene-1-carboxylic acid (3-phenyl-piperidin-4-yl)-amide:
3-methyl-2-phenyl-(3-phenyl-piperidin-4-yl)-butyramide
2-phenyl-(3-phenyl-piperidin-4-yl)-butyramide
2-(3-benzoyl-phenyl)-(3-phenyl-piperidin-4-yl)-propionamide
(3-phenyl-piperidin-4-yl)-2-tolyl-acetamide
(3-phenyl-piperidin-4-yl)-2-[4-(thiophene-2-carbonyl)-phenyl]-propionamide
6-fluoro-2-oxo-1,2,3,4-tetrahydro-quinoline-4-carboxylic acid (3-phenyl-piperidin-4-yl)-amide
3-furan-2-yl-2-phenyl-(3-phenyl-piperidin-4-yl)-propionamide
6-chloro-8-methyl-chroman-4-carboxylic acid (3-phenyl-piperidin-4-yl)-amide
5-cyclohexyl-indan-1-carboxylic acid (3-phenyl-piperidin-4-yl)-amide
2-(3,4-dimethoxy-phenyl)-hexanoic acid (3-phenyl-piperidin-4-yl)-amide
6-methyl-indan-1-carboxylic acid (3-phenyl-piperidin-4-yl)-amide
2-[3-chloro-4-(2,5-dihydro-pyrrol-1-yl)-phenyl-(3-phenyl-piperidin-4-yl)-propionamide
6-methoxy-3-oxo-indan-1-carboxylic acid (3-phenyl-piperidin-4-yl)-amide
6,7-dichloro-chroman-4-carboxylic acid (3-phenyl-piperidin-4-yl)-amide
2-{4-[2-(4-methoxy-phenyl)-vinyl]-phenyl-(3-phenyl-piperidin-4-yl)}-propionamide:
2-phenyl-(3-phenyl-piperidin-4-yl)-propionamide
2-(4-chloro-phenyl-3-phenyl-piperidin-4-yl)-propionamide
2-phenyl-hexanoic acid (3-phenyl-piperidin-4-yl)-amide
thiochroman-4-carboxylic acid (3-phenyl-piperidin-4-yl)-amide
5-methoxy-1,2,3,4-tetrahydro-naphthalene-1-carboxylic acid (3-phenyl-piperidin-4-yl)-amide
1-oxo-3-phenyl-1,2,3,4-tetrahydro-isoquinoline-4-carboxylic acid (3-phenyl-piperidin-4-yl)-amide
6-methoxy-2-methyl-1,2,3,4-tetrahydro-isoquinoline-4-carboxylic acid (3-phenyl-piperidin-4-yl)-amide
2-(4-hydroxy-phenyl)-3-phenyl-3-phenyl-piperidin-4-yl)-propionamide:
2-(2-fluoro-biphenyl-4-yl-3-phenyl-piperidin-4-yl)-propionamide
chroman-2-carboxylic acid (3-phenyl-piperidin-4-yl)-amide
2,4-diphenyl-3-phenyl-piperidin-4-yl)-butyramide
6-chloro-thiochroman-4-carboxylic acid (3-phenyl-piperidin-4-yl)-amide
7-methoxy-chroman-4-carboxylic acid (3-phenyl-piperidin-4-yl)-amide
2-[4-(2-hydroxy-2-methyl-propyl)-phenyl]-(3-phenyl-piperidin-4-yl)-propionamide:
2-phenyl-(3-phenyl-piperidin-4-yl)-butyramide
2-(4-hydroxy-phenyl-3-phenyl-piperidin-4-yl)-propionamide
2-phenyl-3-phenyl-piperidin-4-yl)-acetamide
2,3-diphenyl-(3-phenyl-piperidin-4-yl)-propionamide
2-(3-phenoxy-phenyl-3-phenyl-piperidin-4-yl)-propionamide
2-(4-isobutyl-phenyl-3-phenyl-piperidin-4-yl)-propionamide
2-phenyl-3-phenyl-piperidin-4-yl)-propionamide
indan-1-carboxylic acid (3-phenyl-piperidin-4-yl)-amide
2-phenoxy -(3-phenyl-piperidin-4-yl)-propionamide
3-(4-methoxy-phenyl)-2-phenyl-(3-phenyl-piperidin-4-yl)-propionamide
2-cyclopentyl-2-phenyl-(3-phenyl-piperidin-4-yl)-acetamide
1,2,3,4-tetrahydro-isoquinoline-4-carboxylic acid (3-phenyl-piperidin-4-yl)-amide
2-phenyl-3-(5-phenyl-furan-2-yl-3-phenyl-piperidin-4-yl)-propionamide
3-(4-hydroxy-phenyl)-2-phenyl-3-phenyl-piperidin-4-yl)-propionamide
6,7-dichloro-thiochroman-4-carboxylic acid (3-phenyl-piperidin-4-yl)-amide
6-fluoro-3-hydroxy-indan-1-carboxylic acid (3-phenyl-piperidin-4-yl)-amide
4,5,6,7-tetramethyl-3-oxo-indan-1-carboxylic acid (3-phenyl-piperidin-4-yl)-amide
2-(2,5-dimethyl-phenyl)-6-phenyl-hexanoic acid (3-phenyl-piperidin-4-yl)-amide
3-methyl-2-phenyl-pentanoic acid (3-phenyl-piperidin-4-yl)-amide
(3-phenyl-piperidin-4-yl)-2-tolyl-butyramide
6-fluoro-chroman-4-carboxylic acid (3-phenyl-piperidin-4-yl)-amide
7-methoxy-1,2,3,4-tetrahydro-naphthalene-1-carboxylic acid (3-phenyl-piperidin-4-yl)-amide
3-oxo-indan-1-carboxylic acid (3-phenyl-piperidin-4-yl)-amide
2-biphenyl-4-yl-pent-4-enoic acid (3-phenyl-piperidin-4-yl)-amide
2-naphthalen-1-yl-heptanoic acid (3-phenyl-piperidin-4-yl)-amide
2-(6-methoxy-naphthalen-2-yl-3-phenyl-piperidin-4-yl)-propionamide
2-(4-chloro-phenyl)-3-methyl-3-phenyl-piperidin-4-yl)-butyramide
5-methyl-2-tolyl-hexanoic acid (3-phenyl-piperidin-4-yl)-amide
6-methoxy-1,2,3,4-tetrahydro-naphthalene-1-carboxylic acid (3-phenyl-piperidin-4-yl)-amide
6-methoxy-chroman-4-carboxylic acid (3-phenyl-piperidin-4-yl)-amide
6-fluoro-3-oxo-indan-1-carboxylic acid (3-phenyl-piperidin-4-yl)-amide
3-hydroxy-2-phenyl-(3-phenyl-piperidin-4-yl)-propionamide
4-(4-methoxy-phenyl)-4-oxo-2-phenyl-(3-phenyl-piperidin-4-yl)-butyramide
6-chloro-9-methyl-2,3,4,9-tetrahydro-1-carbazole-4-carboxylic acid (3-phenyl-piperidin-4-yl)-amide
2-(4-isobutyl-phenyl-3-phenyl-piperidin-4-yl)-propionamide
2-phenoxy-3-phenyl-piperidin-4-yl)-butyramide
2-biphenyl-4-yl-hex-4-enoic acid (3-phenyl-piperidin-4-yl)-amide
2-cyclohex-2-enyl-2-phenyl-3-phenyl-piperidin-4-yl)-acetamide
6,7-dimethyl-chroman-4-carboxylic acid (3-phenyl-piperidin-4-yl)-amide
2-[2-(4-chloro-phenyl)-benzooxazol-5-yl-3-phenyl-piperidin-4-yl]-propionamide
3-(4-hydroxy-3,5-diiodo-phenyl)-2-phenyl-3-phenyl-piperidin-4-yl)-propionamide
2-(4-methoxy-phenyl)-3-(5-phenyl-furan-2-yl)-(3-phenyl-piperidin-4-yl)-propionamide
6-chloro-chroman-4-carboxylic acid (3-phenyl-piperidin-4-yl)-amide
4,5-dimethoxy-indan-1-carboxylic acid (3-phenyl-piperidin-4-yl)-amide
6,7-dichloro-2-methyl-chroman-4-carboxylic acid (3-phenyl-piperidin-4-yl)-amide
2-(6-hydroxy-naphthalen-2-yl-3-phenyl-piperidin-4-yl)-propionamide
   or pharmaceutically acceptable salts or solvates thereof.

Other more specific methods of this invention include the above methods wherein the compound of Formula I is administered to a human for the treatment of major depressive disorder and concomitant generalized anxiety disorder.

Other more specific methods of this invention include the above methods wherein the compound of Formula I is administered to a human for the treatment of major depressive disorder and concomitant irritable bowel syndrome.

Other more specific methods of this invention include the above methods wherein the compound of Formula I is administered to a human for the treatment of major depressive disorder and concomitant functional abdominal pain.

Other more specific methods of this invention include the above methods wherein the compound of Formula I is administered to a human for the treatment of major depressive disorder and concomitant neuropathic pain.

Other more specific methods of this invention include the above methods wherein the compound of Formula I is administered to a human for the treatment of major depressive disorder and concomitant premenstrual dysphoric disorder.

Other more specific methods of this invention include the above methods wherein the compound of Formula I is administered to a human for the treatment of major depressive disorder and concomitant dysthymia.

Other more specific methods of this invention include the above methods wherein the compound of Formula I is administered to a human for the treatment of major depressive disorder and concomitant fibromyalgia.

Other more specific methods of this invention include the above methods wherein the compound of Formula I is administered to a human for the treatment of major depressive disorder and a concomitant somatoform disorder such as somatization disorder, conversion disorder, body dysmorphic disorder, hypochondriasis, somatoform pain disorder or undifferentiated somatoform disorder.

Other more specific methods of this invention include the above methods wherein the compound of Formula I is administered to a human for the treatment of generalized anxiety disorder and concomitant irritable bowel syndrome.

Other more specific methods of this invention include the above methods wherein the compound of Formula I is administered to a human for the treatment of generalized anxiety disorder and concomitant functional abdominal pain.

Other more specific methods of this invention include the above methods wherein the compound of Formula I is administered to a human for the treatment of generalized anxiety disorder and concomitant neuropathic pain.

Other more specific methods of this invention include the above methods wherein the compound of Formula I is administered to a human for the treatment of generalized anxiety disorder and concomitant premenstrual dysphoric disorder.

Other more specific methods of this invention include the above methods wherein the compound of Formula I is administered to a human for the treatment of generalized anxiety disorder and concomitant dysthymia.

Other more specific methods of this invention include the above methods wherein the compound of Formula I is administered to a human for the treatment of generalized anxiety disorder and concomitant fibromyalgia.

Other more specific methods of this invention include the above methods wherein the compound of Formula I is administered to a human for the treatment of generalized anxiety disorder and a concomitant somatoform disorder selected from the group consisting of somitization disorder, conversion disorder, hypochondriasis, somatoform pain disorder (or simply “pain disorder”), body dysmorphic disorder, undifferentiated somatoform disorder, and somatoform disorder not otherwise specified. See Diagnostic and Statistical manual of Mental Disorders, Fourth Edition (DSM-IV), American Psychiatric Association, Washington, D.C., Can 1194, pp. 435-436.

Other more specific methods of this invention include the above methods wherein the compound of Formula I is administered to a human for the treatment of major depressive disorder accompanied by one or more somatic symptoms such as loss of appetite, sleep disturbances (e.g., insomnia, interrupted sleep, early morning awakening, tired awakening), loss of libido, restlessness, fatigue, constipation, dyspepsia, heart palpitations, aches and pains (e.g., headache, neck pain, back pain, limb pain, joint pain, abdominal pain), dizziness, nausea, heartburn, nervousness, tremors, burning and tingling sensations, morning stiffness, abdominal symptoms (e.g., abdominal pain, abdominal distention, gurgling, diarrhea), or the symptoms associated with generalized anxiety disorder (e.g., excessive anxiety and worry (apprehensive expectation), occurring more days than not for at least six months, about a number of events and activities, difficulty controlling the worry, etc.) See Diagnostic and Statistical manual of Mental Disorders, Fourth Edition (DSM-IV), American Psychiatric Association, Washington, D.C., Can 1194, pp. 435-436 and 445-469. This document is incorporated herein by reference in its entirety.

Other more specific methods of this invention include the above methods wherein the Formula I is administered to a human for the treatment of major depressive disorder accompanied by one or more somatic symptoms such fatigue, headache, neck pain, back pain, limb pain, joint pain, abdominal pain, abdominal distention, gurgling, diarrhea nervousness, or the symptoms associated with generalized anxiety disorder (e.g., excessive anxiety and worry (apprehensive expectation), occurring more days than not for at least six months, about a number of events and activities, difficulty controlling the worry, etc. See Diagnostic and Statistical manual of Mental Disorders, Fourth Edition (DSM-IV), American Psychiatric Association, Washington, D.C., Can 1194, pp. 435-436 and 445-469.

Other more specific methods of this invention include the above methods wherein the compound of Formula I is administered to a human for the treatment of generalized anxiety disorder accompanied by one or more somatic symptoms such as loss of appetite, sleep disturbances (e.g., insomnia, interrupted sleep, early morning awakening, tired awakening), loss of libido, restlessness, fatigue, constipation, dyspepsia, heart palpitations, aches and pains (e.g., headache, neck pain, back pain, limb pain, joint pain, abdominal pain), dizziness, nausea, heartburn, nervousness, tremors, burning and tingling sensations, morning stiffness, abdominal symptoms (e.g., abdominal pain, abdominal distention, gurgling, diarrhea), or the symptoms associated with major depressive disorder (e.g., sadness, tearfulness, loss of interest, fearfulness, helplessness, hopelessness, fatigue, low self esteem, obsessive ruminations, suicidal thoughts, impaired memory and concentration, loss of motivation, paralysis of will, reduced appetite, increased appetite).

Other more specific methods of this invention include the above methods wherein the compound of Formula I is administered to a human for the treatment of generalized anxiety disorder accompanied by one or more somatic symptoms such as fatigue, headache, neck pain, back pain, limb pain, joint pain, abdominal pain, abdominal distention, gurgling, diarrhea nervousness, or the symptoms associated with major depressive disorder (e.g., sadness, tearfulness, loss of interest, fearfulness, helplessness, hopelessness, low self esteem, obsessive ruminations, suicidal thoughts, fatigue, impaired memory and concentration, loss of motivation, paralysis of will, reduced appetite, increased appetite).

The present invention also includes isotopically labelled compounds, which are identical to those recited in Formula I compounds, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, 35S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, can be preferred in some circumstances. Isotopically labelled compounds of Formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

In another practice, the compound of Formula I can be used in conjunction with one or more other therapeutic agents, e.g. different antidepressant agents such as tricyclic antidepressants (e.g. amitriptyline, dothiepin, doxepin, trimipramine, butripyline, clomipramine, desipramine, imipramine, iprindole, lofepramine, nortriptyline or protriptyline), monoamine oxidase inhibitors (e.g. isocarboxazid, pheneizine or tranylcyclopramine) or 5-HT re-uptake inhibitors (e.g. fluvoxamine, sertraline, fluoxetine or paroxetine), and/or with antiparkinsonian agents such as dopaminergic antiparkinsonian agents (e.g. levodopa, preferably in combination with a peripheral decarboxylase inhibitor e.g. benserazide or carbidopa, or with a dopamine agonist e.g., bromocriptine, lysuride or pergolide).

In a preferred practice, the compound of Formula I is used in combination with a 5-HT re-uptake inhibitor (e.g. fluvoxamine, sertraline, fluoxetine or paroxetine), preferably sertraline (or a pharmaceutically acceptable salt or polymorph thereof as would be understood by the artisan) as psychotherapeutics and can be used in the treatment or prevention of disorders the treatment or prevention of which is facilitated by modulating serotonergic neurotransmission such as hypertension, depression (e.g. depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g. agoraphobia, social phobia and simple phobias), posttraumatic stress syndrome, avoidant personality disorder, premature ejaculation, eating disorders (e.g. anorexia nervosa and bulimia nervosa), obesity, chemical dependencies (e.g. addictions to alcohol, cocaine, heroin, phenobarbital, nicotine and benzodiazepines), cluster headache, migraine, pain, Alzheimer's disease, obsessive-compulsive disorder, panic disorder, memory disorders (e.g. dementia, amnestic disorders, and age-related cognitive decline (ARCD)), Parkinson's diseases (e.g. dementia in Parkinson's disease, neuroleptic-induced parkinsonism and tardive dyskinesias), endocrine disorders (e.g. hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania and headache (associated with vascular disorders).

Sertraline, (1S-cis)-4-(3,4-dichlorophenyl)-1,2,3,4-tetrahydro-N-methyl-1-naphthalenamine, has the chemical formula C₁₇H₁₇NC₁₂; its synthesis is described in U.S. Pat. No. 4,536,518 incorporated herein by reference. Sertraline hydrochloride is useful as an antidepressant and anorectic agent, and is also useful in the treatment of depression, chemical dependencies, anxiety obsessive compulsive disorders, phobias, panic disorder, post traumatic stress disorder, and premature ejaculation.

### Administration

The compound of the invention can be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can be readily administered in a variety of dosage forms such as tablets, powders, lozenges, liquid preparations, syrups, injectable solutions and the like. These pharmaceutical compositions can optionally contain additional ingredients such as flavorings, binders, excipients and the like.

Thus the compound of the invention can be formulated for oral, buccal, intranasal, parenteral (e.g. intravenous, intramuscular or subcutaneous), transdermal (e.g. patch) or rectal administration or in a form suitable for administration by inhalation or insufflation. E.g. for oral administration, the pharmaceutical compositions can take the form of tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets can be coated by methods known in the art. Liquid preparations for oral administration can take the form of e.g. solutions, syrups or suspensions, or they can be presented as a dry product for constitution with water or other suitable vehicle before use. Such liquid preparations can be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid). For buccal administration, the composition can take the form of tablets or lozenges formulated in conventional manner.

The compound of the invention can be formulated for parenteral administration by injection, including using conventional catheterization techniques or infusion. Formulations for injection can be presented in unit dosage form, e.g. in ampules or in multi-dose containers, with an added preservative. They can take such forms as suspensions, solutions or emulsions in oily or aqueous vehicles, and can contain formulating agents such as suspending, stabilizing and/or dispersing agents. Alternatively, the active ingredient can be in powder form for reconstitution with a suitable vehicle, e.g. sterile pyrogen-free water, before use.

The compound of the invention can also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules and cartridges (made e.g. from gelatin) for use in an inhaler or insufflator can be formulated containing a powder mix of a compound of the invention and a suitable powder base such as lactose or starch.

A proposed dose of the active compounds of the invention for oral, parenteral or buccal administration to the average adult human for the treatment of the disorders or conditions referred to above is about 0.1 to about 200 mg of the active ingredient per unit dose which could be administered, for example, 1 to 4 times per day.

Aerosol formulations for treatment of the disorders or conditions referred to above in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration can be once or several times daily, e.g. 2, 3, 4 or 8 times, giving for example, 1, 2 or 3 doses each time.

In connection with the use of the compound of the invention with a 5-HT re-uptake inhibitor, preferably sertraline, for the treatment of subjects possessing any of the above disorders or conditions, it is to be noted that these can be administered either alone or in combination with pharmaceutically acceptable carriers by either of the routes previously indicated, and that such administration can be carried out in both single and multiple dosages. More particularly, the active combination can be administered in a wide variety of different dosage forms, i.e. they can be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of Formula I are present in such dosage forms at concentration levels ranging from about 0.5% to about 90% by weight of the total composition, i.e., in amounts which are sufficient to provide the desired unit dosage and a 5-HT re-uptake inhibitor, preferably sertraline, is present in such dosage forms at concentration levels ranging from about 0.5% to about 90% by weight of the total composition, i.e. in amounts which are sufficient to provide the desired unit dosage.

A proposed daily dose of the compound of the invention in the combination formulation (a formulation containing the compound of the invention and a 5-HT re-uptake inhibitor) for oral, parenteral, rectal or buccal administration to the average adult human for the treatment of the disorders or conditions referred to above is from about 0.01 mg to about 2000 mg, preferably from about 0.1 mg to about 200 mg of the active ingredient of Formula I per unit dose which could be administered, for example, 1 to 4 times per day.

A proposed daily dose of a 5-HT re-uptake inhibitor, preferably sertraline, in the combination formulation for oral, parenteral or buccal administration to the average adult human for the treatment of the disorders or conditions referred to above is from about 0.1 mg to about 2000 mg, preferably from about 1 mg to about 200 mg of the 5-HT re-uptake inhibitor per unit dose which could be administered, for example, 1 to 4 times per day.

A preferred dose ratio of sertraline to an active compound of this invention in the combination formulation for oral, parenteral or buccal administration to the average adult human for the treatment of the disorders or conditions referred to above is from about 0.00005 to about 20000; preferably from about 0.25 to about 2000.

Aerosol combination formulations for treatment of the disorders or conditions referred to above in the average adult human are preferably arranged so that each metered dose or “puff” of aerosol contains from about 0.01 mg to about 100 mg of the active compound of this invention, preferably from about 1 mg to about 10 mg of such compound. Administration can be once or several times daily, e.g. 2, 3, 4 or 8 times, giving for example, 1, 2 or 3 doses each time.

Aerosol formulations for treatment of the disorders or conditions referred to above in the average adult human are preferably arranged so that each metered dose or “puff” of aerosol contains from about 0.01 mg to about 2000 mg of a 5-HT re-uptake inhibitor, preferably sertraline, preferably from about 1 mg to about 200 mg of sertraline. Administration can be once or several times daily, for example 2, 3, 4 or 8 times, giving for example, 1, 2 or 3 doses each time.

As previously indicated, a 5-HT re-uptake inhibitor, preferably sertraline, in combination with compounds of Formula I are readily adapted to therapeutic use as antidepressant agents. In general, these antidepressant compositions containing a 5-HT re-uptake inhibitor, preferably sertraline, and a compound of Formula I are normally administered in dosages ranging from about 0.01 mg to about 100 mg per kg of body weight per day of a 5-HT re-uptake inhibitor, preferably sertraline, preferably from about 0.1 mg to about 10 mg per kg of body weight per day of sertraline; with from about 0.001 mg to about 100 mg per kg of body weight per day of a compound of Formula I, preferably from about 0.01 mg to about 10 mg per kg of body weight per day of a compound of Formula I, although variations will necessarily occur depending upon the disorders or conditions of the subject being treated and the particular route of administration chosen.

Additionally, it is also possible to administer the compounds of Formula I and their pharmaceutically acceptable salts topically and this can preferably be done by way of creams, jellies, gels, pastes, ointments and the like, in accordance with standard pharmaceutical practice.

### NK-1 Assays

The activity of the compounds of Formula I or their pharmaceutically acceptable salts or solvates as substance P antagonists (NK-1) can be determined by their ability to inhibit the binding of substance P at its receptor sites in bovine caudate tissue, employing radioactive ligands to visualize the tachykinin receptors by means of autoradiography. The substance P antagonizing activity of the herein described compounds can be evaluated by using the standard assay procedure described by M. A. Cascieri et al., as reported in the Journal of Biological Chemistry, Vol. 258, p. 5158 (1983). This method essentially involves determining the concentration of the individual compound required to reduce by 50% the amount of radiolabelled substance P ligands at their receptor sites in said isolated cow tissues, thereby affording characteristic IC₅₀ values for each compound tested.

In this procedure, bovine caudate tissue is removed from a −70° C. freezer and homogenized in 50 volumes (w./v.) of an ice-cold 50 mM Tris (i.e., trimethamine which is 2-amino-2-hydroxymethyl-1,3-propanediol) hydrochloride buffer having a pH of 7.7. The homogenate is centrifuged at 30,000×G for a period of 20 minutes. The pellet is resuspended in 50 volumes of Tris buffer, rehomogenized and then recentrifuged at 30,000×G for another twenty-minute period. The pellet is then resuspended in 40 volumes of ice-cold 50 mM Tris buffer (pH 7.7) containing 2 mM of calcium chloride, 2 mM of magnesium chloride, 40 g/ml of bacitracin, 4 μg/ml of leupeptin, 2 μg of chymostatin and 200 g/ml of bovine serum albumin. This step completes the production of the tissue preparation.

The radioligand binding procedure is then carried out in the following manner, viz., by initiating the reaction via the addition of 100 μl of the test compound made up to a concentration of 1 μM, followed by the addition of 100 μl of radioactive ligand made up to a final concentration 0.5 mM and then finally by the addition of 800 μl of the tissue preparation produced as described above. The final volume is thus 1.0 ml, and the reaction mixture is next vortexed and incubated at room temperature (ca. 20° C.) for a period of 20 minutes. The tubes are then filtered using a cell harvester, and the glass fiber filters (Whatman GF/B) are washed four times with 50 mM of Tris buffer (pH 7.7), with the filters having previously been presoaked for a period of two hours prior to the filtering procedure. Radioactivity is then determined in a Beta counter at 53% counting efficiency, and the IC₅₀ values are calculated by using standard statistical methods.

### NK-3 Assays

Cell Culture:

CHO cells expressing the human NK-3 receptor are passaged 2× weekly in medium containing alpha-MEM plus 10% heat inactivated GIBCO FBS and 0.8 mg/ml G418. Cells are split by lifting using D-PBS containing 5 mM EDTA and aliquotting into fresh flasks. For assay, cells are lifted as above, then spun for
10′ at 18,000 RPM in an SS34 rotor. Cells are resuspended in assay buffer (below) using a polytron at 50% max speed, and spun. Finally, cells are resuspended to a concentration of 25 mg/ml in assay buffer, and kept on ice until added to the assay.

Receptor Binding:

25 ul of cells are added to 96-well V-bottom polypropylene plates containing 200ul of 125I-MePheNKB (NEX-285, 0.1 nM final concentration) and 25 ul of buffer or test compound made in 50 mM Tris pH 7.4 with 120 mM NaCl and 1 mg/ml BSA and 20 ug/ml chymostatin, 20 ug/ml leupeptin, 0.2 mg/ml bacitracin. Plates are incubated at 4° C. After 2 hours, the plates are harvested using a 96-well Skatron harvester set for 15 second wash with 50 mM Tris HCl pH 7.4
with 1 mM MgCl2 at 4° C. Membranes are collected onto Wallac Filtermat A filters that have been presoaked (and subsequently air-dried) in wash buffer.
Filtermats are air-dried overnight, then placed in bags with 10 ml of Betaplate Scintillant, and counted for 60 sec per sample.

The compounds of Formula I that are basic in nature are capable of forming a wide variety of different salts with various inorganic and organic acids. Although such salts must be pharmaceutically acceptable for administration to animals, it is often desirable in practice to initially isolate a compound of Formula I or from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained.

Those compounds of Formula I that are also acidic in nature, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of Formula I. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they can also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

In the schemes and examples below, the following terms are intended to have the following, general meaning:

BOP=benzotriazol-lyloxy tris(dimethylamino)phosphonium hexafluorophosphate

DMF: dimethyformamide

° C.: degrees Celsius

d; doublet (spectral)

DCE: 1,2-dichlorethane

DMF: dimethyl formamide

DME: dimethoxy methane

GC: gas chromatography

mg: milligrams

HBTU: 0-benzotriazol-1-yl-N,N,N,N-tetramethyluranium hexafluorophospate

Hz: hertz

J: coupling constant (in NMR)

L: liter(s)

LAH: lithium aluminum hydride

MHz: megahertz

m/e mass to charge ratio (in mass spectrometry)

NMR: nuclear magnetic resonance

rt or RT: room temperature

s: singlet (NMR),

t: triplet (NMR)

TEA: triethylacetic acid

TFA: trifluoroacetic acid

THF: tetrahydrofuran

General Synthetic Schemes

The following schemes are representative of methods useful in synthesizing the compound of the present invention they are not to constrain the scope of same in any way. 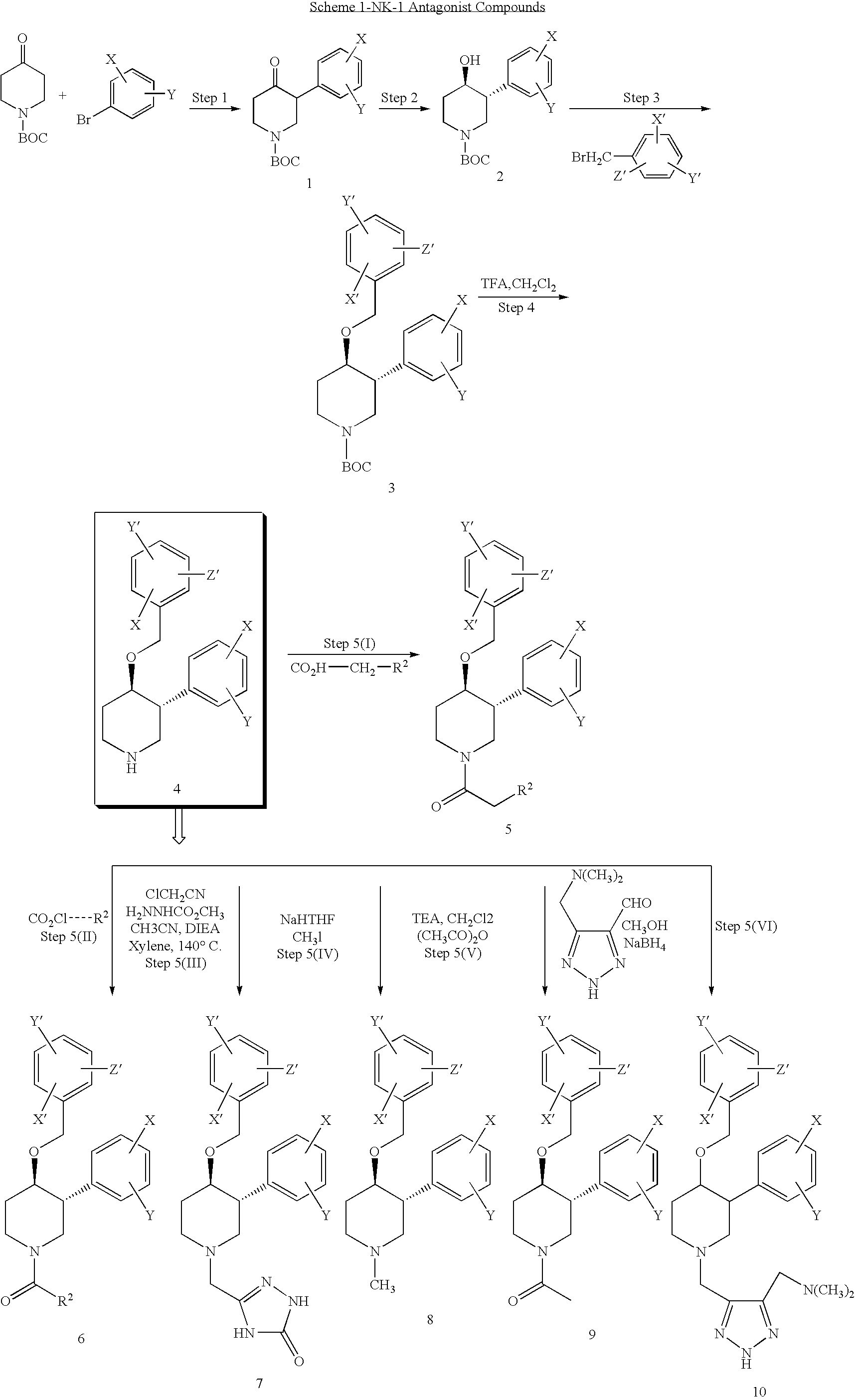
wherein R ², X, Y, X′, Y′, and Z′ are as defined hereinabove.

### Step 1

A reaction container was purged with nitrogen; one or more anhydrous solvents such as ether, dioxan, ethyleneglycoldimethyl ether, THF and DMF, toluene, xylene, and the like, or mixtures thereof; one or more pd-catalysts such as palladium acetate, Pd(PPh₃)₄, Pd₂(dba)₃, Pd(dppf)Cl₂, and the like, or mixtures thereof; and one or more bases such as sodium tert-butoxide, CS₂CO₃, CsF, K₃PO₄, KF, Na₂CO₃, and the like, or mixtures thereof. The mixture is stirred until all of the base dissolves. To the reaction mixture is added a substituted bromobenzene; one or more phosphine catalysts such as tri-tert butylphosphine, Pcy₃, cY₂PCH₂CH₂Pcy₂, dppe, BINAP, PPh₃., and the like, or mixtures thereof; and 1-tert-butoxycarbonyl-4-piperidone. The reaction is slowly heated and then poured into a solution of base such as sodium bicarbonate in water. The resultant intermediary product can be purified according to known methods to give compound (1).

### Step 2

Compound (1) from step 1 is dissolved in one or more solvents such as water, CH₃OH or EtOH, and the like, or mixtures thereof, and bought to about 0° C. One or more borohydrides such as sodium borohydrid, sodium cyanoborohydride, sodium triacetoxy borohydride, and the like, or mixtures thereof, are added and stirred in the reaction mixture. The reaction mixture is then quenched with saturated citric acid, acetic acid or hydrochloric acid. The cis alcohol and trans alcohol intermediary products are then separated and purified according to known methods. The N-BOC-3-R trans alcohol compound (2) is separated according to known methods.

### Step 3

Compound (2) from step 2 is dissolved in one or more solvents such as THF, DMF, DME, and the like, or mixtures thereof, under nitrogen. One or more bases are added such as sodium tert-butoxide, NaH, K₂CO₃, and the like, or mixtures thereof. A substituted or unsubstituted benzylbromide is added and the resulting mixture is refluxed under a nitrogen atmosphere. The resultant intermediate compound (3) is isolated and purified according to known methods.

### Step 4

A solution of compound (3) from step 3 is dissolved in a solution such as CH₂Cl₂/TFA, CH₃OH/HCl, 4M dioxan/HCl, or 2M Ether/HCl and put under nitrogen. The reaction mixture is then poured into saturated NaHCO₃ solution. The resultant intermediate compound (4) was isolated and purified according to known methods.

### Step 5(I)

A solution of compound (4) from step 4 is dissolved in one or more solvents such as CH₂Cl₂, THF, DMF, and the like, or mixtures thereof, under nitrogen. CO₂H—X—CH₂—R, diisopropylethylamine or TEA, and BOP, Py BOP or EDC is added to the reaction mixture, and the reaction mixture is stirred at about room temperature under nitrogen. The reaction mixture is diluted with EtOAc and other solvents such as ether or CH₂Cl₂ and washed and dried according to known methods. The reaction mixture is preferably evaporated to give an oil which is then dissolved in ether, diethylether, or disopropyl ether. A solution of HCl gas in ether, diethylether, or diisopropyl ether is added drop by drop to the ether solution and then the HCl salt is dried under nitrogen to give the compound (5).

### Step 5(II)

A solution compound (4) is dissolved in a suitable solvent such as CH₂Cl₂, DMF, THF, and the like, or mixtures thereof, under nitrogen. A carbonyl chloride compound CO₂Cl—R, diisopropylethylamine (DIPEA) or TEA, and BOP, PyBOP, or EDC are added. The reaction mixture is stirred at room temperature under nitrogen. The reaction mixture is then diluted with EtOAc, Ether, or CH₂Cl₂ and washed with water. The organic layer is dried with MgSO₄ and evaporated to give an oil. This oil is dissolved in ether, diethylether, or diisopropyl ether, and a solution of HCl gas in ether, diethylether, or diisopropyl ether is slowly added. The HCl salt is dried under nitrogen to give compound (6).

### Step 5(III)

A solution of compound (4) is dissolved in a solvent such as CH₃CN, THF, toluene, and the like, or mixtures thereof, under nitrogen. N-methoxycarbonyl-2-chloroacetamidrazone, and diisopropylethylamine or TEA are added, and the reaction mixture is stirred at about room temperature under nitrogen. The reaction mixture is diluted with EtOAc, Ether, or CH₂Cl₂ and washed with water. The organic layer is dried and evaporated according to known methods to give an oil. The oil is dissolved in a high boiling inert solvents, such as xylene, and refluxed under a nitrogen atmosphere. The reaction mixture is then cooled to about room temperature and the solvent was evaporated to give an oil. This oil is combined in ether, diethyl ether or disopropyl ether, and a solution of HCl gas in ether, diethylether, or diisopropyl ether is added slowly. The HCl salt is dried under nitrogen to give compound (7).

### Step 5(IV)

A solution of compound (4) is dissolved in a solvent such as THF or DMF under nitrogen and a methyl halide and a base such as NaH, tBuONa, K₂CO₃, or NaHCO₃ are added. The reaction mixture is stirred under nitrogen at about room temperature. The reaction mixture is then diluted with EtOAc, CH₂Cl₂ or ether and washed with water. The organic layer is dried and evaporated according to known methods to give an oil. This oil is dissolved in ether, diethylether or diisopropylether, and a solution of HCl gas in ether, diethylether, or diisopropyl ether is slowly added. The HCl salt is dried under nitrogen to give compound (8).

### Step 5(V)

A solution of compound (4) is dissolved in a solvent such as CH₂Cl₂, DMF, THF, TEA, and the like, or mixtures thereof. Acetic anhydride is added, and the reaction mixture stirred at room temperature under nitrogen. The reaction mixture is then diluted with EtOAc, CH₂Cl₂ or ether and washed with water. The organic layer is dried and evaporated according to known methods to give an oil. This oil is dissolved in ether, diethylether, or diisopropyl ether and a solution of HCl gas in ether, diethylether, or diisopropyl ether is slowly added. The HCl salt is dried under nitrogen to give compound (9).

### Step 5(VI)

A solution of compound (4) is dissolved in a solvent such as DME or CH₂Cl₂ under nitrogen and 5-dimethylaminomethyl-2H-[1,2,3] triazole-4-carbaldehyde is added. The reaction mixture is stirred at about room temperature under nitrogen. The solvent is then evaporated and the residue is dissolved in solvent such as CH₃OH or EtOH. NaBH₄ or NaBH₃CN is then added and the reaction mixture is stirred at about room temperature under nitrogen. The reaction is quenched with saturated citric acid solution acetic acid, or hydrochloric acid. The solvents are removed and the intermediary product is dried by known methods to afford an oil. This oil is dissolved in ether, diethylether, or diisopropyl ether and a solution of HCl gas in ether, diethylether, or diisopropyl ether is slowly added. The HCl salt is dried under nitrogen to give compound (10). 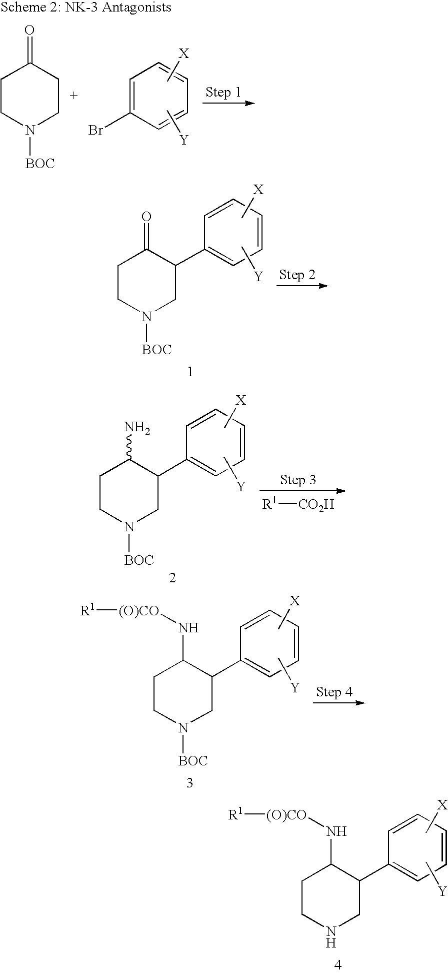
wherein R¹, X and Y are as defined hereinabove.

### Step 1

A reaction container is purged with nitrogen and anhydrous solvents are added such as ether, dioxan, ethylenegycoldimethyl ether, THF and DMF, toluene or xylene, and the like, or mixtures thereof. Palladium acetate or other pd-catalysts such as Pd (PPh₃)₄, Pd₂ (dba)₃, Pd(dppf)Cl₂, and the like, or mixtures thereof, are used. Sodium tert-butoxide or other bases such as Cs₂CO₃, CsF, K₃PO₄, KF, Na₂CO₃, and the like, or mixtures thereof are added to the reaction mixtures and the mixture is stirred until all of the base is dissolved. Phosphine catalysts such as tri-tert butylphosphine, Pcy₃, cy₂PCH₂CH₂Pcy₂, dppe, BINAP, PPh₃, and the like are optionally added to the reaction mixture. A benzyl bromide and 1-tert-butoxycarbonyl-4-piperidone and the reaction is slowly heated at 45-50° C. over a period of about 4 hr to over night to yield compound (1).

### Step 2

A reaction container is purged with nitrogen and 1-tert-butoxycarbonyl-3-(2-methyl-4-fluoro-phenyl)-4-piperidone is dissolved in a solvent such as methanol, ethanol, THF, and the like, or mixtures thereof. Anhydrous ammonium acetate, 4A molecular sieves are added, and the mixture is stirred for about one hour. Sodium cyanoborohydride, sodium borohydride or sodium triacetoxyoborohydride, and the like, or mixtures thereof, are added, and the reaction is stirred at room temperature for about one hour to yield compound (2). The racemic amines are purified preferably by silica gel column.

### Step 3

Compound 2 from step 2 is dissolved in a solvent such as DMF, CH₂Cl₂, and THF, DME, and the like, or mixtures thereof. R¹—COOH is added with diisopropylethylamine, TEA, BOP, PyBOP, DCE, HBTU, and the like, or mixtures thereof. The reaction is stirred over night at room under a nitrogen atmosphere to yield compound (3).

### Step 4

A solution of compound (3) from step 3 is treated with CH₂Cl₂/TFA, CH₃OH/HCl, 4M dioxan/HCl, 2M ether/HCl, and the like, or mixtures thereof, overnight under nitrogen at about room temperature to yield compound (4).

The following examples are illustrative only; they are not restrictive.

### EXAMPLES

### NK-1 Antagonist Compounds

### Intermediate 1

1-tert-butoxycarbonyl-3-(2-methyl-phenyl)-4-piperidone:

A 1-L, three-neck, round bottom flask equipped with a magnetic stirrer and thermometer was purged with nitrogen and charged with anhydrous THF (500 mL), palladium acetate (6.56 g 0.029 mol) and sodium tert-butoxide (42.14 g, 0.44 mol). The mixture was stirred for 15 min until all of the sodium tert-butoxide dissolved. Tri-tert butylphosphine (5.9 g 0.029 mol), 2-methyl-bromobenzene (35.16 mL, 0.29 mol) and 1-tert-butoxycarbonyl-4-piperidone (64.07 g, 0.32 mol) were added, and the reaction was slowly heated at 45-50° C. over a period of 4 hr. The reaction mixture was poured into a solution of sodium bicarbonate (25.0 g) in water (1 L) and extracted with EtOAc (1.0 L). After the organic layer was separated and dried over sodium sulfate, it was concentrated under reduced pressure on a rotary evaporator to dryness to afford 50.0 g of oil. This oil was purified on silica gel flash column, eluting with 15% EtOAc-85% hexane gave 44.64 g (52.77%) of 1-tert-butoxycarbonyl-3-(2-methyl-phenyl)-4-piperidone, as an oil, which solidified standing at room temperature. Mp 97-99° C.; GC/MS m/e 289 (M+), RT=4.87 minutes; 1H-NMR (CDCl₃); δ 1.3(s, 9H), 2.2(s, 3H), 2.6(m, 2H), 3.6(m, 2H), 3.8(m, 1H), 4.3(m, 2H), 7.20 (m 4H).

### Intermediate 2

1-tert-butoxycarbonyl-3-phenyl-4-piperidone:

Using bromobenzene, and following the same procedure as used for intermediate 1 gave 1-tert-Butoxycarbonyl-3-phenyl-4-piperidone. GC/MS m/e 275 (M+)

### Intermediate 3

1-tert-butoxycarbonyl-3-(4-fluoro-phenyl)-4-piperidone:

Using 4-fluorobromobenzene, and following the same procedure as used for intermediate 1 gave 1-tert-butoxycarbonyl-3-(4-fluoro-phenyl)-4-piperidone. GC/MS m/e 293 (M+)

### Intermediate 4

1-tert-butoxycarbonyl-3-(2-methyl-4-fluoro-phenyl)-4-piperidone:

Using 4-fluoro-2-methylbromobenzene, and following the same procedure as used for intermediate 1 gave 1-tert-butoxycarbonyl-3-(2-methyl-4-fluoro-phenyl)-4-piperidone. GC/MS m/e 307 (M+)

### Intermediate 5

1-tert-butoxycarbonyl-3-(3,4-difluoro-phenyl)-4-piperidone:

Using 3,4-fluorobromobenzene, and following the same procedure as used for intermediate 1 gave 1-tert-butoxycarbonyl-3-(2-methyl-4-fluoro-phenyl)-4-piperidone. GC/MS m/e 311 (M+)

### Intermediate 6

1-tert-butoxycarbonyl-3-(2-pyridyl)-4-piperidone:

Using 2-bromopyridine, and following the same procedure as used for intermediate 1 gave 1-tert-butoxycarbonyl-3-(2-pyridyl)-4-piperidone. Fab/MS m/e 277 (M+1)

### Intermediate 7

1-tert-butoxycarbonyl-3-(3-chloro-phenyl)-4-piperidone:

Using 3-chloro-3-bromobenzene, and following the same procedure as used for intermediate 1 gave 1-tert-butoxycarbonyl-3-(3-chloro-phenyl)-4-piperidone. Fab/MS m/e 295 (M+2)

### Intermediate 8

1-tert-butoxycarbonyl-3R-(2-methyl-phenyl)-4S-hydroxypiperidine:

1-tert-butoxycarbonyl-3-(2-methyl-phenyl)-4-piperidone (Intermediate 1) (8.0 g 0.027 mol) was dissolved in CH₃OH (250 mL) and bought to 0° C. using ice water. Sodium borohydride (1.0 g 0.027 mol) was added and the reaction mixture was stirred for thirty minutes. The reaction was then quenched with saturated citric acid solution. The methanol was removed by rotary evaporation. The water layer was made basic and extracted with EtOAc (400 mL). The combined extracts were washed with water, dried over sodium sulfate, and concentrated under reduced pressure on a rotary evaporator to dryness to afford 8.0 g of racemic alcohols as an an oily product. The racemic alcohols were purified by silica gel column, eluting with 20% EtOAc-80% hexane to give 3.38 g of cis alcohol (Rf=0.4 in 30% EtOAc/Hexane) and 4.58 g of trans alcohol (Rf=0.3 in 30% EtOAc/Hexane). The trans alcohol was separated by chiral column gave 2.3 g of N-BOC-3R-(2-methyl-phenyl)-4S-hydroxypiperidine. GC/MS m/e 291 (M+), RT=4.92 minutes; 1H-NMR (CDCl3); δ 1.4(s 9H); 1.6(m, 2H); 2.4 (s, 3H); 2.6 (t, J=7 Hz, 1H); 2.8 (m, 2H); 4.0 (m, 2H); 4.2 (b-m, OH); 7.20 (m, 4H).

### Intermediate 9

1-tert-butoxycarbonyl-3-phenyl-4-hydroxypiperidine:

Using 1-tert-butoxycarbonyl-3-(phenyl)-4-piperidone (Intermediate 2), and following the same procedure used for preparing Intermediate 8 gave 1-tert-butoxycarbonyl-3-phenyl-4-hydroxypiperidine. GC/MS m/e 277 (M+)

### Intermediate 10

1-tert-butoxycarbonyl-3-(4-fluoro-phenyl)-4-hydroxypiperidine:

Using 1-tert-butoxycarbonyl-3-(4-fluorophenyl)-4-piperidone (Intermediate 3), and following the same procedure used for preparing Intermediate 8 gave 1-tert-butoxycarbonyl-3-(4-fluoro-phenyl)-4-hydroxypiperidine. GC/MS m/e 295 (M+)

### Intermediate 11

1-tert-butoxycarbonyl-3-(2-methyl-4-fluoro-phenyl)-4-hydroxypiperidine:

Using 1-tert-butoxycarbonyl-3(4-fluoro-2-methylphenyl)-4-piperidone (Intermediate 4), and following the same procedure as used for intermediate 8 gave 1-tert-butoxycarbonyl-3-(2-methyl-4-fluoro-phenyl)-4-hydroxypiperidine. GC/MS m/e 309 (M+)

### Intermediate 12

1-tert-butoxycarbonyl-3-(3,4-difluoro-phenyl)-4-hydroxypiperidine:

Using 1-tert-butoxycarbonyl-3-(3,4-difluorophenyl)-4-piperidone (Intermediate 5), and following the same procedure as used for Intermediate 8 gave 1-tert-butoxycarbonyl-3-(3,4-difluoro-phenyl)-4-hydroxypiperidine. Fab/MS m/e 214 (M-BOC

### Intermediate 13

1-tert-butoxycarbonyl-3-(2-pyridyl)-4-hydroxypiperidine:

Using 1-tert-butoxycarbonyl-3-(2-pyridyl)-4-piperidone (Intermediate 6), and following the same procedure as used for intermediate 8 gave 1-tert-butoxycarbonyl-3-(2-pyridyl)-4-hydroxypiperidine. Fab/MS m/e 279 (M+1)

### Intermediate 14

1-tert-butoxycarbonyl-3-(3-chloro-phenyl)-4-hydroxypiperidine:

Using 1-tert-butoxycarbonyl-3-(3-chlorobromophenyl)-4-piperidonen (Intermediate 7), and following the same procedure as used for intermediate 8 gave 1-tert-butoxycarbonyl-3-(3-chloro-phenyl)-4-hydroxypiperidine. Fab/MS m/e 312 (M+1)

### Intermediate 15

Preparation of 1-tert-Butoxycarbonyl-4S-(3,5-Bis-trifluoromethyl-benzyloxy)-3R-2-tolyl-piperidine:

A solution of 586 mg (2.011 mmol) of trans alcohol (1-tert-butoxycarbonyl-3R-(2-methyl-phenyl)-4S-hydroxypiperidine) (Intermediate 8) in 20 mL of THF under nitrogen and 290 mg (3.016 mmol) of sodium tert-butoxide were added together. A suspension resulted and after 15 minutes, 0.60 mL (3.016 mmol) of 3,5-bis (trifluoromethyl) benzylbromide were added and the resulting mixture was refluxed gently for three hours under a nitrogen atmosphere. The reaction mixture was cooled to room temperature and then poured into ice water. This was extracted three times with EtOAc. The combined EtOAc extracts were washed with saturated NaCl solution and dried with MgSO₄. Evaporation of the solvent gave 650 mg of yellow oil. This oil was purified on silica gel flash column, eluting with 15% EtOAc-85% hexane gave 617 mg (60%), as oil. Fab/MS m/e 418 (M-BOC); 1H-NMR (CDCl₃); δ1.2 (S, 9H); 2.6 (m, 1H); 2.2 (d, J=7 Hz, 1H), 2.4(s, 3H), 2.8(m, 2H), 3.0(m, 1H), 3.7(m, 1H), 4.0(m, 1H), 4.2(d, J=12 Hz, 1H), 4.26(b-m, 1H), 4.6(d, J=12 Hz, 1H), 7.08(m, 4H), 7.40(S, H), 6.9(s, 1H).

### Intermediate 16

Preparation of 1-tert-Butoxycarbonyl-4-(3,5-Bis-trifluoromethyl-benzyloxy)-3-phenyl-piperidine:

Using 1-tert-Butoxycarbonyl-3R-(phenyl)-4S-hydroxypiperidine (Intermediate 9), and following the same procedure as used for intermediate 15 gave 1-tert-butoxycarbonyl-4-(3,5-bis-trifluoromethyl-benzyloxy)-3-phenyl-piperidine. Fab/MS m/e 404 (M-BOC)

### Intermediate 17

1-tert-butoxycarbonyl-4-(3,5-bis-trifluoromethyl-benzyloxy)-3-(4-fluoro-phenyl)-piperidine:

Using 1-tert-Butoxycarbonyl-3R-(4-fluorophenyl)-4S-hydroxypiperidine (Intermediate 10), and following the same procedure as used for intermediate 15 gave 1-tert-butoxycarbonyl-4-(3,5-bis-trifluoromethyl-benzyloxy)-3-(4-fluoro-phenyl)-piperidine. Fab/MS m/e 422 (M-BOC)

### Intermediate 18

1-tert-butoxycarbonyl-4-(3,5-bis-trifluoromethyl-benzyloxy)-3-(2-methyl-4-fluoro-phenyl)-piperidine:

Using 1-tert-butoxycarbonyl-3R-(2-methyl-4-fluoro-phenyl)-4S-hydroxypiperidine (Intermediate 11), and following the same procedure as used for intermediate 15 gave 1-tert-butoxycarbonyl-4-(3,5-bis-trifluoromethyl-benzyloxy)-3-(2-methyl-4-fluoro-phenyl)-piperidine. Fab/MS m/e 436 (M-BOC)

### Intermediate 19

1-tert-Butoxycarbonyl-4-(3,5-Bis-trifluoromethyl-benzyloxy)-3-(3,4-difluoro-phenyl)-piperidine:

Using 1-tert-Butoxycarbonyl-3R-(3,4-difluorophenyl)-4S-hydroxypiperidine (Intermediate 14), and following the same procedure as used for intermediate 16 gave 1-tert-Butoxycarbonyl-4-(3,5-Bis-trifluoromethyl-benzyloxy)-3-(3,4-difluoro-phenyl)-piperidine. Fab/MS m/e 540 (M+1)

### Intermediate 20

Preparation of 1-tert-butoxycarbonyl-4-(3,5-Bis-trifluoromethyl-benzyloxy)-3-(2-pyridyl)-piperidine (3-5):

Using 1-tert-butoxycarbonyl-3R-(2-pyridyl)-4S-hydroxypiperidine (Intermediate 12), and following the same procedure as used for intermediate 16 gave 1-tert-butoxycarbonyl-4-(3,5-Bis-trifluoromethyl-benzyloxy)-3-(2-pyridyl)-piperidine. Fab/MS m/e 505 (M-BOC)

### Intermediate 21

Preparation of 1-tert-Butoxycarbonyl-4-(3,5-Bis-trifluoromethyl-benzyloxy)-3-(3-chloro-phenyl)-piperidine (3-6):

Using 1-tert-Butoxycarbonyl-3R-(3-chlorophenyl)-4S-hydroxypiperidine (Intermediate 13), and following the same procedure as used for intermediate 15 gave 1-tert-butoxycarbonyl-4-(3,5-Bis-trifluoromethyl-benzyloxy)-3-(3-chloro-phenyl)-piperidine. Fab/MS m/e 538 (M+1)

### Intermediate 22

Pyrrolidin-1-yl-acetic Acid Methyl Ester:

Pyrrolidine 10.0 g (0.140 mol) was dissolved in CH₂Cl₂ and methylbromoacetate 22.0 g (0.143 mol), KOH 8.24 g (0.146 mol), and K₂CO₃ (0.144 mol) were added. The reaction was stirred at room temperature for about three hours. The reaction mixture was then diluted with CH₂Cl₂ (100 ml) and washed with water three times. The organic layer was dried with MgSO₄. Evaporation of most of the solvent gave 13 g of pyrrolidin-1-yl-acetic acid methyl ester as an oil. GC/MS m/e 143 (M+); ¹H NMR (CDCl₃) δ3.2 (σ, 2H), 3.6(s, 3H, OCH₃).

### Intermediate 23

Pyrrolidin-1-yl-acetic Acid:

Intermediate 23 13.30 g (93.0 mmol) was combined with 200 ml of water and the reaction mixture was refluxed overnight. Water was concentrated under reduced pressure on a rotary evaporator to dryness and treated with benzene or toluene to evaporate to dryness affording 11.80 g of pyrrolidin-1-yl-acetic acid as an oil. GC/MS m/e 129 (M+)

### Intermediate 24

Piperidine-1-yl-acetic Acid:

Using piperidine-1-yl-acetic acid methyl ester (purchased from Aldrich Chemical Company or Aztec Chemical Company) and following the same procedure as used for Intermediate 23 gave piperidine-1-yl-acetic acid. GC/MS m/e 144 (M+)

### Intermediate 25

Morpholin-4-yl-acetic Acid Methyl Ester:

Using morpholine and methyl bromoacetate and following the same procedure as used for Intermediate 22 gave morpholin-4-yl-acetic acid methyl ester. GC/MS m/e 159 (M+)

### Intermediate 26

Morpholin-4-yl-acetic Acid:

Using Intermediate 25 and following the same procedure as used for Intermediate 23 gave morpholine-4-yl-acetic acid. GC/MS m/e 145 (M+)

### Intermediate 27

4-methoxycarbonylmethyl-piperazine-1-carboxylic Acid tert-butyl Ester:

Using piperazine-1-carboxylic acid tert-butyl ester and methyl bromoacetate and following the same procedure as used for Intermediate 22 gave 4-methoxycarbonylmethyl-piperazine-1-carboxylic acid tert-butyl ester. GC/MS m/e 258 (M+)

### Intermediate 28

4-carboxymethyl-methyl-piperazine-1-carboxylic Acid tert-butyl Ester:

Using Intermediate 27 and following the same procedure as used for Intermediate 22 gave 4-carboxymethyl-methyl-piperazine-1-carboxylic acid tert-butyl ester. GC/MS m/e 244 (M+)

### Intermediate 29

(4-methyl-piperazine-1-yl)-acetic Acid Methyl Ester:

Using 4-methyl piperazine and methyl bromoacetate and following the same procedure as used for Intermediate 22 gave (4-methyl-piperazine-1-yl)-acetic acid methyl ester. GC/MS m/e 172 (M+)

### Intermediate 30

(4-methyl-piperazine-1-yl)-acetic Acid:

Using Intermediate 29 and following the same procedure as used for Intermediate 23 gave (4-methyl-piperazine-1-yl)-acetic acid. GC/MS m/e 158 (M+)

### Intermediate 31

(4-ethyl-piperazine-1-yl)-acetic Acid Methyl Ester:

Using 4-methyl piperazine and methyl bromoacetate and following the same procedure as used for Intermediate 22 gave (4-ethyl-piperazine-1-yl)-acetic acid methyl ester. GC/MS m/e 186 (M+)

### Intermediate 32

(4-Ethyl-piperazine-1-yl)-acetic Acid:

Using Intermediate 31 and following the same procedure as used for Intermediate 23 gave (4-Ethyl-piperazine-1-yl)-acetic acid. GC/MS m/e 172 (M+)

### Intermediate 33

(2-oxo-pyrrolidin-1-yl)acetic Acid:

Using (2-oxo-pyrrolidin-1-yl)acetic acid methyl ester (purchased from Aldrich Chemical Company or Aztec Chemical Company) and following the same procedure as used for Intermediate 23 gave (2-oxo-pyrrolidin-1-yl)acetic acid. GC/MS m/e 144 (M+1)

### Intermediate 34

(2-oxo-piperidine-1-yl)-acetic Acid Methyl Ester:

Using 2-oxo-piperidine and methyl bromoacetate and following the same procedure as used for Intermediate 22 gave (2-oxo-piperidine-1-yl)-acetic acid methyl ester. GC/MS m/e 99 (M—CH₂CO₂CH₃)

### Intermediate 35

(2-oxo-piperidine-1-yl)-acetic Acid:

Using Intermediate 34 and following the same procedure as used for Intermediate 23 gave (2-oxo-piperidine-1-yl)-acetic acid.

APCI m/e 156 (M−1)

### Intermediate 36

(4-Benzyl-piperazine-1-yl)-acetic Acid:

Using (4-benzyl-piperazine-1-yl)-acetic acid methyl ester and following the same procedure as used for Intermediate 23 gave (4-benzyl-piperazine-1-yl)-acetic acid. C/MS m/e 234 (M+)

### Intermediate 37

(4-Acetyl-piperazine-1-yl)-acetic Acid Methyl Ester:

Using 4-Acetyl-piperazine and methyl bromoacetate and following the same procedure as used for Intermediate 22 gave (4-Acetyl-piperazine-1-yl)-acetic acid methyl ester. GC/MS m/e 214 (M+)

### Intermediate 38

(4-Acetyl-piperazine-1-yl)-acetic Acid:

Using Intermediate 37 and following the same procedure as used for Intermediate 23 gave (4-acetyl-piperazine-1-yl)-acetic acid. GC/MS m/e 186 (M+)

### Example 1

### 4S-(3,5-bis-trifluoromethyl-benzloxy)-3R-2-tolyl-piperidine

A solution of 617 mg (1.192 mmol) of 1-tert-butoxycarbonyl-4S-(3,5-bis-trifluoromethyl-benzloxy)-3R-(2-tolyl)-piperidine (Intermediate 15) was prepared in 20 mL of CH₂Cl₂ and then 1.0 mL of TFA. The reaction mixture was stirred overnight under nitrogen atmosphere. The reaction mixture was poured into saturated NaHCO₃ solution and extracted three times with EtOAc. The combined EtOAc extracts were washed with saturated NaCl solution and dried with MgSO₄. Evaporation of most solvent gave 540 mg of oil. Fab/MS m/e 418 (M+1); 1H NMR (CD₃Od); δ1.8(m, 1H), 2.30(s, 3H), 2.6(d, J=7 Hz, 1H), 3.2 (m, 2H), 3.4(m, 2H), 3.6(d, J=7 Hz, 1H), 3.98(m, 1H), 4.22(d, J=12 Hz 1H), 4.7(d, J=12 Hz, 1H), 7.1 (m, 3H), 7.3(d, J=7 Hz, 1H), 7.42(s, 2H), 6.98(s, 1H).

### Example 2

### 4-(3,5-bis-trifluoromethyl-benzloxy)-3-phenyl-piperidine

Using 1-tert-Butoxycarbonyl-4S-(3,5-bis-trifluoromethyl-benzloxy)-3R-phenyl-piperidine (Intermediate 16) and following the same procedure as used for Example 1 gave 4-(3,5-bis-trifluoromethyl-benzloxy)-3-phenyl-piperidine. Fab/MS m/e 404 (M+1)

### Example 3

### 4-(3,5-bis-trifluoromethyl-benzyloxy)-3-(4-fluoro-phenyl)-piperidine

Using 1-tert-Butoxycarbonyl-4S-(3,5-bis-trifluoromethyl-benzloxy)-3R-(4-fluorophenyl)-piperidine (Intermediate 17) and following the same procedure as used for Example 1 gave 4-(3,5-bis-trifluoromethyl-benzyloxy)-3-(4-fluoro-phenyl)-piperidine. Fab/MS m/e 422 (M+1)

### Example 4

### 4-(3,5-bis-trifluoromethyl-benzyloxy)-3-(2-methyl-4-fluoro-phenyl)-piperidine

Using 1-tert-Butoxycarbonyl-4S-(3,5-bis-trifluoromethyl-benzyloxy)-3R-(2-methyl-4-fluorophenyl)-piperidine (Intermediate 18) and following the same procedure as used for Example 1 gave 4-(3,5-bis-trifluoromethyl-benzyloxy)-3-(2-methyl-4-fluoro-phenyl)-piperidine. Fab/MS m/e 436 (M+1)

### Example 5

### 4-(3,5-bis-trifluoromethyl-benzyloxy)-3-(3,4-difluoro-phenyl)-piperidine

Using 1-tert-Butoxycarbonyl-4S-(3,5-bis-trifluoromethyl-benzloxy)-3R-(3,4-difluorophenyl)-piperidine (Intermediate 19) and following the same procedure as used for Example 1 gave 4-(3,5-bis-trifluoromethyl-benzyloxy)-3-(3,4-difluoro-phenyl)-piperidine. Fab/MS m/e 400 (M+1)

### Example 6

### 4-(3,5-bis-trifluoromethyl-benzyloxy)-3-(2-pyridyl)-piperidine

Using 1-tert-Butoxycarbonyl-4S-(3,5-bis-trifluoromethyl-benzloxy)-3R-(2-pyridyl)-piperidine (intermediate 20) and following the same procedure as used for Example 1 gave 4-(3,5-bis-trifluoromethyl-benzyloxy)-3-(2-pyridyl)-piperidine. Fab/MS m/e 405 (M+1)

### Example 7

### 4-(3,5-bis-trifluoromethyl-benzyloxy)-3-(3-chloro-phenyl)-piperidine

Using 1-tert-Butoxycarbonyl-4S-(3,5-bis-trifluoromethyl-benzloxy)-3R-(3-chlorophenyl)-piperidine (Intermediate 21) and following the same procedure as used for Example 1 gave 4-(3,5-bis-trifluoromethyl-benzyloxy)-3-(3-chloro-phenyl)-piperidine. Fab/MS m/e 438 (M+1)

### Example 9

### 4-(4-bromo-benzyloxy)-3-(2-methyl-4-fluoro-phenyl)-piperidine

4-Bromobenzyl bromide (commercially available) was placed in 2 dram vials (0.1125 mmol, 1.5 eq.). (1-tert-butoxycarbonyl-3R-(2-methyl-4-fluorophenyl)-4S-hydroxypiperidine (Intermediate 11) 1.0 eq., 0.075 mmol in 0.888 ml THF) and KOt-Butoxide (1.0 M in THF, 1.5 eq. 0.113 ml) were added to the 4-bromobenzyl bromide. The reaction mixture was agitated and heated for about 8 hrs at 80° C. in sealed vials. 2 ml water and 2.4 ml EtOAc were added, and the mixture was agitated. The organic layer was removed and passed through Na₂SO₄ in a SPE cartridge into a tared 2 dram vial. The extraction was repeated twice and the samples were dried down.

To the reaction mixtures, 1 ml of a 1:1 of TFA:CH₂Cl₂ was added and the reaction mixture was agitated overnight in sealed vials. 2 ml of 2 N NaOH and 2.4 ml CH₂Cl₂ were then added. The organics were separated and loaded onto a conditioned SCX SPE (1 g, 6 ml). Extraction was repeated twice. The reaction mixtures were eluted with a 1:1 ratio of MeOH/CH₂Cl₂. The reaction mixtures were then eluted with 1 N TEA in MeOH. (MS esi m/z 379, 380 (M+1 and M+2)

### Example 10

### 4-(3-bromo-benzyloxy)-3-(2-methyl-4-fluoro-phenyl)-piperidine

Using Intermediate 11 and 3-Bromobenzyl bromide, and following the same procedure as used in Example 9 gave 4-(3-bromo-benzyloxy)-3-(2-methyl-4-fluoro-phenyl)-piperidine. (MS esi m/z 379, 380 (M+1 and M+2)

### Example 11

### 4-(2-bromo-benzyloxy)-3-(2-methyl-4-fluoro-phenyl)-piperidine

Using Intermediate 11 and 2-Bromobenzyl bromide, and following the same procedure as used in Example 9 gave 4-(2-bromo-benzyloxy)-3-(2-methyl-4-fluoro-phenyl)-piperidine. MS esi m/z 379, 380 (M+1 and M+2)

### Example 12

### 4-(2,6-dichloro-benzyloxy)-3-(2-methyl-4-fluoro-phenyl)-piperidine

Using Intermediate 11 and 2,6-dichloro-benzyl bromide, and following the same procedure as used in Example 9 gave 4-(2,6-dichloro-benzyloxy)-3-(2-methyl-4-fluoro-phenyl)-piperidine. MS esi m/z 369 (M+1)

### Example 13

### 4-(2,5-dichloro-benzyloxy)-3-(2-methyl-4-fluoro-phenyl)-piperidine

Using Intermediate 11 and 2,5-dichloro-benzyl bromide, and following the same procedure as used in Example 9 gave 4-(2,5-dichloro-benzyloxy)-3-(2-methyl-4-fluoro-phenyl)-piperidine. MS esi m/z 369 (M+1)

### Example 14

### 4-(3,5-dichloro-benzyloxy)-3-(2-methyl-4-fluoro-phenyl)-piperidine

Using Intermediate 11 and 3,5-dichloro-benzyl bromide, and following the same procedure as used in Example 9 gave 4-(3,5-dichloro-benzyloxy)-3-(2-methyl-4-fluoro-phenyl)-piperidine. MS esi m/z 369 (M+1)

### Example 15

### 4-(4-fluoro-benzyloxy)-3-(2-methyl-4-fluoro-phenyl)-piperidine

Using Intermediate 11 and 4-fluoro-benzyl bromide, and following the same procedure as used in Example 9 gave 4-(4-fluoro-benzyloxy)-3-(2-methyl-4-fluoro-phenyl)-piperidine. MS esi m/z 318 (M+1)

### Example 16

### 4-(2-fluoro-benzyloxy)-3-(2-methyl-4-fluoro-phenyl)-piperidine

Using Intermediate 11 and 2-fluoro-benzyl bromide, and following the same procedure as used in Example 9 gave 4-(2-fluoro-benzyloxy)-3-(2-methyl-4-fluoro-phenyl)-piperidine. MS esi m/z 318 (M+1)

### Example 17

### 4-(3-fluoro-benzyloxy)-3-(2-methyl-4-fluoro-phenyl)-piperidine

Using Intermediate 11 and 3-fluoro-benzyl bromide, and following the same procedure as used in Example 9 gave 4-(3-fluoro-benzyloxy)-3-(2-methyl-4-fluoro-phenyl)-piperidine. MS esi m/z 318 (M+1)

### Example 18

### 4-(3,4-difluoro-benzyloxy)-3-(2-methyl-4-fluoro-phenyl)-piperidine

Using Intermediate 11 and 3,4-Difluoro-benzyl bromide, and following the same procedure as used in Example 9 gave 4-(3,4-difluoro-benzyloxy)-3-(2-methyl-4-fluoro-phenyl)-piperidine. MS esi m/z 336 (M+1)

### Example 19

### 4-(3,5-difluoro-benzyloxy)-3-(2-methyl-4-fluoro-phenyl)-piperidine

Using Intermediate 11 and 3,4-difluoro-benzyl bromide, and following the same procedure as used in Example 9 gave 4-(3,4-difluoro-benzyloxy)-3-(2-methyl-4-fluoro-phenyl)-piperidine. MS esi m/z 336 (M+1)

### Example 20

### 4-(2,6-difluoro-benzyloxy)-3-(2-methyl-4-fluoro-phenyl)-piperidine

Using Intermediate 11 and 2,6-difluoro-benzyl bromide, and following the same procedure as used in Example 9 gave 4-(2,6-di-fluoro-benzyloxy)-3-(2-methyl-4-fluoro-phenyl)-piperidine. MS esi m/z 336 (M+1)

### Example 21

### 4-(3,6-difluoro-benzyloxy)-3-(2-methyl-4-fluoro-phenyl)-piperidine

Using Intermediate 11 and 3,6-difluoro-benzyl bromide, and following the same procedure as used in Example 9 gave 4-(3,6-di-fluoro-benzyloxy)-3-(2-methyl-4-fluoro-phenyl)-piperidine. MS esi m/z 336 (M+1)

### Example 22

### 4-(2,4,6-tri-fluoro-benzyloxy)-3-(2-methyl-4-fluoro-phenyl)-piperidine

Using Intermediate 11 and 2,4,6-trifluoro-benzyl bromide, and following the same procedure as used in Example 9 gave 4-(2,4,6-tri-fluoro-benzyloxy)-3-(2-methyl-4-fluoro-phenyl)-piperidine. MS esi m/z 354 (M+1)

### Example 23

### 4-(2,3,6-trifluoro-benzyloxy)-3-(2-methyl-4-fluoro-phenyl)-piperidine

Using Intermediate 11 and 2,3,6-trifluoro-benzyl bromide, and following the same procedure as used in Example 9 gave 4-(2,3,6-tri-fluoro-benzyloxy)-3-(2-methyl-4-fluoro-phenyl)-piperidine. MS esi m/z 354 (M+1)

### Example 24

### 4-(4-trifluoromethyl-benzyloxy)-3-(2-methyl-4-fluoro-phenyl)-piperidine

Using Intermediate 11 and 4-trifluoromethyl-benzyl bromide, and following the same procedure as used in Example 9 gave 4-(4-tri-fluoromethyl-benzyloxy)-3-(2-methyl-4-fluoro-phenyl)-piperidine. MS esi m/z 368 (M+1)

### Example 25

### 4-(3-trifluoromethyl-benzyloxy)-3-(2-methyl-4-fluoro-phenyl)-piperidine

Using Intermediate 11 and 3-trifluoromethyl-benzyl bromide, and following the same procedure as used in Example 9 gave 4-(3-trifluoromethyl-benzyloxy)-3-(2-methyl-4-fluoro-phenyl)-piperidine. MS esi m/z 368 (M+1)

### Example 26

### 4-(3,5-bis-trifluoromethyl-benzyloxy)-3-(2-methyl-4-fluoro-phenyl)-piperidine

Using Intermediate 11 and 3,5-bistrifluoromethyl-benzyl bromide, and following the same procedure as used in Example 9 gave 4-(3,5-bis-trifluoromethyl-benzyloxy)-3-(2-methyl-4-fluoro-phenyl)-piperidine. MS esi m/z 436 (M+1)

### Example 27

### 4-(2,4-bis-trifluoromethyl-benzyloxy)-3-(2-methyl-4-fluoro-phenyl)-piperidine

Using Intermediate 11 and 2,4-bistrifluoromethyl-benzyl bromide, and following the same procedure as used in Example 9 gave 4-(2,4-bis-trifluoromethyl-benzyloxy)-3-(2-methyl-4-fluoro-phenyl)-piperidine. MS esi m/z 436 (M+1)

### Example 28

### 4-(4-trifluoromethoxyl-benzyloxy)-3-(2-methyl-4-fluoro-phenyl)-piperidine

Using Intermediate 11 and 4-trifluoromethoxyl-benzyl bromide, and following the same procedure as used in Example 9 gave 4-(4-trifluoromethoxyl-benzyloxy)-3-(2-methyl-4-fluoro-phenyl)-piperidine. MS esi m/z 384 (M+1)

### Example 29

### 4-(2-methyl-3,4-disfluorobenzyloxy)-3-(2-methyl-4-fluoro-phenyl)-piperidine

Using Intermediate 11 and 2-methyl-3,4-difluorobenzylbromide, and following the same procedure as used in Example 9 gave 4-(2-methyl-3,4-difluorobenzyloxy)-3-(2-methyl-4-fluoro-phenyl)-piperidine. MS esi m/z 364 (M+1)

### Example 30

### 4-(2-methyl-3,5-bis-fluoromethyl-benzyloxy)-3-(2-methyl-4-fluoro-phenyl)-piperidine

Using Intermediate 11 and 2-methyl-3,5-difluorobenzyl bromide, and following the same procedure as used in Example 9 gave 4-(2-methyl-3,5-di-fluoro-benzyloxy)-3-(2-methyl-4-fluoro-phenyl)-piperidine. MS esi m/z 364 (M+1)

### Example 31

### 4-(2-ethyl-3,5-difluoromethyl-benzyloxy)-3-(2-methyl-4-fluoro-phenyl)-piperidine

Using Intermediate 11 and 2-ethyl-3,5-difluorobenzyl bromide, and following the same procedure as used in Example 9 gave 4-(2-ethyl-3,5-difluoromethyl-benzyloxy)-3-(2-methyl-4-fluoro-phenyl)-piperidine. MS esi m/z 378 (M+1)

### Example 32

### 4-(2-methyl-benzyloxy)-3-(2-methyl-4-fluoro-phenyl)-piperidine

Using Intermediate 11 and 2-methyl benzyl bromide, and following the same procedure as used in Example 9 gave 4-(2-methyl-benzyloxy)-3-(2-methyl-4-fluoro-phenyl)-piperidine. MS esi m/z 314 (M+1)

### Example 33

### 4-(2-chloro-5-methoxy-benzyloxy)-3-(2-methyl-4-fluoro-phenyl)-piperidine

Using Intermediate 11 and 2-chloro-5-methoxy-benzyl bromide, and following the same procedure as used in Example 9 gave 4-(2-chloro-5-methoxy-benzyloxy)-3-(2-methyl-4-fluoro-phenyl)-piperidine. MS esi m/z 364 (M+1)

### Example 34

### 4-[3-(2-methyl-4-fluoro-phenyl)-piperidine-4-yloxymethyl]-3-methoxy-benzoic Acid Methylester

Using Intermediate 11 and 3-methoxy-4-bromomethyl-benzoic acid methylester, and following the same procedure as used in Example 9 gave 4-[3-(2-methyl-4-fluoro-phenyl)-piperidine-4-yloxymethyl]-3-methoxy-benzoic acid methylester. MS esi m/z 388 (M+1)

### Example 35

### 4-(3-methoxy-6-bromo-benzyloxy)-3-(2-methyl-4-fluoro-phenyl)-piperidine

Using Intermediate 11 and 3-methoxy-6-bromo-benzyl bromide, and following the same procedure as used in Example 9 gave 4-(3-methoxy-6-bromo-benzyloxy)-3-(2-methyl-4-fluoro-phenyl)-piperidine. MS esi m/z 409 (M+1)

### Example 36

### 4-(3-iodo-4-chloro-benzyloxy)-3-(2-methyl-4-fluoro-phenyl)-piperidine

Using Intermediate 11 and 3-iodo-4-chloro-benzyl bromide, and following the same procedure as used in Example 9 gave 4-(3-iodo-4-chloro-benzyloxy)-3-(2-methyl-4-fluoro-phenyl)-piperidine. MS esi m/z 460 (M+1)

### Example 37

### 4-(Biphenyl-3-ylmethoxy)-3-(2-methyl-4-fluoro-phenyl)-piperidine

Using Intermediate 11 and biphenyl-3-yl-bromomethyl, and following the same procedure as used in Example 9 gave 4-(biphenyl-3-ylmethoxy)-3-(2-methyl-4-fluoro-phenyl)-piperidine. MS esi m/z 376 (M+1)

### Example 38

### 4-[2-(4-Fluoro-benzyl)-benzyloxy]-3-(4-fluoro-2-methyl-phenyl)-piperidine

Using Intermediate 11 and 2-(4-fluoro-benzyl)-benzyl bromide, and following the same procedure as used in Example 9 gave 4-[2-(4-Fluoro-benzyl)-benzyloxy]-3-(4-fluoro-2-methyl-phenyl)-piperidine. MS esi m/z 408 (M+1)

### Example 39

### 4-(3-methyl-benzyloxy)-3-(2-methyl-4-fluoro-phenyl)-piperidine

Using Intermediate 11 and 3-methyl-benzyl bromide, and following the same procedure as used in Example 9 gave 4-(3-methyl-benzyloxy)-3-(2-methyl-4-fluoro-phenyl)-piperidine: (4), MS esi m/z 314 (M+1)

### Example 40

### 4-(2-trifluoromethyl-benzyloxy)-3-(2-methyl-4-fluoro-phenyl)-piperidine

Using Intermediate 11 and 2-trifluoromethyl-benzyl bromide, and following the same procedure as used in Example 9 gave 4-(2-trifluoromethyl-benzyloxy)-3-(2-methyl-4-fluoro-phenyl)-piperidine. MS esi m/z 368 (M+1)

### Example 41

### 4-(3,4-dimethyl-benzyloxy)-3-(2-methyl-4-fluoro-phenyl)-piperidine

Using Intermediate 11 and 3,4-dimethyl-benzyl bromide, and following the same procedure as used in Example 9 gave 4-(3-4-dimethyl-benzyloxy)-3-(2-methyl-4-fluoro-phenyl)-piperidine. MS esi m/z 328 (M+1)

### Example 42

### 4-(3-methyl-5-fluoro-benzyloxy)-3-(2-methyl-4-fluoro-phenyl)-piperidine

Using Intermediate 11 and 3-methyl-5-fluoro-benzyl bromide, and following the same procedure as used in Example 9 gave 4-(3-methyl-5-fluoro-benzyloxy)-3-(2-methyl-4-fluoro-phenyl)-piperidine. MS esi m/z 332 (M+1)

### Example 43

### 4-(2-methyl-3-fluoro-benzyloxy)-3-(2-methyl-4-fluoro-phenyl)-piperidine

Using Intermediate 11 and 2-methyl-3-fluoro-benzyl bromide, and following the same procedure as used in Example 9 gave 4-(2-methyl-3-fluoro-benzyloxy)-3-(2-methyl-4-fluoro-phenyl)-piperidine. MS esi m/z 332 (M+1)

### Example 44

### 4-(2,6-Dibromo-benzyloxy)-3-(2-methyl-4-fluoro-phenyl)-piperidine

Using Intermediate 11 and 2,6-dibromo-benzyl bromide, and following the same procedure as used in Example 9 gave 4-(2,6-dibromo-benzyloxy)-3-(2-methyl-4-fluoro-phenyl)-piperidine. MS esi m/z 458, 459 (M+1 and M+2)

### Example 45

### 4-(3-chloro-6-methyl-benzyloxy)-3-(2-methyl-4-fluoro-phenyl)-piperidine

Using Intermediate 11 and 3-chloro-6-methyl-benzyl bromide, and following the same procedure as used in Example 9 gave 4-(3-chloro-6-methyl-benzyloxy)-3-(2-methyl-4-fluoro-phenyl)-piperidine. MS esi m/z 348 (M+1)

### Example 46

### 4-(2-iodo-benzyloxy)-3-(2-methyl-4-fluoro-phenyl)-piperidine

Using Intermediate 11 and 2-iodo-benzyl bromide, and following the same procedure as used in Example 9 gave 4-(2-iodo-benzyloxy)-3-(2-methyl-4-fluoro-phenyl)-piperidine. MS esi m/z 426 (M+1)

### Example 47

### 4-(2-isopropyl-benzyloxy)-3-(2-methyl-4-fluoro-phenyl)-piperidine

Using Intermediate 11 and 2-isopropyl-benzyl bromide, and following the same procedure as used in Example 9 gave 4-(2-isopropyl-benzyloxy)-3-(2-methyl-4-fluoro-phenyl)-piperidine. MS esi m/z 342 (M+1)

### Example 48

### 4-(3-fluoro-5-methyl-benzyloxy)-3-(2-methyl-4-fluoro-phenyl)-piperidine

Using Intermediate 11 and 3-fluoro-5-methyl-benzyl bromide, and following the same procedure as used in Example 9 gave 4-(3-fluoro-5-methyl-benzyloxy)-3-(2-methyl-4-fluoro-phenyl)-piperidine. MS esi m/z 332 (M+1)

### Example 49

### 4-[3-(2-methyl-4-fluoro-phenyl)-piperidine-4-yloxymethyl]-3-methoxy-benzoic Acid Ethylester

Using Intermediate 11 and 3-methoxy-4-bromomethyl-benzoic acid ethylester, and following the same procedure as used in Example 9 gave 4-[3-(2-methyl-4-fluoro-phenyl)-piperidine-4-yloxymethyl]-3-methoxy-benzoic acid ethylester. MS esi m/z 402 (M+1)

### Example 50

### 4-benzyloxy-3-(2-methyl-4-fluoro-phenyl)-piperidine

Using Intermediate 11 and benzyl bromide, and following the same procedure as used in Example 9 gave 4-benzyloxy-3-(2-methyl-4-fluoro-phenyl)-piperidine. MS esi m/z 300 (M+1)

### Example 51

### 3-[3-(2-methyl-4-fluoro-phenyl)]-piperidine-4-yloxymethyl-pyridine

Using Intermediate 11 and 3-bromomethyl-pyridine, and following the same procedure as used in Example 9 gave 3-[3-(2-methyl-4-fluoro-phenyl)]-piperidine-4-yloxymethyl-pyridine. MS esi m/z 301 (M+1)

### Example 52

### 3-[3-(2-methyl-4-fluoro-phenyl)]-piperidine-4-yloxymethyl-benzonitrile

Using Intermediate 11 and 3-bromomethyl-benzonitrile, and following the same procedure as used in Example 9 gave 3-[3-(2-methyl-4-fluoro-phenyl)]-piperidine-4-yloxymethyl-benzonitrile. MS esi m/z 325 (M+1)

### Example 53

### 1-[4S-(3,5-bis-trifluoromethyl-benzyloxy)-3R-2-tolyl-piperidine-1-yl]-2-(4-Acetyl-piperazine-1-yl)-ethanone

A solution of 4S-(3,5-bis-trifluoromethyl-benzyloxy)-3R-2-tolyl-piperidine (Example 1) 117 mg (0.257 mmol) was dissolved in CH₂Cl₂ under nitrogen. N-acetyl-piperazine-1-yl-acetic acid (Intermediate 38) 58 mg (0.309 mmol), diisopropylethylamine 0.50 mL (2.57 mmol), and BOP 114 mg (0.257 mmol) were added, and reaction mixture was stirred at room temperature under nitrogen. The reaction mixture was diluted with EtOAc (100 ml) and washed with water three times. The organic layer was dried with MgSO₄. Evaporation of most of the solvent gave 111 mg of oil. This oil was dissolved in about 10 mL of ether and a solution of HCl gas in ether was added drop by drop. The HCl salt was dried under nitrogen on high vacuum for one hour to give 117 mg of 1-[4S-(3,5-bis-trifluoromethyl-benzyloxy)-3R-2-tolyl-piperidine-1-yl]-2-(4-Acetyl-piperazine-1-yl)-ethanone. Fab/MS m/e 586 (M+1); 1H NMR (CD₃Od) δ 2.0 (s, 3H), 2.30(s, 3H), 2.8(m, 4H), 3.0 (m, 4H), 3.2(m, 2H), 3.6(m, 3H), 3.8(m, 2H), 4.22(d, J=12 Hz 1H), 4.4(d J=12 Hz, 1H), 4.7(d, J=7 Hz, 1H), 7.1 (m, 3H), 7.3(d, J=7 Hz, 1H), 7.42(s, 2H), 6.98(s, 1H).

### Example 54

### 1-[4S-(3,5-bis-trifluoromethyl-benzyloxy)-3R-2-tolyl-piperidine-1-yl]-2-pyrrolidin-1-yl-ethanone

Using Example 1 and pyrolidin-1-yl acetic acid, and following the same procedure used in Example 53 gave 1-[4S-(3,5-bis-trifluoromethyl-benzyloxy)-3R-2-tolyl-piperidine-1-yl]-2-pyrrolidin-1-yl-ethanone. Fab/MS m/e 529 (M+1).

### Example 55

### 1-[4-(2-Ethyl-3,5-difluorobenzyloxy)-3-(4-fluoro-2-methyl-phenyl)-piperidine] 2-pyrrolidin-1-yl-ethanone

2-Ethyl-3,5-difluorobenzyl bromide (0.15 mmol, 1.5 equiv) was weighed by CMS into 2-dram vials. The 2-ethyl-3,5-difluorobenzyl bromide was added to 1-tert-Butoxycarbonyl-3R-(2-methyl-4-fluorophenyl)-4S-hydroxypiperidine (30.9 mg, 0.1 mmol, 1 equiv) (Intermediate 11) in 0.6 ml of dry THF with KOt-bu (1.0 M in THF, 0.15 mmol, 1.5 equiv, 0.15 ml). The reaction mixture was agitated and heated for 8 hours at 80° C. in sealed vials.

2 ml water and 2.4 ml EtOAc were then added and the reaction mixture was agitated. The organic layer was removed and and passed through Na₂SO₄ in an SPE cartridge into a tared 2 dram vial. The extraction was repeated twice. The reaction mixture was then dried. To the reactions mixture was then added 1 ml of a 1:1 ratio of TFA:CH₂Cl₂ and the reaction mixture was agitated overnight in sealed vials.

The reaction mixture was evaporated and then 0.5 ml of CH₂Cl₂ were added followed by 2 ml of 2 NaOH and 2.4 ml CH₂Cl₂. The organic layer was separated and loaded onto a conditioned SCX SPE (1 g, 6 ml). The extraction was repeated twice. The extracted product was eluted with MeOH followed by elution with 1 N TEA in MeOH. The solvent was then removed from the reaction mixture.

0.35 ml of DCE and pyrrolidin-1-yl-acetic acid (Intermediate 23) (16.2 mg, 0.125 mmol, 1.25 equiv) were added in 0.2 ml of dry DMF (acid not soluble in DCE), PyBroP (46.6 mg, 0.1 mmol, 1 equiv) in 0.2 ml of dry DCE, and Hunig's base (0.087 ml, 0.5 mmol, 5 equiv) dissolved in 0.2 ml of DCE. The reaction mixture was heated and shaken at 50° C. for 8 hours.

The reaction mixture was partitioned between 2 ml of 1 N NaOH and 2.4 ml of CH₂Cl₂. The organic layer was separated and loaded onto a conditioned SCX SPE (1 g, 6 ml). The extraction was repeated twice. The reaction mixture was eluted with MeOH followed by elution with 1 N TEA in MeOH. Use of DMF to solubilize SM caused product to be eluted in CH₂Cl₂ fraction. The reaction mixture was dried down to yield 1-[4-(2-ethyl-3,5-difluorobenzyloxy)-3-(4-fluoro-2-methyl-phenyl)-piperidine] 2-pyrrolidin-1-yl-ethanone.

All samples contained PyBrop byproducts. Repurification was carried out using 3-ml 500 mg CBA SPE cartridges. Sample was loaded in 2.4 ml of CH₂Cl₂ onto a preconditioned column (2×2.5 ml MeOH, 2×2.5 ml CH₂Cl₂). The sample was rinsed with 2.5 ml of CH₂Cl₂ and then rinsed with 5 ml of MeOH. The sample was then eluted with 5 ml of 1 N TEA in MeOH. The solvent was then removed and the product and PyBrop by product were leeched off a column in CH₂Cl₂ and MeOH fractions. In random checked samples no byproduct were visible in TEA fraction. MS esi m/z 475 (M+1).

### Example 56

### 1-[4-(3,5-dichlorobenzyloxy)-3-(4-fluoro-2-methyl-phenyl)-piperidine]2-pyrrolidin-1-yl-ethanone

Using Intermediate 11 and 3,5-dichloro-benzyl bromide, and following the same procedure used in Example 55 gave 1-[4-(3,5-dichlorobenzyloxy)-3-(4-fluoro-2-methyl-phenyl)-piperidine] 2-pyrrolidin-1-yl-ethanone. MS esi m/z 479 (M+1).

### Example 57

### 1-[4-(3-Methyl-5-fluorobenzyloxy)-3-(4-fluoro-2-methyl-phenyl)piperidine] 2-pyrrolidin-1-yl-ethanone

Using Intermediate 11 and 3-methyl-5-fluorobenzyl bromide, and following the same procedure used in Example 55 gave 1-[4-(3-methyl-5-fluorobenzyloxy)-3-(4-fluoro-2-methyl-phenyl)piperidine] 2-pyrrolidin-1-yl-ethanone. MS esi m/z 443 (M+1).

### Example 58

### 1-[4-(3-Iodo-4-chlorobenzyloxy)-3-(4-fluoro-2-methyl-phenyl)piperidine] 2-pyrrolidin-1-yl-ethanone

Using Intermediate 11 and 3-iodo-4-chlorobenzyl bromide, and following the same procedure used in Example 55 gave 1-[4-(3-iodo-4-chlorobenzyloxy)-3-(4-fluoro-2-methyl-phenyl)piperidine] 2-pyrrolidin-1-yl-ethanone. MS esi m/z 571 (M+1).

### Example 59

### 1-[4-(4,5-dichlorobenzyloxy)-3-(4-fluoro-2-methyl-phenyl)piperidine] 2-pyrrolidin-1-yl-ethanone

Using Intermediate 11 and 3,4-dichloro-benzyl bromide, and following the same procedure used in Example 55 gave 1-[4-(4,5-dichlorobenzyloxy)-3-(4-fluoro-2-methyl-phenyl)piperidine] 2-pyrrolidin-1-yl-ethanone. MS esi m/z 480M+1)

### Example 60

### 1-[4-(2-trifluoromethylbenzyloxy)-3-(4-fluoro-2-methyl-phenyl)piperidine] 2-pyrrolidin-1-yl-ethanone

Using Intermediate 11 and 2-triflouromethyl-benzyl bromide, and following the same procedure used in Example 55 gave 1-[4-(2-trifluoromethylbenzyloxy)-3-(4-fluoro-2-methyl-phenyl)piperidine] 2-pyrrolidin-1-yl-ethanone: MS esi m/z 479 (M+1)

### Example 61

### 1-[4-(5-iodobenzyloxy)-3-(4-fluoro-2-methyl-phenyl)piperidine] 2-pyrrolidin-1-yl-ethanone

Using Intermediate 11 and 3-iodobenzyl bromide, and following the same procedure used in Example 55 gave 1-[4-(5-iodobenzyloxy)-3-(4-fluoro-2-methyl-phenyl)piperidine] 2-pyrrolidin-1-yl-ethanone. MS esi m/z 537 (M+1)

### Example 62

### 1-[4-(Biphenyl-2-ylmethoxy)-3-(4-fluoro-2-methyl-phenyl)piperidine] 2-pyrrolidin-1-yl-ethanone

Using Intermediate 11 and 1-bromomethyl-2-phenylbenzene, and following the same procedure used in Example 55 gave 1-[4-(biphenyl-2-ylmethoxy)-3-(4-fluoro-2-methyl-phenyl)piperidine] 2-pyrrolidin-1-yl-ethanone. MS esi m/z 487 (M+1)

### Example 63

### 1-[4-(2-chloro-5-methoxybenzyloxy)-3-(4-fluoro-2-methyl-phenyl)piperidine] 2-pyrrolidin-1-yl-ethanone

Using Intermediate 11 and 2-chloro-5-methoxybenzyl bromide, and following the same procedure used in Example 55 gave 1-[4-(2-chloro-5-methoxybenzyloxy)-3-(4-fluoro-2-methyl-phenyl)piperidine] 2-pyrrolidin-1-yl-ethanone. MS esi m/z 476 (M+1)

### Example 64

### 1-[4-(2,5-dibromobenzyloxy)-3-(4-fluoro-2-methyl-phenyl)piperidine] 2-pyrrolidin-1-yl-ethanone

Using Intermediate 11 and 2,5-dibromobenzyl bromide, and following the same procedure used in Example 55 gave 1-[4-(2,5-dibromobenzyloxy)-3-(4-fluoro-2-methyl-phenyl)piperidine] 2-pyrrolidin-1-yl-ethanone. MS esi m/z 569 (M+1)

### Example 65

### 1-[4-(2,5-dibromobenzyloxy)-3-(4-fluoro-2-methyl-phenyl)piperidine] 2-pyrrolidin-1-yl-ethanone

Using Intermediate 11 and 2,5-dibromobenzyl bromide, and following the same procedure used in Example 55 gave 1-[4-(2,5-dibromobenzyloxy)-3-(4-fluoro-2-methyl-phenyl)piperidine] 2-pyrrolidin-1-yl-ethanone. MS esi m/z 569 (M+1)

### Example 66

### 1-[4-(4-iodobenzyloxy)-3-(4-fluoro-2-methyl-phenyl)piperidine] 2-pyrrolidin-1-yl-ethanone

Using Intermediate 11 and 4-iodobenzyl bromide, and following the same procedure used in Example 55 gave 1-[4-(4-iodobenzyloxy)-3-(4-fluoro-2-methyl-phenyl)piperidine] 2-pyrrolidin-1-yl-ethanone. MS esi m/z 537 (M+1)

### Example 67

### 1-[4-(5-bromobenzyloxy)-3-(4-fluoro-2-methyl-phenyl)piperidine] 2-pyrrolidin-1-yl-ethanone

Using Intermediate 11 and 3-bromobenzyl bromide, and following the same procedure used in Example 55 gave 1-[4-(5-bromobenzyloxy)-3-(4-fluoro-2-methyl-phenyl)piperidine] 2-pyrrolidin-1-yl-ethanone. MS esi m/z 491 (M+1)

### Example 68

### 1-[4-(2-methyl-5-fluorobenzyloxy)-3-(4-fluoro-2-methyl-phenyl)piperidine] 2-pyrrolidin-1-yl-ethanone

Using Intermediate 11 and 2-methyl-5-fluorobenzyl bromide, and following the same procedure used in Example 55 gave 1-[4-(2-methyl-5-fluorobenzyloxy)-3-(4-fluoro-2-methyl-phenyl)piperidine] 2-pyrrolidin-1-yl-ethanone. MS esi m/z 443 (M+1)

### Example 69

### 1-[4-(3,5-difluorobenzyloxy)-3-(4-fluoro-2-methyl-phenyl)piperidine] 2-pyrrolidin-1-yl-ethanone

Using Intermediate 11 and 3,5-difluorobenzyl bromide, and following the same procedure used in Example 55 gave 1-[4-(3,5-difluorobenzyloxy)-3-(4-fluoro-2-methyl-phenyl)piperidine] 2-pyrrolidin-1-yl-ethanone. MS esi m/z 447 (M+1)

### Example 70

### 1-[4-(2-iodobenzyloxy)-3-(4-fluoro-2-methyl-phenyl)piperidine] 2-pyrrolidin-1-yl-ethanone

Using Intermediate 11 and 2-iodobenzyl bromide, and following the same procedure used in Example 55 gave 1-[4-(2-iodobenzyloxy)-3-(4-fluoro-2-methyl-phenyl)piperidine] 2-pyrrolidin-1-yl-ethanone. MS esi m/z 537 (M+1)

### Example 71

### 1-[4-(2-difluoromethoxybenzyloxy)-3-(4-fluoro-2-methyl-phenyl)piperidine] 2-pyrrolidin-1-yl-ethanone

Using Intermediate 11 and 2-difluoromethoxybenzyl bromide, and following the same procedure used in Example 55 gave 1-[4-(2-difluoromethoxybenzyloxy)-3-(4-fluoro-2-methyl-phenyl)piperidine] 2-pyrrolidin-1-yl-ethanone. MS esi m/z 477 (M+1)

### Example 72

### 1-[4-(2-methyl-5-chlorobenzyloxy)-3-(4-fluoro-2-methyl-phenyl)piperidine] 2-pyrrolidin-1-yl-ethanone

Using Intermediate 11 and 2-methyl-5-chlorobenzyl bromide, and following the same procedure used in Example 55 gave 1-[4-(2-methyl-5-chlorobenzyloxy)-3-(4-fluoro-2-methyl-phenyl)piperidine] 2-pyrrolidin-1-yl-ethanone. MS esi m/z 460 (M+1)

### Example 73

### 1-[4-(3-methylbenzyloxy)-3-(4-fluoro-2-methyl-phenyl)piperidine] 2-pyrrolidin-1-yl-ethanone

Using Intermediate 11 and 3-methylbenzyl bromide, and following the same procedure used in Example 55 gave 1-[4-(3-methylbenzyloxy)-3-(4-fluoro-2-methyl-phenyl)piperidine] 2-pyrrolidin-1-yl-ethanone. MS esi m/z 425 (M+1)

### Example 74

### 1-[4-(2-methyl-3,5-difluorobenzyloxy)-3-(4-fluoro-2-methyl-phenyl)piperidine] 2-pyrrolidin-1-yl-ethanone

Using Intermediate 11 and 2-methyl-3,5-difluorobenzyl bromide, and following the same procedure used in Example 55 gave 1-[4-(2-methyl-3,5-difluorobenzyloxy)-3-(4-fluoro-2-methyl-phenyl)piperidine] 2-pyrrolidin-1-yl-ethanone. MS esi m/z 461 (M+1)

### Example 75

### 1-[4-(3-methoxybenzyloxy)-3-(4-fluoro-2-methyl-phenyl)piperidine] 2-pyrrolidin-1-yl-ethanone

Using Intermediate 11 and 3-methoxybenzyl bromide, and following the same procedure used in Example 55 gave 1-[4-(3-methoxybenzyloxy)-3-(4-fluoro-2-methyl-phenyl)piperidine] 2-pyrrolidin-1-yl-ethanone. MS esi m/z 441 (M+1)

### Example 76

### 1-[4-(3-chlorobenzyloxy)-3-(4-fluoro-2-methyl-phenyl)piperidine] 2-pyrrolidin-1-yl-ethanone

Using Intermediate 11 and 3-chlorobenzyl bromide, and following the same procedure used in Example 55 gave 1-[4-(3-chlorobenzyloxy)-3-(4-fluoro-2-methyl-phenyl)piperidine] 2-pyrrolidin-1-yl-ethanone. MS esi m/z 445 (M+1)

### Example 77

### 1-[4-(3,5-difluorobenzyloxy)-3-(4-fluoro-2-methyl-phenyl)piperidine] 2-pyrrolidin-1-yl-ethanone

Using Intermediate 11 and 3,5-difluorobenzyl bromide, and following the same procedure used in Example 55 gave 1-[4-(3,5-difluorobenzyloxy)-3-(4-fluoro-2-methyl-phenyl)piperidine] 2-pyrrolidin-1-yl-ethanone. MS esi m/z 447 (M+1)

### Example 78

### 1-[4-(2,5-dichlorobenzyloxy)-3-(4-fluoro-2-methyl-phenyl)piperidine] 2-pyrrolidin-1-yl-ethanone

Using Intermediate 11 and 2,5-dichlorobenzyl bromide, and following the same procedure used in Example 55 gave 1-[4-(2,5-dichlorobenzyloxy)-3-(4-fluoro-2-methyl-phenyl)piperidine] 2-pyrrolidin-1-yl-ethanone. MS esi m/z 480 (M+1)

### Example 79

### 1-[4-(2-bromobenzyloxy)-3-(4-fluoro-2-methyl-phenyl)piperidine] 2-pyrrolidin-1-yl-ethanone

Using Intermediate 11 and 2-bromobenzyl bromide, and following the same procedure used in Example 55 gave 1-[4-(2-bromobenzyloxy)-3-(4-fluoro-2-methyl-phenyl)piperidine] 2-pyrrolidin-1-yl-ethanone. MS esi m/z 490, 491 (M+1)

### Example 80

### 1-[4-(4-bromobenzyloxy)-3-(4-fluoro-2-methyl-phenyl)piperidine] 2-pyrrolidin-1-yl-ethanone

Using Intermediate 11 and 4-bromobenzyl bromide, and following the same procedure used in Example 55 gave i-[4-(4-bromobenzyloxy)-3-(4-fluoro-2-methyl-phenyl)piperidine] 2-pyrrolidin-1-yl-ethanone. MS esi m/z 490, 491 (M+1)

### Example 81

### 1-[4-(3,6-difluorobenzyloxy)-3-(4-fluoro-2-methyl-phenyl)piperidine] 2-pyrrolidin-1-yl-ethanone

Using Intermediate 11 and 3,6-difluorobenzyl bromide, and following the same procedure used in Example 55 gave 1-[4-(3,6-difluorobenzyloxy)-3-(4-fluoro-2-methyl-phenyl)piperidine] 2-pyrrolidin-1-yl-ethanone. MS esi m/z 447 (M+1)

### Example 82

### 1-[4-(4-fluorobenzyloxy)-3-(4-fluoro-2-methyl-phenyl)piperidine] 2-pyrrolidin-1-yl-ethanone

Using Intermediate 11 and 4-fluorobenzyl bromide, and following the same procedure used in Example 55 gave 1-[4-(4-fluorobenzyloxy)-3-(4-fluoro-2-methyl-phenyl)piperidine] 2-pyrrolidin-1-yl-ethanone. MS esi m/z 429 (M+1)

### Example 83

### 1-[4-(2-methyl-3,4-difluorobenzyloxy)-3-(4-fluoro-2-methyl-phenyl)piperidine] 2-pyrrolidin-1-yl-ethanone

Using Intermediate 11 and 2-methyl-3,4-difluorobenzyl bromide, and following the same procedure used in Example 55 gave 1-[4-(2-methyl-3,4-difluorobenzyloxy)-3-(4-fluoro-2-methyl-phenyl)piperidine] 2-pyrrolidin-1-yl-ethanone. esi m/z 461 (M+1)

### Example 84

### 1-[4-(2-chlorobenzyloxy)-3-(4-fluoro-2-methyl-phenyl)piperidine] 2-pyrrolidin-1-yl-ethanone

Using Intermediate 11 and 2-chlorobenzyl bromide, and following the same procedure used in Example 55 gave 1-[4-(2-chlorobenzyloxy)-3-(4-fluoro-2-methyl-phenyl)piperidine] 2-pyrrolidin-1-yl-ethanone. MS esi m/z 445 (M+1)

### Example 85

### 1-[4-(2-methyl-3-fluorobenzyloxy)-3-(4-fluoro-2-methyl-phenyl)piperidine] 2-pyrrolidin-1-yl-ethanone

Using Intermediate 11 and 2-methyl-3-fluorobenzyl bromide, and following the same procedure used in Example 55 gave 1-[4-(2-methyl-3-fluorobenzyloxy)-3-(4-fluoro-2-methyl-phenyl)piperidine] 2-pyrrolidin-1-yl-ethanone. MS esi m/z 443 (M+1)

### Example 86

### 1-[4-(4-trifluoromethoxybenzyloxy)-3-(4-fluoro-2-methyl-phenyl)piperidine] 2-pyrrolidin-1-yl-ethanone

Using Intermediate 11 and 4-trifluoromethoxybenzyl bromide, and following the same procedure used in Example 55 gave 1-[4-(4-trifluoromethoxybenzyloxy)-3-(4-fluoro-2-methyl-phenyl)piperidine] 2-pyrrolidin-1-yl-ethanone. MS esi m/z 495 (M+1)

### Example 87

### 1-4-(4-trifluoromethylbenzyloxy)-3-(4-fluoro-2-methyl-phenyl)piperidine] 2-pyrrolidin-1-yl-ethanone

Using Intermediate 11 and 4-trifluoromethylbenzyl bromide, and following the same procedure used in Example 55 gave 1-[4-(4-trifluoromethylbenzyloxy)-3-(4-fluoro-2-methyl-phenyl)piperidine] 2-pyrrolidin-1-yl-ethanone. MS esi m/z 479 (M+1)

### Example 88

### 1-[4-(2-fluorobenzyloxy)-3-(4-fluoro-2-methyl-phenyl)piperidine] 2-pyrrolidin-1-yl-ethanone

Using Intermediate 11 and 2-fluorobenzyl bromide, and following the same procedure used in Example 55 gave 1-[4-(2-fluorobenzyloxy)-3-(4-fluoro-2-methyl-phenyl)piperidine] 2-pyrrolidin-1-yl-ethanone. MS esi m/z 429 (M+1)

### Example 89

### 1-[4-(3,6-difluorobenzyloxy)-3-(4-fluoro-2-methyl-phenyl)piperidine] 2-pyrrolidin-1-yl-ethanone

Using Intermediate 11 and 3,6-difluorobenzyl bromide, and following the same procedure used in Example 55 gave 1-[4-(3,6-difluorobenzyloxy)-3-(4-fluoro-2-methyl-phenyl)piperidine] 2-pyrrolidin-1-yl-ethanone. MS esi m/z 447 (M+1)

### Example 90

### 1-[4-(2-cyanobenzyloxy)-3-(4-fluoro-2-methyl-phenyl)piperidine] 2-pyrrolidin-1-yl-ethanone

Using Intermediate 11 and 2-cyanobenzyl bromide, and following the same procedure used in Example 55 gave 1-[4-(2-cyanobenzyloxy)-3-(4-fluoro-2-methyl-phenyl)piperidine] 2-pyrrolidin-1-yl-ethanone. MS esi m/z 436 (M+1)

### Example 91

### 1-[4-(2,4,6-trifluorobenzyloxy)-3-(4-fluoro-2-methyl-phenyl)piperidine] 2-pyrrolidin-1-yl-ethanone

Using Intermediate 11 and 2,4,6-trifluorobenzyl bromide, and following the same procedure used in Example 55 gave 1-[4-(2,4,6-trifluorobenzyloxy)-3-(4-fluoro-2-methyl-phenyl)piperidine] 2-pyrrolidin-1-yl-ethanone. MS esi m/z 465 (M+1)

### Example 92

### 1-[4-(3-cyanobenzyloxy)-3-(4-fluoro-2-methyl-phenyl)phenyl] 2-pyrrolidin-1-yl-ethanone

Using Intermediate 11 and 3-cyanobenzyl bromide, and following the same procedure used in Example 55 gave 1-[4-(3-cyanobenzyloxy)-3-(4-fluoro-2-methyl-phenyl)phenyl] 2-pyrrolidin-1-yl-ethanone. MS esi m/z 429 (M+1)

### Example 93

### 1-[4-(5-methyl-6-fluorobenzyloxy)-3-(4-fluoro-2-methyl-phenyl)piperidine] 2-pyrrolidin-1-yl-ethanone

Using Intermediate 11 and 3-methyl-2-fluorobenzyl bromide, and following the same procedure used in Example 55 gave 1-[4-(5-methyl-6-fluorobenzyloxy)-3-(4-fluoro-2-methyl-phenyl)piperidine] 2-pyrrolidin-1-yl-ethanone. MS esi m/z 443M+1)

### Example 94

### 1-[4-(4-fluorobenzyloxy)-3-(4-fluoro-2-methyl-phenyl)piperidine] 2-pyrrolidin-1-yl-ethanone

Using Intermediate 11 and 4-fluorobenzyl bromide, and following the same procedure used in Example 55 gave 1-[4-(4-fluorobenzyloxy)-3-(4-fluoro-2-methyl-phenyl)piperidine] 2-pyrrolidin-1-yl-ethanone. MS esi m/z 429M+1)

### Example 95

### 1-[4-(2,6-dichlorobenzyloxy)-3-(4-fluoro-2-methyl-phenyl)piperidine] 2-pyrrolidin-1-yl-ethanone

Using Intermediate 11 and 2,6-dichlorobenzyl bromide, and following the same procedure used in Example 55 gave 1-[4-(2,6-dichlorobenzyloxy)-3-(4-fluoro-2-methyl-phenyl)piperidine] 2-pyrrolidin-1-yl-ethanone. MS esi m/z 480, 481 (M+1)

### Example 96

### 1-[4-(2,5,6-trifluorobenzyloxy)-3-(4-fluoro-2-methyl-phenyl)piperidine] 2-pyrrolidin-1-yl-ethanone

Using Intermediate 11 and 2,3,6-trifluorobenzyl bromide, and following the same procedure used in Example 55 gave 1-[4-(2,5,6-trifluorobenzyloxy)-3-(4-fluoro-2-methyl-phenyl)piperidine] 2-pyrrolidin-1-yl-ethanone. MS esi m/z 465 (M+1)

### Example 97

### 1-[4-(2,6-difluorobenzyloxy)-3-(4-fluoro-2-methyl-phenyl)piperidine] 2-pyrrolidin-1-yl-ethanone

Using Intermediate 11 and 2,6-difluorobenzyl bromide, and following the same procedure used in Example 55 gave 1-[4-(2,6-difluorobenzyloxy)-3-(4-fluoro-2-methyl-phenyl)piperidine] 2-pyrrolidin-1-yl-ethanone. MS esi m/z 447 (M+1)

### Example 98

### 1-[4-(benzyloxy)-3-(4-fluoro-2-methyl-phenyl)piperidine]-2-pyrrolidin-1-yl-ethanone

Using Intermediate 11 and benzyl bromide, and following the same procedure used in Example 55 gave 1-[4-(benzyloxy)-3-(4-fluoro-2-methyl-phenyl)piperidine]-2-pyrrolidin-1-yl-ethanone. MS esi m/z 411 (M+1)

### Example 99

### 1-[(4-fluorophenoxy)benzyloxy-3-(4-fluoro-2-methyl-phenyl)piperidine] 2-pyrrolidin-1-yl-ethanone

Using Intermediate 11 and (4-fluorophenoxy) benzyl bromide, and following the same procedure used in Example 55 gave 1-[(4-fluorophenoxy) benzyloxy-3-(4-fluoro-2-methyl-phenyl)piperidine] 2-pyrrolidin-1-yl-ethanone. MS esi m/z 521 (M+1)

### Example 100

### 1-[4-(2-bromobenzyloxy)-3-(4-fluoro-2-methyl-phenyl)piperidine] 2-pyrrolidin-1-yl-ethanone

Using Intermediate 11 and 2-bromobenzyl bromide, and following the same procedure used in Example 55 gave 1-[4-(2-bromobenzyloxy)-3-(4-fluoro-2-methyl-phenyl)piperidine] 2-pyrrolidin-1-yl-ethanone. MS esi m/z 491, 492 (M+1)

### Example 101

### 1-[4-(3-trifluoromethylbenzyloxy)-3-(4-fluoro-2-methyl-phenyl)piperidine] 2-pyrrolidin-1-yl-ethanone

Using Intermediate 11 and 3-trifluoromethylbenzyl bromide, and following the same procedure used in Example 55 gave 1-[4-(3-trifluoromethylbenzyloxy)-3-(4-fluoro-2-methyl-phenyl)piperidine] 2-pyrrolidin-1-yl-ethanone. MS esi m/z 479 (M+1)

### Example 102

### 1-[(4-Benzoyl-benzyloxy)-3-(4-fluoro-2-methyl-phenyl)piperidine] 2-pyrrolidin-1-yl-ethanone

Using Intermediate 11 and 4-Benzoyl-benzyl bromide, and following the same procedure used in Example 55 gave 1-[(4-Benzoyl-benzyloxy)-3-(4-fluoro-2-methyl-phenyl)piperidine] 2-pyrrolidin-1-yl-ethanone. MS esi m/z 515 (M+1)

### Example 103

### 1-[4S-(2,4-bis-trifluoromethyl-benzyloxy)-3R-2-tolyl-piperidine-1-yl]-2-piperidine-1-yl-ethanone

A solution of 4S-(2,4-bis-trifluoromethyl-benzyloxy)-3R-(2-tolyl)-piperidine (prepared in Example 1) (0.257 mmol) was dissolved in CH₂Cl₂ under nitrogen. Piperidine-1-yl-acetic acid (Intermediate 24) (0.309 mmol) and diisopropylethylamine (2.57 mmol), BOP (0.257 mmol) were added, and the reaction mixture was stirred at room temperature under nitrogen. The reaction mixture was diluted with EtOAc (100 ml) and washed with water three times. The organic layer was dried with MgSO₄. Evaporation of most of the solvent gave 111 mg of oil. This oil was dissolved in about 10 mL of ether and a solution of HCl gas in ether was added drop by drop. The HCl salt was dried under nitrogen on high vacuum for one hour to give 1-[4S-(2,4-bis-trifluoromethyl-benzyloxy)-3R-2-tolyl-piperidine-1-yl]-2-piperidine-1-yl-ethanone. Fab/MS m/e 543 (M+1).

### Example 104

### 1-[4S-(3,5-bis-trifluoromethyl-benzyloxy)-3R-2-tolyl-piperidine-1-yl ]-2-morpholin-1-yl-ethanone

Using 3,5-bis-trifluoromethyl-benzyloxy-3R-2-tolyl-piperidine (Example 1) and morpholineacetic-4-yl-acid (Intermediate 26), and following the procedure in Example 103 gave 1-[4S-(3,5-bis-trifluoromethyl-benzyloxy)-3R-2-tolyl-piperidine-1-yl]-2-morpholin-1-yl-ethanone. Fab/MS m/e 545 (M+1)

### Example 105

### 1-[4S-(3,5-bis-trifluoromethyl-benzyloxy)-3R-2-tolyl-piperidine-1-yl]-2-piperazine-1-yl-ethanone

Using 3,5-bis-trifluoromethyl-benzyloxy-3R-2-tolyl-piperidine (Example 1) and t-BOC-piperazine acetic acid (Intermediate 28), and following the procedure in Example 103 gave 1-[4S-(3,5-bis-trifluoromethyl-benzyloxy)-3R-2-tolyl-piperidine-1-yl]-2-piperazine-1-yl-ethanone. Fab/MS m/e 544 (M+1).

### Example 106

### 1-[4S-(3,5-bis-trifluoromethyl-benzyloxy)-3R-2-tolyl-piperidine-1-yl]-2-(4-methyl-piperazine-1-yl)-ethanone

Using 3,5-bis-trifluoromethyl-benzyloxy-3R-2-tolyl-piperidine (Example 1) and N-methylpiperazineacetic acid acid (Intermediate 30) and following the procedure in Example 103 gave 1-[4S-(3,5-bis-trifluoromethyl-benzyloxy)-3R-2-tolyl-piperidine-1-yl]-2-(4-methyl-piperazine-1-yl)-ethanone. Fab/MS m/e 558 (M+1).

### Example 107

### 1-[4S-(3,5-bis-trifluoromethyl-benzyloxy)-3R-2-tolyl-piperidine-1-yl]-2-(4-ethyl-piperazine-1-yl)-ethanone

Using 3,5-bis-trifluoromethyl-benzyloxy-3R-2-tolyl-piperidine (Example 1) and N-ethylpiperazineacetic acid (Intermediate 32), and following the procedure in Example 103 gave 1-[4S-(3,5-bis-trifluoromethyl-benzyloxy)-3R-2-tolyl-piperidine-1-yl]-2-(4-ethyl-piperazine-1-yl)-ethanone. Fab/MS m/e 572 (M+1).

### Example 108

### 1-[4S-(3,5-bis-trifluoromethyl-benzyloxy)-3R-2-tolyl-piperidine-1-yl]-2-(4-benzyl-piperazine-1-yl)-ethanone

Using 3,5-bis-trifluoromethyl-benzyloxy-3R-2-tolyl-piperidine (Example 1) and N-benzylpiperazineacetic acid (Intermediate 36), and following the procedure in Example 103 gave 1-[4S-(3,5-bis-trifluoromethyl-benzyloxy)-3R-2-tolyl-piperidine-1-yl]-2-(4-benzyl-piperazine-1-yl)-ethanone. Fab/MS m/e 634 (M+1).

### Example 109

### 1-[4S-(3,5-bis-trifluoromethyl-benzyloxy)-3R-2-tolyl-piperidine-1-yl]-2-piperidine-1-carboxylic Acid tert-butyl Ester-1-yl-ethanone

Using 3,5-bis-trifluoromethyl-benzyloxy-3R-2-tolyl-piperidine (Example 1) and 4-Carboxymethyl-piperidine-1-carboxylic acid tert-butyl ester obtained commercially from Aztec or Aldrich, and following the procedure in Example 103 gave 1-[4S-(3,5-bis-trifluoromethyl-benzyloxy)-3R-2-tolyl-piperidine-1-yl]-2-piperidine-1-carboxylic acid tert-butyl ester-1-yl-ethanone. Fab/MS m/e 643 (M+1).

### Example 110

### 1-[4S-(3,5-bis-trifluoromethyl-benzyloxy)-3R-2-tolyl-piperidine-1-yl]-2-piperidine-4-yl-ethanone

The compound of Example 109 was treated with acids such as trifluoroacetic or hydrochloric to give 1-[4S-(3,5-bis-trifluoromethyl-benzyloxy)-3R-2-tolyl-piperidine-1-yl]-2-piperidine-4-yl-ethanone. Fab/MS m/e 543 (M+1).

### Example 111

### 1-[4S-(3,5-bis-trifluoromethyl-benzyloxy)-3R-2-tolyl-piperidine-1-yl]-2-(1-acetyl-piperidine-4-yl)-ethanone

Using 3,5-bis-trifluoromethyl-benzyloxy-3R-2-tolyl-piperidine (Example 1) and 4-acetyl-piperidine-1-carboxylic acid (purchased from Aldrich Chemical Company or Aztec Chemical Company), and following the procedure from example 103 gave 1-[4S-(3,5-bis-trifluoromethyl-benzyloxy)-3R-2-tolyl-piperidine-1-yl]-2-(1-Acetyl-piperidine-4-yl)-ethanone: Fab/MS m/e 585 (M+1).

### Example 112

### 1-[4S-(3,5-bis-trifluoromethyl-benzyloxy)-3R-2-tolyl-piperidine-1-yl]-2-(1-H-imidazole-4-yl)-ethanone

Using 3,5-bis-trifluoromethyl-benzyloxy-3R-2-tolyl-piperidine (Example 1) and and (1H-Imidazol-4-yl)-acetic acid (purchased from Aldrich Chemical Company or Aztec Chemical Company), and following the procedure from example 103 gave 1-[4S-(3,5-bis-trifluoromethyl-benzyloxy)-3R-2-tolyl-piperidine-1-yl]-2-(1-H-imidazole-4-yl)-ethanone. Fab/MS m/e 526 (M+1).

### Example 113

### 1-[4S-(3,5-bis-trifluoromethyl-benzyloxy)-3R-2-tolyl-piperidine-1-yl]-2-pyridine-4-yl-ethanone

Using 3,5-bis-trifluoromethyl-benzyloxy-3R-2-tolyl-piperidine (Example 1) and pyridin-4-yl-acetic acid (purchased from Aldrich Chemical Company or Aztec Chemical Company), and following the procedure in Example 103 gave 1-[4S-(3,5-bis-trifluoromethyl-benzyloxy)-3R-2-tolyl-piperidine-1-yl]-2-Pyridine-4-yl-ethanone. Fab/MS m/e 537 (M+1).

### Example 114

### 1-[4S-(3,5-bis-trifluoromethyl-benzyloxy)-3R-2-tolyl-piperidine-1-yl]-2-pyrrolidin-2-one-1-yl-ethanone

Using 3,5-bis-trifluoromethyl-benzyloxy-3R-2-tolyl-piperidine (Example 1) and (2-Oxo-pyrrolidin-1-yl)-acetic acid (Intermediate 33), and following the procedure in Example 103 gave 1-[4S-(3,5-bis-trifluoromethyl-benzyloxy)-3R-2-tolyl-piperidine-1-yl]-2-pyrrolidin-2-one-1-yl-ethanone Fab/MS m/e 543 (M+1).

### Example 115

### 1-[4S-(3,5-bis-trifluoromethyl-benzyloxy)-3R-2-tolyl-piperidine-1-yl]-2-dimethylamino-1-yl-ethanone

Using 3,5-bis-trifluoromethyl-benzyloxy)-3R-2-tolyl-piperidine (Example 1) and dimethylaminoacetic acid (purchased from Aldrich Chemical Company or Aztec Chemical Company), and following the procedure in Example 103 gave 1-[4S-(3,5-bis-trifluoromethyl-benzyloxy)-3R-2-tolyl-piperidine-1-yl]-2-dimethylamino-1-yl-ethanone. Fab/MS m/e 503 (M+1).

### Example 116

### [4S-(3,5-bis-trifluoromethyl-benzyloxy)-3R-2-tolyl-piperidine-1-yl]-(4-methyl-piperazine-1-yl)-methanone

A solution of 4S-(3,5-bis-trifluoromethyl-benzloxy)-3R-2-tolyl-piperidine (Example 1) 100 mg (0.220 mmol) was dissolved in CH₂Cl₂ under nitrogen. 4-methyl-piperazine carbonyl chloride (purchased from Aldrich Chemical Company or Aztec Chemical Company) 46 mg (0.231 mmol), and diisopropylethylamine 0.4 mL (2.20 mmol) were added, and the reaction mixture was stirred at room temperature under nitrogen. The reaction mixture was diluted with EtOAc (100 ml) and washed with water three times. The organic layer was dried with MgSO₄. Evaporation of most of the solvent gave 120 mg of oil. This oil was dissolved in about 10 mL of ether and a solution of HCl gas in ether was added drop by drop. The HCl salt was dried under nitrogen on high vacuum for one hour to give 125 mg of [4S-(3,5-bis-trifluoromethyl-benzyloxy)-3R-2-tolyl-piperidine-1-yl]-(4-methyl-piperazine-1-yl)-methanone. Fab/MS m/e 544 (M+1)

### Example 117

### [4S-(3,5-bis-trifluoromethyl-benzyloxy)-3R-2-tolyl-piperidine-1-yl]-piperidine-4-methanone

Using 3,5-bis-trifluoromethyl-benzyloxy-3R-2-tolyl-piperidine (Example 1) and piperidine-1,4-dicarboxylic acid mono-tert-butyl ester (purchased form Aldrich), and following the procedure in Example 103 followed by acid treatment gave [4S-(3,5-bis-trifluoromethyl-benzyloxy)-3R-2-tolyl-piperidine-1-yl]-piperidine-4-yl-methanone. Fab/MS m/e 529 (M+1)

### Example 118

### [4S-(3,5-bis-trifluoromethyl-benzyloxy)-3R-2-tolyl-piperidine-1-yl]-piperidine-2-yl-methanone

Using 3,5-bis-trifluoromethyl-benzyloxy-3R-2-tolyl-piperidine (Example 1) and piperidine-1,2-dicarboxylic acid mono-tert-butyl ester, (purchased from Aldrich Chemical Company or Aztec Chemical Company), and following the procedure in Example 103 followed by acid treatment gave [4S-(3,5-bis-trifluoromethyl-benzyloxy)-3R-2-tolyl-piperidine-1-yl]-piperidine-2-yl-methanone. Fab/MS m/e 529 (M+1)

### Example 119

### [4S-(3,5-bis-trifluoromethyl-benzyloxy)-3R-2-tolyl-piperidine-1-yl]-thiazolidin-4-yl-methanone

Using 3,5-bis-trifluoromethyl-benzyloxy-3R-2-tolyl-piperidine (Example 1) and thiazolidine-3,4-dicarboxylic acid 3-tert-butyl ester (purchased from Aldrich Chemical Company or Aztec Chemical Company), and following the procedure in Example 103 followed by acid treatment gave [4S-(3,5-bis-trifluoromethyl-benzyloxy)-3R-2-tolyl-piperidine-1-yl]-thiazolidin-4-yl-methanone.

Fab/MS m/e 533 (M+1)

### Example 120

### [4-(3,5-bis-trifluoromethyl-benzyloxy)-3R-2-tolyl-piperidine-1-yl]-(2-hydroxy-pyridine-3-yl)-methanone

Using 3,5-bis-trifluoromethyl-benzyloxy-3R-2-tolyl-piperidine (Example 1) and 2-Hydroxy-nicotinic acid (purchased form Aldrich), and following the procedure in Example 103 gave [4-(3,5-bis-trifluoromethyl-benzyloxy)-3R-2-tolyl-piperidine-1-yl]-(2-hydroxy-pyridine-3-yl)-methanone. Fab/MS m/e 539 (M+1)

### Example 121

### [4-(3,5-bis-trifluoromethyl-benzyloxy)-3R-2-tolyl-piperidine-1-yl]-(3-hydroxy-pyridine-2-yl)-methanone

Using 3,5-bis-trifluoromethyl-benzyloxy-3R-2-tolyl-piperidine (Example 1) and 3-hydroxy-pyridine-2-carboxylic acid (purchased from Aldrich Chemical Company or Aztec Chemical Company), and following the procedure in Example 103 gave [4-(3,5-bis-trifluoromethyl-benzyloxy)-3R-2-tolyl-piperidine-1-yl]-(3-hydroxy-pyridine-2-yl)-methanone.

Fab/MS m/e 539 (M+1).

### Example 122

### 1-{2-[4S-(3,5-bis-trifluoromethyl-benzyloxy)-3R-2-tolyl-piperidine-1-carbonyl]-4R-hydroxy-pyrrolidine-1-yl)}-ethanone

Using 3,5-bis-trifluoromethyl-benzyloxy-3R-2-tolyl-piperidine (Example 1) and 1-acetyl-4-hydroxy-pyrrolidine-2-carboxylic acid (purchased from Aldrich Chemical Company or Aztec Chemical Company), and following the procedure in Example 103 gave 1-{2-[4S-(3,5-bis-trifluoromethyl-benzyloxy)-3R-2-tolyl-piperidine-1-carbonyl]-4R-hydroxy-pyrrolidine-1-yl}-ethanone. Fab/MS m/e 573 (M+1).

### Example 123

### [4S-(3,5-bis-trifluoromethyl-benzyloxy)-3R-2-tolyl-piperidine-1-yl]-pyridine-4-yl-methanone

Using 3,5-bis-trifluoromethyl-benzyloxy-3R-2-tolyl-piperidine (Example 1) and pyridine-4-carboxylic acid (purchased from Aldrich Chemical Company or Aztec Chemical Company), and following the procedure in Example 103 gave [4S-(3,5-bis-trifluoromethyl-benzyloxy)-3R-2-tolyl-piperidine-1-yl]-pyridine-4-yl-methanone. Fab/MS m/e 523 (M+1)

### Example 124

### 1-{2-[4S-(3,5-bis-trifluoromethyl-benzyloxy)-3R-2-tolyl-piperidine-1-carbonyl]-pyrrolidin-1-yl)}-ethanone

Using 3,5-bis-trifluoromethyl-benzyloxy-3R-2-tolyl-piperidine (Example 1) and 1-acetylpyrrolidine-2-carboxylic acid (purchased from Aldrich Chemical Company or Aztec Chemical Company), and following the procedure in Example 103 gave 1-{2-[4S-(3,5-bis-trifluoromethyl-benzyloxy)-3R-2-tolyl-piperidine-1-carbonyl]-pyrrolidin-1-yl}-ethanone. Fab/MS m/e 557 (M+1).

### Example 125

### [4S-(3,5-bis-trifluoromethyl-benzyloxy)-3R-2-tolyl-piperidine-1-yl]-pyrrolidin-1-yl-methanone

Using 3,5-bis-trifluoromethyl-benzyloxy-3R-2-tolyl-piperidine (Example 1) and pyrrolidine carbonyl chloride (purchased from Aldrich Chemical Company or Aztec Chemical Company), and following the procedure in Example 116 gave [4S-(3,5-bis-trifluoromethyl-benzyloxy)-3R-2-tolyl-piperidine-1-yl]-pyrrolidin-1-yl-methanone. Fab/MS m/e 515 (M+1).

### Example 126

### [4S-(3,5-bis-trifluoromethyl-benzyloxy)-3R-2-tolyl-piperidine-1-yl]-morpholin-4-yl-methanone

Using 3,5-bis-trifluoromethyl-benzyloxy-3R-2-tolyl-piperidine (Example 1) and morpholine carbonyl chloride (purchased from Aldrich Chemical Company or Aztec Chemical Company), and following the procedure in Example 116 gave [4S-(3,5-bis-trifluoromethyl-benzyloxy)-3R-2-tolyl-piperidine-1-yl]-morpholin-4-yl-methanone. Fab/MS m/e 531 (M+1)

### Example 127

### [4S-(3,5-bis-trifluoromethyl-benzyloxy)-3R-2-tolyl-piperidine-1-yl]-2-dimethylamino-ethanone

Using 3,5-bis-trifluoromethyl-benzyloxy-3R-2-tolyl-piperidine (Example 1) and dimethylaminoacetic acid (purchased from Aldrich Chemical Company or Aztec Chemical Company), and following the procedure in Example 103 gave [4S-(3,5-bis-trifluoromethyl-benzyloxy)-3R-2-tolyl-piperidine-1-yl]-2-dimethylamino-ethanone. Fab/MS m/e 503 (M+1)

### Example 128

### N-{2-[4S-(3,5-bis-trifluoromethyl-benzyloxy)-3R-2-tolyl-piperidine-1-yl]-2oxo-ethyl}-acetamide

Using 3,5-bis-trifluoromethyl-benzyloxy-3R-2-tolyl-piperidine (Example 1) and acetylaminoacetic acid (purchased from Aldrich Chemical Company or Aztec Chemical Company), and following the procedure in Example 103 gave N-{2-[4S-(3,5-bis-trifluoromethyl-benzyloxy)-3R-2-tolyl-piperidine-1-yl]-2oxo-ethyl}-acetamide. Fab/MS m/e 517 (M+1)

### Example 129

### 2-Amino-1-[4S-(3,5-bis-trifluoromethyl-benzyloxy)-3R-2-tolyl-piperidine-1-yl]-ethanone

Using 3,5-bis-trifluoromethyl-benzyloxy-3R-2-tolyl-piperidine (Example 1) and t-BOC-glycine (purchased from Aldrich Chemical Company or Aztec Chemical Company), and following the procedure in Example 103 followed by treatment with acid gave 2-Amino-1-[4S-(3,5-bis-trifluoromethyl-benzyloxy)-3R-2-tolyl-piperidine-1-yl]-ethanone. Fab/MS m/e 475 (M+1)

### Example 130

### 2-[4S-(3,5-bis-trifluoromethyl-benzyloxy)-3R-2-tolyl-piperidine-1-yl]-N-N-dimethyl-acetamide

4S-(3,5-bis-trifluoromethyl-benzyloxy)-3R-(2-tolyl)-piperidine (Example 1) (0.00140 mol) was dissolved in CH₂Cl₂ and N,N-dimethyl bromoacetamide (Purchased from Aldrich Chemical Company or Aztec Chemical Company) (0.00143 mol), KOH (0.00146 mol), K₂CO₃ (0.00144 mol) were added and the reaction was stirred at room temperature for three hours. The reaction mixture was then diluted with CH₂Cl₂ (10 ml) and washed with water three times. The organic layer was dried with MgSO₄. Evaporation of most of the solvent gave 2-[4S-(3,5-bis-trifluoromethyl-benzyloxy)-3R-2-tolyl-piperidine-1-yl]-N-N-dimethyl-acetamide. Fab/MS m/e 503 (M+1)

### Example 131

### 4-[4S-(3,5-bis-trifluoromethyl-benzyloxy)-3R-2-tolyl-piperidine-1-carbonyl]-piperidine-2,6-dione

Using 3,5-bis-trifluoromethyl-benzyloxy-3R-2-tolyl-piperidine (Example 1) and 2,6-Dioxo-piperidine-4-carboxylic acid (purchased from Aldrich Chemical Company or Aztec Chemical Company), and following the procedure in Example 103 gave 4-[4S-(3,5-bis-trifluoromethyl-benzyloxy)-3R-2-tolyl-piperidine-1-carbonyl]-piperidine-2,6-dione. Fab/MS m/e 557 (M+1)

### Example 132

### [4S-(3,5-bis-trifluoromethyl-benzyloxy)-3R-2-tolyl-piperidine-1-yl]-pyrrolidin-2-yl-methanone

Using 3,5-bis-trifluoromethyl-benzyloxy-3R-2-tolyl-piperidine (Example 1) and 2,6-dioxo-piperidine-4-carboxylic acid (purchased from Aldrich Chemical Company or Aztec Chemical Company), and following the procedure in Example 103 gave [4S-(3,5-bis-trifluoromethyl-benzyloxy)-3R-2-tolyl-piperidine-1-yl]-pyrrolidin-2-yl-methanone. Fab/MS m/e 515 (M+1).

### Example 133

### [4S-(3,5-bis-trifluoromethyl-benzyloxy)-3R-2-tolyl-piperidine-1-yl]-piperazine-2-yl-methanone

Using 3,5-bis-trifluoromethyl-benzyloxy-3R-2-tolyl-piperidine (Example 1) and piperazine-1,2,4-tricarboxylic acid 1,4-di-tert-butyl ester (purchased from Aldrich Chemical Company or Aztec Chemical Company), and following the procedure in Example 103 followed by treatment with acid gave [4S-(3,5-bis-trifluoromethyl-benzyloxy)-3R-2-tolyl-piperidine-1-yl]-piperazine-2-yl-methanone. Fab/MS m/e 530 (M+1).

### Example 134

### 5-[4S-(3,5-bis-trifluoromethyl-benzyloxy)-3R-2-tolyl-piperidine-1-methyl]-2-4-dihydro-[1.2.4]triazol-3-one

A solution of 4S-(3,5-bis-trifluoromethyl-benzloxy)-3R-2-tolyl-piperidine (Example 1) 100 mg (0.220 mmol) was dissolved in CH₃CN under nitrogen. N-methoxycarbonyl-2-chloroacetamidrazone 73 mg (0.440 mmol) (prepared as in J. Am Chem. Soc., 125, 2129-2135, 2003 and J. Med. Chem, 39, 2907-2914, 1996) and diisopropylethylamine 0.40 mL (2.20 mmol) were added, and the reaction mixture was stirred at room temperature under nitrogen over night. The reaction mixture was diluted with EtOAc (100 ml) and washed with water three times. The organic layer was dried with MgSO₄. Evaporation of most of the solvent gave 111 mg of oil. This oil was dissolved in about 10 mL of xylene and refluxed gently three hours under a nitrogen atmosphere. The reaction mixture was cooled to room temperature and the solvent evaporated to dryness to afford 90 mg of oil. This oil was dissolved in about 10 mL of ether and a solution of HCl gas in ether was added drop by drop. The HCl salt was dried under nitrogen on high vacuum for one hour to give 96 mg of 5-[4S-(3,5-bis-trifluoromethyl-benzyloxy)-3R-2-tolyl-piperidine-1-methyl]-2-4-dihydro-[1,2,4] triazol-3-one. Fab/MS m/e 515 (M+1).

### Example 135

### 5-[4S-(3,5-bis-trifluoromethyl-benzyloxy)-3R-(3,4-difluoro-phenyl)-2-piperidine-1-methyl]-2-4-dihydro-[1.2.4] triazol-3-one

Prepared above compound as described in Example-134 from 4-(3,5-bis-trifluoromethyl-benzyloxy)-3-(3,4-difluoro-phenyl)-piperidine (Example 5). Fab/MS m/e 537 (M+1)

### Example 136

### 5-[4S-(3,5-bis-trifluoromethyl-benzyloxy)-3R-phenyl-2-piperidine-1-methyl]-2-4-dihydro-[1.2.4] triazol-3-one

Prepared above compound as described in Example 134 from 4-(3,5-bis-trifluoromethyl-benzyloxy)-3-phenyl-piperidine (Example 2). Fab/MS m/e 501 (M+1)

### Example 137

### 5-[4S-(3,5-Dimethyl-benzyloxy)-3R-phenyl-2-piperidine-1-methyl]-2-4-dihydro-[1,2.4] triazol-3-one

Prepared above compound as described in Example 134 from 4-(3,5-dimethyl-benzyloxy)-3-phenyl-piperidine (Example 41). Fab/MS m/e 393 (M+1)

### Example 138

### 5-[4-(3,5-bis-trifluoromethyl-benzyloxy)-3-(4-fluoro-phenyl)-2-piperidine-1-methyl]-2-4-dihydro-[1.2.4] triazol-3-one

Prepared above compound as described in Example 134 from 4-(3,5-bis-trifluoromethyl-benzyloxy)-3-(4-fluorophenyl)-piperidine (Example 3). Fab/MS m/e 519 (M+1)

### Example 139

### 5-[4-(3,5-difluoromethyl-benzyloxy)-3-phenyl)-2-piperidine-1-methyl]-2-4-dihydro-[1.2.4] triazol-3-one

Prepared above compound as described in Example 134 from 4-(3,5-difluorobenzyloxy)-3-phenyl-piperidine (Example 19). Fab/MS m/e 401 (M+1)

### Example 140

### 4-(3,5-bis-trifluoromethyl-benzloxy)-1-methyl-3-tolyl-piperidine

A solution of 4S-(3,5-bis-trifluoromethyl-benzloxy)-3R-2-tolyl-piperidine (Example 1) 150 mg (0.330 mmol) was dissolved in THF under nitrogen. NaH 16 mg (0.330 mmol) and methyl iodide 1 mL (0.333 mmol) were added, and the reaction mixture was stirred at room temperature under nitrogen. The reaction mixture was diluted with EtOAc (100 ml) and washed with water three times. The organic layer was dried with MgSO₄. Evaporation of most of the solvent gave 20 mg of oil. This oil was dissolved in about 10 mL of ether and a solution of HCl gas in ether was added drop by drop. The HCl salt was dried under nitrogen on high vacuum for one hour to give 25 mg of 4-(3,5-bis-trifluoromethyl-benzloxy)-1-methyl-3-tolyl-piperidine. Fab/MS m/e 432 (M+1)

### Example 141

### 1-[4-(3,5-bis-trifluoromethyl-benzloxy)-3-(4-fluoro-2-methyl-phenyl)-piperidine-1-yl]-ethanone

A solution of 4-(3,5-bis-trifluoromethyl-benzloxy)-3-(4-fluoro-2-methyl-phenyl)-piperidine (Example 4) prepared as above (4) 185 mg (0.424 mmol) was dissolved in CH₂Cl₂ under nitrogen and TEA 0.6 mL (4.24 mmol), Acetic anhydride 0.3 mL (3.03 mmol) were added, and reaction mixture stirred at room temperature under nitrogen. The reaction mixture diluted with EtOAc (100 ml) and washed two times with water and NaHCO₃. The organic layer was dried with MgSO₄. Evaporation of most of the solvent gave 143 mg of oil. This oil was dissolved in about 10 mL of ether and a solution of HCl gas in ether was added drop by drop. The HCl salt was dried under nitrogen on high vacuum for one hour to give 143 mg of 1-[4-(3,5-bis-trifluoromethyl-benzloxy)-3-(4-fluoro-2-methyl-phenyl)-piperidine-1-yl]-ethanone. Fab/MS m/e 478 (M+1)

### Example 142

### 1-[4S-(3,5-bis-trifluoromethyl-benzyloxy)-3R-2-tolyl-piperidine-1-yl]-ethanone

Prepared above compound as described in Example 141 using the compound prepared from Example 1. Fab/MS m/e 460 (M+1).

### Example 143

### {5-[4-(3,5-bis-trifluoromethyl-benzloxy)-3-phenyl-piperidine-1-yl]-2H[1.2.3] triazol-4-methyl}dimethyl-amine

A solution of 4-(3,5-bis-trifluoromethyl-benzyloxy)-3-phenyl-piperidine (Example 2) 350 mg (0.795 mmol) was dissolved in 5 mL of DME and 5 mL of CH₂Cl₂ under nitrogen and 5-dimethylaminomethyl-2H-[1,2,3] triazole-4-carbaldehyde 368 mg (2.38 mmol) (prepared as in Biorg. and Med. Chem. Lett. 12 (2002) 2515-2518 and J. Med. Chem. 44, 4296-4299, 2001 was added. The reaction mixture was stirred at room temperature over night under nitrogen. The solvent was evaporated to dryness and the residue was dissolved in 10 mL of CH₃OH. NaBH₄ 100 mg (2.38 mmol) was added and the reaction mixture was stirred at room temperature under nitrogen for four hours. The reaction was quenched with saturated citric acid solution. The methanol was removed by rotary evaporation. The water layer was made basic and extracted with EtOAc (200 mL). The combined extracts were washed with water, dried over magnesium sulfate, concentrated under reduced pressure on rotary evaporator to dryness to afford 400 mg of oil. This oil was dissolved in about 10 mL of ether and to this was added to a solution of HCl gas in ether drop by drop. The HCl salt was dried under nitrogen on high vacuum for one hour to give {5-[4-(3,5-bis-trifluoromethyl-benzloxy)-3-phenyl-piperidine-1-yl]-2H[1,2,3] triazol-4-methyl}dimethyl-amine. Fab/MS m/e 542 (M+1)

### Example 144

### {5-[4-(3,5-bis-trifluoromethyl-benzloxy)-3-(4-fluoro-phenyl)-piperidine-1-yl]-2H[1.2.3] triazol-4-methyl} dimethyl-amine

Prepared above compound as described in Example-143 using the compound prepared from Example 3. Fab/MS m/e 560 (M+1).

### Example 145

### {5-[4-(3,5-Dimethyl-benzloxy)-3-phenyl-piperidine-1-yl]-2H[1.2.3] triazol-4-methyl}dimethyl-amine

Prepared above compound as described in Example 143 using the compound prepared from Example 41. Fab/MS m/e 434 (M+1).

### EXAMPLES

### NK-3 Antagonist Compounds

### Example 146

### 3-(4-Fluoro-2-methyl-phenyl)-4-(2-phenyl-butyrylamino)-piperidine

Step 1

A 1-L, three-neck, round bottom flask equipped with a magnetic stirrer and thermometer was purged with nitrogen and charged with anhydrous THF (120 mL), palladium acetate (860 mg 3.83 mmol) and sodium tert-butoxide (11.20 g, 116.5 mmol). The mixture was stirred for 15 min. until the sodium tert-butoxide dissolved. Tri-tert butylphosphine (1.45 g, 7.16 mmol), 2-bromo-5-fluorotoluene (10.50 mL, 83.36 mmol) and 1-tert-butoxycarbonyl-4-piperidone (15.10 g 75.78 mmol) were added, and the reaction was slowly heated at 45-50° C. over a period of 4 hr. The reaction mixture was poured into a solution of sodium bicarbonate (15.0 g) in water (500 mL) and extracted with EtOAc (800 mL). After the organic layer was separated and dried over sodium sulfate, the reaction mixture was concentrated under reduced pressure on a rotary evaporator to dryness to afford 20.0 g of oil. This oil was purified on silica gel flash column and eluted with 15% EtOAc-85% hexane giving 12.8g (56%) of 1-tert-butoxycarbonyl-3-(2-methyl-4-fluoro-phenyl)-4-piperidone as oil which became a solid upon standing at room temperature. GC/MS m/e 307 (M+), RT=4.87 minutes; 1H-NMR (CDCl₃); δ 0.68 (t, 3H), (1.4s, 9H), 2.0(s, 3H), 2.6(m, 2H), 3.3(m, 2H), 3.8(m, 1H), 4.3(m, 2H), 6.8 (m 2H), 7.0(m, 1H).

Step 2

A 1-L, three-neck, round bottom flask equipped with a magnetic stirrer was purged with nitrogen and 1-tert-Butoxycarbonyl-3-(2-methyl-4-fluoro-phenyl)-4-piperidone from step 1 was dissolved in methanol (200 mL). Anhydrous ammonium acetate (64.0 g 830 mmol) and 4A molecular sieves (40.0 g) were added, and the mixture was stirred for one hour. Sodium cyanoborohydride (1.60 g 25.77 mmol) was added, and the reaction was stirred at room temperature for one hour. The reaction mixture was filtered and the filter cake was washed with methanol. The filtrate was concentrated under reduced pressure on a rotary evaporator and the residue was dissolved in EtOAc (500 mL) and washed with water, and sat. sodium chloride solution. The washed residue was dried over sodium sulfate, and then concentrated under reduced pressure on a rotary evaporator to afford 14.0 g of 4-amino-3-(4-fluoro-2-methyl-phenyl)-piperidine-1-carboxylic acid tert-butyl ester as an oil. The racemic amines were purified by silica gel column, eluting with 10% methanol-90% methylene chloride to give 6.0 g of 4-amino-3-(4-fluoro-2-methyl-phenyl)-piperidine-1-carboxylic acid tert-butyl ester. GC/MS m/e 308 (M+), RT=4.86 minutes; 1H-NMR (CDCl₃); δ 1.4 (s, 9H), 2.4(s, 3H), 3.0(m, 2H), 3.43(m, 2H), 6.2(m, 1H), 4.3(m, 2H), 6.8 (m 2H), 7.1(m, 1H).

Step 3

4-amino-3-(4-fluoro-2-methyl-phenyl)-piperidine-1-carboxylic acid tert-butyl ester (452 mg) (1.46 mmol) from step 2 was dissolved in CH₂Cl₂ (10 mL). (S)-(+)-2-phenyl butyric acid (purchased from Aldrich Chemical Company or Aztec Chemical Company) 0.30 mL (1.75 mmol), diisopropylethylamine 1.3 mL (7.30 mmol), and BOP 646 mg (1.46 mmol) were added and the reaction mixture was stirred over night at room temperature under a nitrogen atmosphere. The solvent was removed, the residue was dissolved in EtOAc (50 mL) and washed with water three times, and the organic layer was dried with MgSO₄. The organic solvent was concentrated under reduced pressure on a rotary evaporator to dryness to afford oil. This oil was purified by silica gel column, eluting with 50% EtOAc-50% Hexane gave 416 mg of foam. Fab/MS m/e 355 (M-BOC); 1H-NMR (CDCl3); 0.68(t, 3H), 1.4(S, 9H), 2.6(m, 4H); 2.0(m, 3H); 2.2(S, 3H); 2.6(m, 3H); 2.8(m, 1 H) 2.98(q, 2H); 4.0-4.20(m, 3H); 4.9(d, 1H, J=8 Hz); 6.80(m, 1H); 7.0(d, 1H, J=2 Hz); 7.19(m, 1H); 7.20(m, 4H).

Step 4

A solution of 366 mg (0.805 mmol) of 3-(4-fluoro-2-methyl-phenyl)-4-(2-phenyl-butyrylamino)-piperidine-1-carboxylic acid tert-butyl ester from step 3 was dissolved in 20 mL of CH₂Cl₂. 0.62 mL of TFA were added and the reaction mixture was stirred overnight under a nitrogen atmosphere. The reaction mixture was poured into a saturated NaHCO₃ solution. The reaction mixture was extracted three times with EtOAc and the combined EtOAc extracts were washed with saturated NaCl solution and dried with MgSO₄. Evaporation of solvent gave 351 mg of 3-(4-fluoro-2-methyl-phenyl)-4-(2-phenyl-butyrylamino)-piperidine as an oil. Fab/MS m/e 355 (M+1); 1H NMR (CD₃Od); δ 0.46 (q, 3H), 1.4(m, 2H); 1.7(m, 1H); 1.8(m, 1H); 2.0(m, 1H); 2.30(s, 3H), 3.0 (m, 2H), 3.2(m, 2H), 3.4(m, 2H), 3.98(m, 1H), 4.3(t, 1H), 6.8(m, 2H), 7.2 (m, 4H), 7.3(m, 1H).

### Example 147

### 4-Oxo-2,4-diphenyl-(3-phenyl-piperidin-4-yl)-butyramide

Prepared as in Example 146 using 4-oxo-2,4-diphenyl-butyric acid (purchased from Aldrich Chemical Company or Aztec Chemical Company) as the carboxylic acid in step 3. MS esi m/z 527 (M+1)

### Example 148

### 2-(4-Nitro-phenyl)-(3-phenyl-piperidin-4-yl)-propionamide

Prepared as in Example 146 using 2-(4-nitro-phenyl)-propanoic acid (purchased from Aldrich Chemical Company or Aztec Chemical Company) as the carboxylic acid in step 3. MS esi m/z 468 (M+1)

### Example 149

### 2-Phenyl-(3-phenyl-piperidin-4-yl)-propionamide

Prepared as in Example 146 using 2-phenyl-propanoic acid (purchased from Aldrich Chemical Company or Aztec Chemical Company) as the carboxylic acid in step 3. MS esi m/z 423 (M+1)

### Example 150

### 1,2,3,4-Tetrahydro-naphthalene-1-carboxylic acid (3-phenyl-piperidin-4-yl)-amide

Prepared as in Example 146 using 1,2,3,4-tetrahydro-naphthalene-1-carboxylic acid (purchased from Aldrich Chemical Company or Aztec Chemical Company) as the carboxylic acid in step 3. MS esi m/z 449 (M+1)

### Example 151

### 3-Methyl-2-phenyl-(3-phenyl-piperidin-4-yl)-butyramide

Prepared as in Example 146 using 3-methyl-2-phenyl-butyric acid (purchased from Aldrich Chemical Company or Aztec Chemical Company) as the carboxylic acid in step 3. MS esi m/z 451 (M+1)

### Example 152

### 2-Phenyl-(3-phenyl-piperidin-4-yl)-butyramide

Prepared as in Example 146 using 2-phenyl-butyric acid (purchased from Aldrich Chemical Company or Aztec Chemical Company) as the carboxylic acid in step 3. MS esi m/z 437 (M+1)

### Example 153

### 2-(3-Benzoyl-phenyl)-(3-phenyl-piperidin-4-yl)-propionamide

Prepared as in Example 146 using 2-(3-benzoyl-phenyl)-propanoic acid (purchased from Aldrich Chemical Company or Aztec Chemical Company) as the carboxylic acid in step 3. MS esi m/z 527 (M+1)

### Example 154

### (3-Phenyl-piperidin-4-yl)-2-tolyl-acetamide

Prepared as in Example 146 using 2-tolyl-acetic acid (purchased from Aldrich Chemical Company or Aztec Chemical Company) as the carboxylic acid in step 3. MS esi m/z 423 (M+1)

### Example 155

### (3-Phenyl-piperidin-4-yl)-2-[4-(thiophene-2-carbonyl)-phenyl]-propionamide

Prepared as in Example 146 using 2-[4-(thiophene-2-carbonyl)-phenyl]-propanoic acid (purchased from Aldrich Chemical Company or Aztec Chemical Company) as the carboxylic acid in step 3. MS esi m/z 533 (M+1)

### Example 156

### 6-Fluoro-2-oxo-1,2,3,4-tetrahydro-quinoline-4-carboxylic acid (3-phenyl-piperidin-4-yl)-amide

Prepared as in Example 146 using 6-fluoro-oxo-1,2,3,4 tetrahydroquinoline-4-carboxylic acid (purchased from Aldrich Chemical Company or Aztec Chemical Company) as the carboxylic acid in step 3. MS esi m/z 482 (M+1)

### Example 157

### 3-Furan-2-yl-2-phenyl-(3-phenyl-piperidin-4-yl)-propionamide

Prepared as in Example 146 using 3-furan-2-yl-2-phenyl-propanoic acid (purchased from Aldrich Chemical Company or Aztec Chemical Company) as the carboxylic acid in step 3. MS esi m/z 489 (M+1)

### Example 158

### 6-Chloro-8-methyl-chroman-4-carboxylic acid (3-phenyl-piperidin-4-yl)-amide

Prepared as in Example 146 using 6-chloro-8-methyl-chroman-4-carboxylic acid (purchased from Aldrich Chemical Company or Aztec Chemical Company) as the carboxylic acid in step 3. MS esi m/z 500 (M+2)

### Example 159

### 5-Cyclohexyl-indan-1-carboxylic acid (3-phenyl-piperidin-4-yl)-amide

Prepared as in Example 146 using 5-cyclohexyl-indan-1-carboxylic acid (purchased from Aldrich Chemical Company or Aztec Chemical Company) as the carboxylic acid in step 3. MS esi m/z 517 (M+1)

### Example 160

### 2-(3,4-Dimethoxy-phenyl)-hexanoic acid (3-phenyl-piperidin-4-yl)-amide

Prepared as in Example 146 using 2-(3,4-dimethoxy-phenyl)-hexanoic acid (purchased from Aldrich Chemical Company or Aztec Chemical Company) as the carboxylic acid in step 3. MS esi m/z 525 (M+1)

### Example 161

### 6-Methyl-indan-1-carboxylic acid (3-phenyl-piperidin-4-yl)-amide

Prepared as in Example 146 using 6-methyl-indan-1-carboxylic acid (purchased from Aldrich Chemical Company or Aztec Chemical Company) as the carboxylic acid in step 3. MS esi m/z 449 (M+1)

### Example 162

### 2-[3-Chloro-4-(2,5-dihydro-pyrrol-1-yl)-phenyl-(3-phenyl-piperidin-4-yl]-propionamide

Prepared as in Example 146 using 2-[3-chloro-4-(2,5-dihydro-pyrrol-1-yl)-phenyl-propanoic acid (purchased from Aldrich Chemical Company or Aztec Chemical Company) as the carboxylic acid in step 3. MS esi m/z 525 (M+2)

### Example 163

### 6-Methoxy-3-oxo-indan-1-carboxylic acid (3-phenyl-piperidin-4-yl)-amide

Prepared as in Example 146 using 6-methoxy-3-oxo-indan-1-carboxylic acid (purchased from Aldrich Chemical Company or Aztec Chemical Company) as the carboxylic acid in step 3. MS esi m/z 525 (M+1)

### Example 164

### 6,7-Dichloro-chroman-4-carboxylic acid (3-phenyl-piperidin-4-yl)-amide

Prepared as in Example 146 using 6,7-dichloro-chroman-4-carboxylic acid (purchased from Aldrich Chemical Company or Aztec Chemical Company) as the carboxylic acid in step 3. MS esi m/z 521 (M+2)

### Example 165

### 2-{4-[2-(4-Methoxy-phenyl)-vinyl]-phenyl-(3-phenyl-piperidin-4-yl}-propionamide

Prepared as in Example 146 using 2-{4-[2-(4-Methoxy-phenyl)-vinyl]-phenyl-propanoic acid (purchased from Aldrich Chemical Company or Aztec Chemical Company) as the carboxylic acid in step 3. MS esi m/z 555 (M+1)

### Example 166

### 2-Phenyl-(3-phenyl-piperidin-4-yl)-propionamide

Prepared as in Example 146 using 2-phenyl-propanoic acid (purchased from Aldrich Chemical Company or Aztec Chemical Company) as the carboxylic acid in step 3. MS esi m/z 423 (M+1)

### Example 167

### 2-(4-Chloro-phenyl-3-phenyl-piperidin-4-yl)-propionamide

Prepared as in Example 146 using 2-(4-chloro-phenyl)-propanoic acid (purchased from Aldrich Chemical Company or Aztec Chemical Company) as the carboxylic acid in step 3. MS esi m/z 457 (M+2)

### Example 168

### 2-Phenyl-hexanoic acid (3-phenyl-piperidin-4-yl)-amide

Prepared as in Example 146 using 2-phenyl-hexanoic acid (purchased from Aldrich Chemical Company or Aztec Chemical Company) as the carboxylic acid in step 3. MS esi m/z 465 (M+1)

### Example 169

### Thiochroman-4-carboxylic acid (3-phenyl-piperidin-4-yl)-amide

Prepared as in Example 146 using thiochroman-4-carboxylic acid 2-phenyl-hexanoic acid (purchased from Aldrich Chemical Company or Aztec Chemical Company) as the carboxylic acid in step 3. MS esi m/z 467 (M+1)

### Example 170

### 5-Methoxy-1,2,3,4-tetrahydro-naphthalene-1-carboxylic acid (3-phenyl-piperidin-4-yl)-amide

Prepared as in Example 146 using 5-methoxy-1,2,3,4-tetrahydro-naphthalene-1-carboxylic acid (purchased from Aldrich Chemical Company or Aztec Chemical Company) as the carboxylic acid in step 3. MS esi m/z 479 (M+1)

### Example 171

### 1-Oxo-3-phenyl-1,2,3,4-tetrahydro-isoquinoline-4-carboxylic acid (3-phenyl-piperidin-4-yl)-amide

Prepared as in Example 146 using 1-oxo-3-phenyl-1,2,3,4-tetrahydro-isoquinoline-4-carboxylic acid (purchased from Aldrich Chemical Company or Aztec Chemical Company) as the carboxylic acid in step 3. MS esi m/z 540 (M+1)

### Example 172

### 6-Methoxy-2-methyl-1,2,3,4-tetrahydro-isoquinoline-4-carboxylic acid (3-phenyl-piperidin-4-yl)-amide

Prepared as in Example 146 using 6-methoxy-2-methyl-1,2,3,4-tetrahydro-isoquinoline 4-carboxylic acid 1-oxo-3-phenyl-1,2,3,4-tetrahydro-isoquinoline-4-carboxylic acid (purchased from Aldrich Chemical Company or Aztec Chemical Company) as the carboxylic acid in step 3. MS esi m/z 494 (M+1)

### Example 173

### 2-(4-Hydroxy-phenyl)-3-phenyl-3-phenyl-piperidin-4-yl)-propionamide

Prepared as in Example 146 2-(4-Hydroxy-phenyl)-3-phenyl propanoic acid (purchased from Aldrich Chemical Company or Aztec Chemical Company) as the carboxylic acid in step 3. MS esi m/z 515 (M+1)

### Example 174

### 2-(2-Fluoro-biphenyl-4-yl-3-phenyl-piperidin-4-yl)-propionamide

Prepared as in Example 146 using 2-(2-Fluoro-biphenyl-4-yl propanoic acid (purchased from Aldrich Chemical Company or Aztec Chemical Company) as the carboxylic acid in step 3. MS esi m/z 517 (M+1)

### Example 175

### Chroman-2-carboxylic acid (3-phenyl-piperidin-4-yl)-amide

Prepared as in Example 146 chroman-2-carboxylic acid (purchased from Aldrich Chemical Company or Aztec Chemical Company) as the carboxylic acid in step 3. MS esi m/z 551 (M+1)

### Example 176

### 2,4-Diphenyl-3-phenyl-piperidin-4-yl)-butyramide

Prepared as in Example 146 using 2,4-diphenyl butyric acid (purchased from Aldrich Chemical Company or Aztec Chemical Company) as the carboxylic acid in step 3. MS esi m/z 513 (M+1)

### Example 177

### 6-Chloro-thiochroman-4-carboxylic acid (3-phenyl-piperidin-4-yl)-amide

Prepared as in Example 146 using 6-chloro-thiochroman-4-carboxylic acid (purchased from Aldrich Chemical Company or Aztec Chemical Company) as the carboxylic acid in step 3. MS esi m/z 502 (M+2)

### Example 178

### 7-Methoxy-chroman-4-carboxylic acid (3-phenyl-piperidin-4-yl)-amide

Prepared as in Example 146 using 7-methoxy-chroman-4-carboxylic acid (purchased from Aldrich Chemical Company or Aztec Chemical Company) as the carboxylic acid in step 3. MS esi m/z 481 (M+1)

### Example 179

### 2-[4-(2-Hydroxy-2-methyl-propyl)-phenyl]-(3-phenyl-piperidin-4-yl)-propionamide

Prepared as in Example 146 2-[4-(2-Hydroxy-2-methyl-propyl)-phenyl]-propanoic acid (purchased from Aldrich Chemical Company or Aztec Chemical Company) as the carboxylic acid in step 3. MS esi m/z 495 (M+1)

### Example 180

### 2-Phenyl-(3-phenyl-piperidin-4-yl)-butyramide

Prepared as in Example 146 using 2-Phenyl-butyric acid (purchased from Aldrich Chemical Company or Aztec Chemical Company) as the carboxylic acid in step 3. MS esi m/z 437 (M+1)

### Example 181

### 2-(4-Hydroxy-phenyl-3-phenyl-piperidin-4-yl)-propionamide

Prepared as in Example 146 using 2-(4-Hydroxy-phenyl)-propanoic acid (purchased from Aldrich Chemical Company or Aztec Chemical Company) as the carboxylic acid in step 3. MS esi m/z 439 (M+1)

### Example 182

### 2-Phenyl-3-phenyl-piperidin-4-yl)-acetamide

Prepared as in Example 146 using 2-phenyl acetic acid (purchased from Aldrich Chemical Company or Aztec Chemical Company) as the carboxylic acid in step 3. MS esi m/z 409 (M+1)

### Example 183

### 2,3-Diphenyl-(3-phenyl-piperidin-4-yl)-propionamide

Prepared as in Example 146 using 2,3-diphenyl-propanoic acid (purchased from Aldrich Chemical Company or Aztec Chemical Company) as the carboxylic acid in step 3. MS esi m/z 499 (M+1)

### Example 184

### 2-(3-Phenoxy-phenyl-3-phenyl-piperidin-4-yl)-propionamide

Prepared as in Example 146 using 2-(3-phenoxy-phenyl) propanoic acid (purchased from Aldrich Chemical Company or Aztec Chemical Company) as the carboxylic acid in step 3. MS esi m/z 515 (M+1)

### Example 185

### 2-(4-Isobutyl-phenyl-3-phenyl-piperidin-4-yl)-propionamide

Prepared as in Example 146 using 2-(4-isobutyl-phenyl)-propanoic acid (purchased from Aldrich Chemical Company or Aztec Chemical Company) as the carboxylic acid in step 3. MS esi m/z 479 (M+1)

### Example 186

### 2-Phenyl-3-phenyl-piperidin-4-yl)-propionamide

Prepared as in Example 146 using 2-phenyl-propanoic acid (purchased from Aldrich Chemical Company or Aztec Chemical Company) as the carboxylic acid in step 3. MS esi m/z 423 (M+1)

### Example 187

### Indan-1-carboxylic acid (3-phenyl-piperidin-4-yl)-amide

Prepared as in Example 146 using indan-1-carboxylic acid-carboxylic acid (purchased from Aldrich Chemical Company or Aztec Chemical Company) as the carboxylic acid in step 3. MS esi m/z 423 (M+1)

### Example 188

### 2-Phenoxy-(3-phenyl-piperidin-4-yl)-propionamide

Prepared as in Example 146 using 2-phenoxy propanoic acid (purchased from Aldrich Chemical Company or Aztec Chemical Company) as the carboxylic acid in step 3. MS esi m/z 439 (M+1)

### Example 189

### 3-(4-Methoxy-phenyl)-2-phenyl-(3-phenyl-piperidin-4-yl)-propionamide

Prepared as in Example 146 using 3-(4-Methoxy-phenyl)-2-phenyl-propanoic acid (purchased from Aldrich Chemical Company or Aztec Chemical Company) as the carboxylic acid in step 3. MS esi m/z 529 (M+1)

### Example 190

### 2-Cyclopentyl-2-phenyl-(3-phenyl-piperidin-4-yl)-acetamide

Prepared as in Example 146 using 2-cyclopentyl-2-phenyl acetic acid (purchased from Aldrich Chemical Company or Aztec Chemical Company) as the carboxylic acid in step 3. MS esi m/z 477 (M+1)

### Example 191

### 1,2,3,4-Tetrahydro-isoquinoline-4-carboxylic acid (3-phenyl-piperidin-4-yl)-amide

Prepared as in Example 146 using 1,2,3,4-Tetrahydro-isoquinoline-4-carboxylic acid (purchased from Aldrich Chemical Company or Aztec Chemical Company) as the carboxylic acid in step 3. MS esi m/z 564 (M+1)

### Example 192

### 2-Phenyl-3-(5-phenyl-furan-2-yl-3-phenyl-piperidin-4-yl)-propionamide

Prepared as in Example 146 using 2-phenyl-3-(5-phenyl-furan-2-yl) propanoic acid (purchased from Aldrich Chemical Company or Aztec Chemical Company) as the carboxylic acid in step 3. MS esi m/z 565 (M+1)

### Example 193

### 3-(4-Hydroxy-phenyl)-2-phenyl-3-phenyl-piperidin-4-yl)-propionamide

Prepared as in Example 146 using 3-(4-Hydroxy-phenyl)-2-phenyl propanoic acid (purchased from Aldrich Chemical Company or Aztec Chemical Company) as the carboxylic acid in step 3. MS esi m/z 515 (M+1)

### Example 194

### 6,7-Dichloro-thiochroman-4-carboxylic acid (3-phenyl-piperidin-4-yl)-amide

Prepared as in Example 146 using 6,7-dichloro-thiochroman-4-carboxylic acid (purchased from Aldrich Chemical Company or Aztec Chemical Company) as the carboxylic acid in step 3. MS esi m/z 537 (M+2)

### Example 195

### 6-Fluoro-3-hydroxy-indan-1-carboxylic acid (3-phenyl-piperidin-4-yl)-amide

Prepared as in Example 146 using 6-fluoro-3-hydroxy-indan-1-carboxylic acid (purchased from Aldrich Chemical Company or Aztec Chemical Company) as the carboxylic acid in step 3. MS esi m/z 469 (M+1)

### Example 196

### 4,5,6,7-Tetramethyl-3-oxo-indan-1-carboxylic acid (3-phenyl-piperidin-4-yl)-amide

Prepared as in Example 146 using 4,5,6,7-tetramethyl-3-oxo-indan-1-carboxylic acid (purchased from Aldrich Chemical Company or Aztec Chemical Company) as the carboxylic acid in step 3. MS esi m/z 505 (M+1)

### Example 197

### 2-(2,5-Dimethyl-phenyl)-6-phenyl-hexanoic acid (3-phenyl-piperidin-4-yl)-amide

Prepared as in Example 146 using 2-(2,5-dimethyl-phenyl)-6-phenyl-hexanoic acid (purchased from Aldrich Chemical Company or Aztec Chemical Company) as the carboxylic acid in step 3. MS esi m/z 569 (M+1)

### Example 198

### 3-Methyl-2-phenyl-pentanoic acid (3-phenyl-piperidin-4-yl)-amide

Prepared as in Example 146 3-methyl-2-phenyl-pentanoic acid (purchased from Aldrich Chemical Company or Aztec Chemical Company) as the carboxylic acid in step 3. MS esi m/z 465 (M+1)

### Example 199

### (3-Phenyl-piperidin-4-yl)-2-tolyl-butyramide

Prepared as in Example 146 using 2-tolyl-butyric acid (purchased from Aldrich Chemical Company or Aztec Chemical Company) as the carboxylic acid in step 3. (4), MS esi m/z 451 (M+1)

### Example 200

### 6-Fluoro-chroman-4-carboxylic acid (3-phenyl-piperidin-4-yl)-amide

Prepared as in Example 146 using 6-fluoro-chroman-4-carboxylic acid (purchased from Aldrich Chemical Company or Aztec Chemical Company) as the carboxylic acid in step 3. MS esi m/z 469 (M+1)

### Example 201

### 7-Methoxy-1,2,3,4-tetrahydro-naphthalene-1-carboxylic acid (3-phenyl-piperidin-4-yl)-amide

Prepared as in Example 146 using 7-methoxy-1,2,3,4-tetrahydro-naphthalene-1-carboxylic acid (purchased from Aldrich Chemical Company or Aztec Chemical Company) as the carboxylic acid in step 3. MS esi m/z 479 (M+1)

### Example 202

### 3-Oxo-indan-1-carboxylic acid (3-phenyl-piperidin-4-yl)-amide

Prepared as in Example 146 using 3-oxo-indan-1-carboxylic acid (purchased from Aldrich Chemical Company or Aztec Chemical Company) as the carboxylic acid in step 3. MS esi m/z 449 (M+1)

### Example 203

### 2-Biphenyl-4-yl-pent-4-enoic acid (3-phenyl-piperidin-4-yl)-amide

Prepared as in Example 146 using 2-biphenyl-4-yl-pent-4-enoic acid (purchased from Aldrich Chemical Company or Aztec Chemical Company) as the carboxylic acid in step 3. MS esi m/z 525 (M+1)

### Example 204

### 2-Naphthalen-1-yl-heptanoic acid (3-phenyl-piperidin-4-yl)-amide

Prepared as in Example 146 using 2-Naphthalen-1-yl-heptanoic acid (purchased from Aldrich Chemical Company or Aztec Chemical Company) as the carboxylic acid in step 3. MS esi m/z 529 (M+1)

### Example 205

### 2-(6-Methoxy-naphthalen-2-yl-3-phenyl-piperidin-4-yl)-propionamide

Prepared as in Example 146 using 2-(6-Methoxy-naphthalen-2-yl-propanoic acid (purchased from Aldrich Chemical Company or Aztec Chemical Company) as the carboxylic acid in step 3. esi m/z 503 (M+1)

### Example 206

### 2-(4-Chloro-phenyl)-3-methyl-3-phenyl-piperidin-4-yl)-butyramide

Prepared as in Example 146 using 2-(4-chloro-phenyl)-3-methyl-butyric acid (purchased from Aldrich Chemical Company or Aztec Chemical Company) as the carboxylic acid in step 3. MS esi m/z 486 (M+1)

### Example 207

### 5-Methyl-2-tolyl-hexanoic acid (3-phenyl-piperidin-4-yl)-amide

Prepared as in Example 146 using 5-methyl-2-tolyl-hexanoic acid (purchased from Aldrich Chemical Company or Aztec Chemical Company) as the carboxylic acid in step 3. MS esi m/z 493 (M+1)

### Example 208

### 6-Methoxy-1,2,3,4-tetrahydro-naphthalene-1-carboxylic acid (3-phenyl-piperidin-4-yl)-amide

Prepared as in Example 146 using 6-Methoxy-1,2,3,4-tetrahydro-naphthalene-1-carboxylic acid (purchased from Aldrich Chemical Company or Aztec Chemical Company) as the carboxylic acid in step 3. MS esi m/z 479 (M+1)

### Example 209

### 6-Methoxy-chroman-4-carboxylic acid (3-phenyl-piperidin-4-yl)-amide

Prepared as in Example 146 using 6-methoxy-chroman-4-carboxylic acid (purchased from Aldrich Chemical Company or Aztec Chemical Company) as the carboxylic acid in step 3. MS esi m/z 481 (M+1)

### Example 210

### 6-Fluoro-3-oxo-indan-1-carboxylic acid (3-phenyl-piperidin-4-yl)-amide

Prepared as in Example 146 using 6-fluoro-3-oxo-indan-1-carboxylic acid (purchased from Aldrich Chemical Company or Aztec Chemical Company) as the carboxylic acid in step 3. MS esi m/z 467 (M+1)

### Example 211

### 3-Hydroxy-2-phenyl-(3-phenyl-piperidin-4-yl)-propionamide

Prepared as in Example 146 using 3-hydroxy-2-phenyl-propanoic acid (purchased from Aldrich Chemical Company or Aztec Chemical Company) as the carboxylic acid in step 3. MS esi m/z 439 (M+1)

### Example 212

### 4-(4-Methoxy-phenyl)-4-oxo-2-phenyl-(3-phenyl-piperidin-4-yl)-butyramide

Prepared as in Example 146 using 4-(4-Methoxy-phenyl)-4-oxo-2-phenyl-butyric acid (purchased from Aldrich Chemical Company or Aztec Chemical Company) as the carboxylic acid in step 3. MS esi m/z 557 (M+1)

### Example 213

### 6-Chloro-9-methyl-2,3,4,9-tetrahydro-1-carbazole-4-carboxylic acid (3-phenyl-piperidin-4-yl)-amide

Prepared as in Example 146 using 6-chloro-9-methyl-2,3,4,9-tetrahydro-1-carbazole-4-carboxylic acid (purchased from Aldrich Chemical Company or Aztec Chemical Company) as the carboxylic acid in step 3. MS esi m/z 537 (M+1)

### Example 214

### 2-(4-Isobutyl-phenyl-3-phenyl-piperidin-4-yl)-propionamide

Prepared as in Example 146 using 2-(4-Isobutyl-phenyl-propanoic acid (purchased from Aldrich Chemical Company or Aztec Chemical Company) as the carboxylic acid in step 3. (4), MS esi m/z 479 (M+1)

### Example 215

### 2-Phenoxy-3-phenyl-piperidin-4-yl)-butyramide

Prepared as in Example 146 using 2-phenoxy-butyric acid (purchased from Aldrich Chemical Company or Aztec Chemical Company) as the carboxylic acid in step 3. MS esi m/z 453 (M+1)

### Example 216

### 2-Biphenyl-4-yl-hex-4-enoic acid (3-phenyl-piperidin-4-yl)-amide

Prepared as in Example 146 using 2-biphenyl-4-yl-hex-4-enoic acid (purchased from Aldrich Chemical Company or Aztec Chemical Company) as the carboxylic acid in step 3. MS esi m/z 439 (M+1)

### Example 217

### 2-Cyclohex-2-enyl-2-phenyl-3-phenyl-piperidin-4-yl)-acetamide

Prepared as in Example 146 using 2-cyclohex-2-enyl-2-phenyl-acetic acid (purchased from Aldrich Chemical Company or Aztec Chemical Company) as the carboxylic acid in step 3. MS esi m/z 489 (M+1)

### Example 218

### 6,7-Dimethyl-chroman-4-carboxylic acid (3-phenyl-piperidin-4-yl)-amide

Prepared as in Example 146 using 6,7-Dimethyl-chroman-4-carboxylic acid (purchased from Aldrich Chemical Company or Aztec Chemical Company) as the carboxylic acid in step 3. MS esi m/z 479 (M+1)

### Example 219

### 2-[2-(4-Chloro-phenyl)-benzooxazol-5-yl-3-phenyl-piperidin-4-yl]-propionamide

Prepared as in Example 146 using 2-[2-(4-Chloro-phenyl)-benzooxazol-5-yl]-propanoic acid (purchased from Aldrich Chemical Company or Aztec Chemical Company) as the carboxylic acid in step 3. MS esi m/z 475 (M+1)

### Example 220

### 3-(4-Hydroxy-3,5-diiodo-phenyl)-2-phenyl-3-phenyl-piperidin-4-yl)-propionamide

Prepared as in Example 146 using 3-(4-Hydroxy-3,5-diiodo-phenyl)-2-phenyl-propanoic acid (purchased from Aldrich Chemical Company or Aztec Chemical Company) as the carboxylic acid in step 3. MS esi m/z 767 (M+1)

### Example 221

### 2-(4-Methoxy-phenyl)-3-(5-phenyl-furan-2-yl)-(3-phenyl-piperidin-4-yl)-propionamide

Prepared as in Example 146 using 2-(4-methoxy-phenyl)-3-(5-phenyl-furan-2-yl)-propanoic acid (purchased from Aldrich Chemical Company or Aztec Chemical Company) as the carboxylic acid in step 3. MS esi m/z 595 (M+1)

### Example 222

### 6-Chloro-chroman-4-carboxylic acid (3-phenyl-piperidin-4-yl)-amide

Prepared as in Example 146 using 6-chloro-chroman-4-carboxylic acid (purchased from Aldrich Chemical Company or Aztec Chemical Company) as the carboxylic acid in step 3. MS esi m/z 485 (M+1)

### Example 223

### 4,5-Dimethoxy-indan-1-carboxylic acid (3-phenyl-piperidin-4-yl)-amide (4-77)

Prepared as in Example 146 using 4,5-dimethoxy-indan-1-carboxylic acid (purchased from Aldrich Chemical Company or Aztec Chemical Company) as the carboxylic acid in step 3. MS esi m/z 495 (M+1)

### Example 224

### 6,7-Dichloro-2-methyl-chroman-4-carboxylic acid (3-phenyl-piperidin-4-yl)-amide

Prepared as in Example 146 using 6,7-Dichloro-2-methyl-chroman-4-carboxylic acid (purchased from Aldrich Chemical Company or Aztec Chemical Company) as the carboxylic acid in step 3. MS esi m/z 534 (M+1)

### Example 225

### 2-(6-Hydroxy-naphthalen-2-yl-3-phenyl-piperidin-4-yl)-propionamide

Prepared as in Example 146 using 2-(6-hydroxy-naphthalen-2-yl)-propanoic acid (purchased from Aldrich Chemical Company or Aztec Chemical Company) as the carboxylic acid in step 3. MS esi m/z 489 (M+1)

Based on a reading of the present description and claims, certain modifications to the compounds, compositions and methods described herein will be apparent to one of ordinary skill in the art. The claims appended hereto are intended to encompass these modifications.

1. A compound having the formula I: 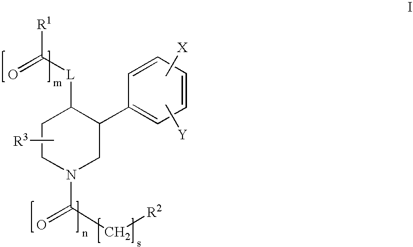
or a pharmaceutically acceptable salt or solvate thereof wherein:
m=0 or 1;
n=0 or 1;
s=0 or 1;
L is —O— or —N(R⁴)—;
R¹ and R² are each independently H, aryl, heteroaryl, (C₁-C₆)alkyl, heterocyloalkyl, —(C₁-C₆)alkylheterocycloalkyl, —(C₁-C₆)alkylheteroaryl, —(C₁-C₆)alkyl-O-aryl, —(C₁-C₆)alkylaryl, and —CH₂N(R⁴)(R⁵), wherein each of said heterocyloalkyl, —(C₁-C₆)alkylheterocycloalkyl, —(C₁-C₆)alkylheteroaryl, —(C₁-C₁₆)alkyl-O-aryl, aryl, —(C₁-C₆)alkylaryl, heteroaryl, and —CH₂N(R⁴)(R⁵), is optionally substituted with 1-3 moieties independently selected from X′, Y′ or Z′;
R³ is H, CF₃, OH, or —(C₁-C₆)alkyl;
R⁴, and R⁵, are each independently selected from H, —(C₁-C₆)alkyl, or —(C₁-C₆)(C═O)R⁷;
R⁷ is (C₁-C₆)alkyl, OH, —N(R⁴)(R⁵), or —OR⁴;
R⁸ and R⁹ are each independently (C₁-C₆)alkyl;
X, Y, X′, Y′ and Z′ are each independently selected from H, —(C₁-C₆)alkyl, —(C₁-C₆)alkyl-NR⁴R⁵, CF₃, OH, —O—(C₁-C₆)alkyl, —(C₁-C₆)alkyl-C(═O)R⁷, aryl, heteroaryl, cycloalkyl, —NO₂, —(C₁-C₆)alkylaryl, —O-aryl, halogen, CN, —CH₃N(R⁴)(R⁵), —C(═O)R⁷, —C(═O)R⁷, —R⁶C(═O)R⁷ or —R⁶C(═O)NR⁴R⁵; and
R⁶ is a bond, —CH₂—, —O—, or —NR⁴—.

2. The compound according to claim 1 wherein
L=O;
n=0 or 1;
m=0;
s=0 or 1;
R¹ and R² are each independently selected from H, CH₃, —(C₁-C₆)alkyl, —CH₂-aryl, —CH₂-heterocycloalkyl, or —CH₂-heteroaryl, wherein each of said —CH₂-aryl, —CH₂-heterocycloalkyl, or —CH₂-heteroaryl is optionally substituted with 1-3 moieties independently selected from X′, Y′ or Z′;
R³ is H;
R⁴ and R⁵ are each independently selected from H, CH₃, or —(C₁-C₆)alkyl; R⁶ is a bond, —CH₂—, —O—, or —NR⁴—;
R⁷ is (C₁-C₆)alkyl, OH, —N(R⁴)(R⁵), or —OR⁴; and
X, Y, X′, Y′ and Z′ are each independently selected from H, (C₁-C₆)alkyl, CF₃, OH, —O—(C₁-C₆)alkyl, halogen, CN, —R⁶C(═O)R⁷ or —R⁶C(═O)NR⁴R⁵.

3. The compound according to claim 1, wherein
L=—NR⁴,
s=0,
n=0 or 1;
m=1,
R¹ and R² are each independently selected from H, CH₃, (C₂-C₆)alkyl, benzyl, —CH₂-heterocycloalkyl, or —CH₂-heteroaryl, wherein each of said benzyl, —CH₂-heterocycloalkyl, or —CH₂-heteroaryl is optionally substituted with 1-3 moieties independently selected from X′, Y′ and Z′;
R³ is H;
R⁴ and R⁵ are each independently selected from H, CH₃, or —(C₁-C₆)alkyl; R⁶ is a bond, —CH₂—, —O—, or —NR⁴—;
R⁷ is (C₁-C₆)alkyl, OH, —N(R⁴)(R⁵), or —OR⁴; and
X, Y, X′, Y′ and Z′ are each independently selected from H, (C₁-C₆)alkyl, CF₃, OH, —O—(C₁-C₆)alkyl, halogen, CN, —R⁶C(═O)R⁷ or —R⁶C(═O)NR⁴R⁵.

4. The compound according to claim 1 selected from the group consisting of
4S-(3,5-bis-trifluoromethyl-benzloxy)-3R-2-tolyl-piperidine
4-(3,5-bis-trifluoromethyl-benzloxy)-3-phenyl-piperidine
4-(3,5-bis-trifluoromethyl-benzyloxy)-3-(4-fluoro-phenyl)-piperidine
4-(3,5-bis-trifluoromethyl-benzyloxy)-3-(2-methyl-4-fluoro-phenyl)-piperidine
4-(3,5-bis-trifluoromethyl-benzyloxy)-3-(3,4-difluoro-phenyl)-piperidine
4-(3,5-bis-trifluoromethyl-benzyloxy)-3-(2-pyridyl)-piperidine
4-(3,5-bis-trifluoromethyl-benzyloxy)-3-(3-chloro-phenyl)-piperidine
4-(4-bromo-benzyloxy)-3-(2-methyl-4-fluoro-phenyl)-piperidine
4-(3-bromo-benzyloxy)-3-(2-methyl-4-fluoro-phenyl)-piperidine
4-(2-bromo-benzyloxy)-3-(2-methyl-4-fluoro-phenyl)-piperidine
4-(2,6-dichloro-benzyloxy)-3-(2-methyl-4-fluoro-phenyl)-piperidine
4-(2,5-dichloro-benzyloxy)-3-(2-methyl-4-fluoro-phenyl)-piperidine
4-(3,5-dichloro-benzyloxy)-3-(2-methyl-4-fluoro-phenyl)-piperidine
4-(4-fluoro-benzyloxy)-3-(2-methyl-4-fluoro-phenyl)-piperidine
4-(2-fluoro-benzyloxy)-3-(2-methyl-4-fluoro-phenyl)-piperidine:
4-(3-fluoro-benzyloxy)-3-(2-methyl-4-fluoro-phenyl)-piperidine
4-(3,4-difluoro-benzyloxy)-3-(2-methyl-4-fluoro-phenyl)-piperidine
4-(3,4-difluoro-benzyloxy)-3-(2-methyl-4-fluoro-phenyl)-piperidine
4-(2,6-di-fluoro-benzyloxy)-3-(2-methyl-4-fluoro-phenyl)-piperidine
4-(3,6-di-fluoro-benzyloxy)-3-(2-methyl-4-fluoro-phenyl)-piperidine
4-(2,4,6-tri-fluoro-benzyloxy)-3-(2-methyl-4-fluoro-phenyl)-piperidine
4-(2,3,6-tri-fluoro-benzyloxy)-3-(2-methyl-4-fluoro-phenyl)-piperidine
4-(4-trifluoromethyl-benzyloxy)-3-(2-methyl-4-fluoro-phenyl)-piperidine
4-(3-trifluoromethyl-benzyloxy)-3-(2-methyl-4-fluoro-phenyl)-piperidine
4-(3,5-bis-trifluoromethyl-benzyloxy)-3-(2-methyl-4-fluoro-phenyl)-piperidine
4-(2,4-bis-trifluoromethyl-benzyloxy)-3-(2-methyl-4-fluoro-phenyl)-piperidine
4-(4-trifluoromethoxyl-benzyloxy)-3-(2-methyl-4-fluoro-phenyl)-piperidine
4-(2-methyl-3,4-Bisfluoromethyl-benzyloxy)-3-(2-methyl-4-fluoro-phenyl)-piperidine
4-(2-methyl-3,5-bis-fluoromethyl-benzyloxy)-3-(2-methyl-4-fluoro-phenyl)-piperidine
4-(2-ethyl-3,5-bis-fluoromethyl-benzyloxy)-3-(2-methyl-4-fluoro-phenyl)-piperidine
4-(2-methyl-benzyloxy)-3-(2-methyl-4-fluoro-phenyl)-piperidine
4-(2-chloro-5-methoxy-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid methylester
4-(3-methoxy-6-bromo-benzyloxy)-3-(2-methyl-4-fluoro-phenyl)-piperidine
4-(3-iodo-4-chloro-benzyloxy)-3-(2-methyl-4-fluoro-phenyl)-piperidine
4-(biphenyl-3-ylmethoxy)-3-(2-methyl-4-fluoro-phenyl)-piperidine
4-[2-(4-fluoro-benzyl)-benzyloxy]-3-(4-fluoro-2-methyl-phenyl)-piperidine
4-(3-methyl-benzyloxy)-3-(2-methyl-4-fluoro-phenyl)-piperidine
4-(2-trifluoromethyl-benzyloxy)-3-(2-methyl-4-fluoro-phenyl)-piperidine
4-(3-4-dimethyl-benzyloxy)-3-(2-methyl-4-fluoro-phenyl)-piperidine
4-(3-methyl-5-fluoro-benzyloxy)-3-(2-methyl-4-fluoro-phenyl)-piperidine
4-(2-methyl-3-fluoro-benzyloxy)-3-(2-methyl-4-fluoro-phenyl)-piperidine
4-(2,6-dibromo-benzyloxy)-3-(2-methyl-4-fluoro-phenyl)-piperidine
4-(3-chloro-6-methyl-benzyloxy)-3-(2-methyl-4-fluoro-phenyl)-piperidine
4-(2-iodo-benzyloxy)-3-(2-methyl-4-fluoro-phenyl)-piperidine
4-(2-isopropyl-benzyloxy)-3-(2-methyl-4-fluoro-phenyl)-piperidine
4-(3-fluoro-5-methyl-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid ethylester
4-benzyloxy-3-(2-methyl-4-fluoro-phenyl)-piperidine
3-[3-(2-methyl-4-fluoro-phenyl)]-piperidine-4-yloxymethyl-pyridine
3-[3-(2-methyl-4-fluoro-phenyl)]-piperidine-4-yloxymethyl-benzonitrile
1-[4S-(3,5-bis-trifluoromethyl-benzyloxy)-3R-2-tolyl-piperidine-1-yl]-2-(4-acetyl-piperazine-1-yl)-ethanone
1-[4S-(3,5-bis-trifluoromethyl-benzyloxy)-3R-2-tolyl-piperidine-1-yl]-2-pyrrolidin-1-yl-ethanone
1-[4-(2-ethyl-3,5-difluorobenzyloxy)-3-(4-fluoro-2-methyl-phenyl)-piperidine] 2-pyrrolidin-1-yl-ethanone
1-[4-(3,5-dichlorobenzyloxy)-3-(4-fluoro-2-methyl-phenyl)-piperidine] 2-pyrrolidin-1-yl-ethanone
1-[4-(3-methyl-5-fluorobenzyloxy)-3-(4-fluoro-2-methyl-phenyl)piperidine] 2-pyrrolidin-1-yl-ethanone
1-[4-(3-iodo-4-chlorobenzyloxy)-3-(4-fluoro-2-methyl-phenyl)piperidine] 2-pyrrolidin-1-yl-ethanone:
1-[4-(4,5-dichlorobenzyloxy)-3-(4-fluoro-2-methyl-phenyl)piperidine] 2-pyrrolidin-1-yl-ethanone
1-[4-(2-trifluoromethylbenzyloxy)-3-(4-fluoro-2-methyl-phenyl)piperidine] 2-pyrrolidin-1-yl-ethanone
1-[4-(5-iodobenzyloxy)-3-(4-fluoro-2-methyl-phenyl)piperidine] 2-pyrrolidin-1-yl-ethanone
1-[4-(biphenyl-2-ylmethoxy)-3-(4-fluoro-2-methyl-phenyl)piperidine] 2-pyrrolidin-1-yl-ethanone:
1-[4-(2-chloro-5-methoxybenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(4-iodobenzyloxy)-3-(4-fluoro-2-methyl-phenyl)piperidine] 2-pyrrolidin-1-yl-ethanone
1-[4-(5-bromobenzyloxy)-3-(4-fluoro-2-methyl-phenyl)piperidine] 2-pyrrolidin-1-yl-ethanone
1-[4-(2-methyl-5-fluorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2-iodobenzyloxy)-3-(4-fluoro-2-methyl-phenyl)piperidine] 2-pyrrolidin-1-yl-ethanone
1-[4-(2-difluoromethoxybenzyloxy)-3-(4-fluoro-2-methyl-phenyl)piperidine] 2-pyrrolidin-1-yl-ethanone:
1-[4-(2-methyl-5-chlorobenzyloxy)-3-(4-fluoro-2-methyl-phenyl)piperidine] 2-pyrrolidin-1-yl-ethanone
1-[4-(3-methylbenzyloxy)-3-(4-fluoro-2-methyl-phenyl)piperidine] 2-pyrrolidin-1-yl-ethanone
1-[4-(2-methyl-3,5-difluorobenzyloxy)-3-(4-fluoro-2-methyl-phenyl)piperidine] 2-pyrrolidin-1-yl-ethanone
1-[4-(3-methoxybenzyloxy)-3-(4-fluoro-2-methyl-phenyl)piperidine] 2-pyrrolidin-1-yl-ethanone
1-[4-(3-chlorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2,5-dichlorobenzyloxy)-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(4-bromobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2-methyl-3,4-difluorobenzyloxy)-3-(4-fluoro-2-methyl-phenyl)piperidine] 2-pyrrolidin-1-yl-ethanone
1-[4-(2-chlorobenzyloxy)-3-(4-fluoro-2-methyl-phenyl)piperidine] 2-pyrrolidin-1-yl-ethanone
1-[4-(2-methyl-3-fluorobenzyloxy)-3-(4-fluoro-2-methyl-phenyl)piperidine] 2-pyrrolidin-1-yl-ethanone
1-[4-(4-trifluoromethoxybenzyloxy)-3-(4-fluoro-2-methyl-phenyl)piperidine] 2-pyrrolidin-1-yl-ethanone
1-[4-(4-trifluoromethylbenzyloxy)-3-(4-fluoro-2-methyl-phenyl)piperidine] 2-pyrrolidin-1-yl-ethanone
1-[4-(2-fluorobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(2-cyanobenzyloxy)-3-(4-fluoro-2-methyl-phenyl)piperidine] 2-pyrrolidin-1-yl-ethanone
1-[4-(2,4,6-trifluorobenzyloxy)-3-(4-fluoro-2-methyl-phenyl)piperidine] 2-pyrrolidin-1-yl-ethanone
1-[4-(3-cyanobenzyloxy)-3-(4-fluoro-2-methyl-phenyl)phenyl] 2-pyrrolidin-1-yl-ethanone
1-[4-(5-methyl-6-fluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2,6-dichlorobenzyloxy)-3-(4-fluoro-2-methyl-phenyl)piperidine] 2-pyrrolidin-1-yl-ethanone
1-[4-(2,5,6-trifluorobenzyloxy)-3-(4-fluoro-2-methyl-phenyl)piperidine] 2-pyrrolidin-1-yl-ethanone
1-[4-(2,6-difluorobenzyloxy)-3-(4-fluoro-2-methyl-phenyl)piperidine] 2-pyrrolidin-1-yl-ethanone
1-[4-(benzyloxy)-3-(4-fluoro-2-methyl-phenyl)piperidine]-2-pyrrolidin-1-yl-ethanone
1-[(4-fluorophenoxy)benzyloxy-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(3-trifluoromethylbenzyloxy)-3-(4-fluoro-2-methyl-phenyl)piperidine] 2-pyrrolidin-1-yl-ethanone
1-[(4-benzoyl-benzyloxy)-3-(4-fluoro-2-methyl-phenyl)piperidine] 2-pyrrolidin-1-yl-ethanone
1-[4S-(2,4-bis-trifluoromethyl-benzyloxy)-3R-2-tolyl-piperidine-1-yl]-2-piperidine-1-yl-ethanone
1-[4S-(3,5-bis-trifluoromethyl-benzyloxy)-3R-2-tolyl-piperidine-1-yl]-2-morpholin-1-yl-ethanone
1-[4S-(3,5-bis-trifluoromethyl-benzyloxy)-3R-2-tolyl-piperidine-1-yl]-2-piperazine-1-yl-ethanone
1-[4S-(3,5-bis-trifluoromethyl-benzyloxy)-3R-2-tolyl-piperidine-1-yl]-2-(4-methyl-piperazine-1-yl)-ethanone
1-[4S-(3,5-bis-trifluoromethyl-benzyloxy)-3R-2-tolyl-piperidine-1-yl]-2-(4-ethyl-piperazine-1-yl)-ethanone
1-[4S-(3,5-bis-trifluoromethyl-benzyloxy)-3R-2-tolyl-piperidine-1-yl]-2-(4-benzyl-piperazine-1-yl)-ethanone
1-[4S-(3,5-bis-trifluoromethyl-benzyloxy)-3R-2-tolyl-piperidine-1-yl]-2-piperidine-1-carboxylic acid tert-butyl ester-1-yl-ethanone
1-[4S-(3,5-bis-trifluoromethyl-benzyloxy)-3R-2-tolyl-piperidine-1-yl]-2-piperidine-4-yl-ethanone
1-[4S-(3,5-bis-trifluoromethyl-benzyloxy)-3R-2-tolyl-piperidine-1-yl]-2-(1-Acetyl-piperidine-4-yl)-ethanone
1-[4S-(3,5-bis-trifluoromethyl-benzyloxy)-3R-2-tolyl-piperidine-1-yl]-2-(1-H-imidazole-4-yl)-ethanone
1-[4S-(3,5-bis-trifluoromethyl-benzyloxy)-3R-2-tolyl-piperidine-1-yl]-2-Pyridine-4-yl-ethanone
1-[4S-(3,5-bis-trifluoromethyl-benzyloxy)-3R-2-tolyl-piperidine-1-yl]-2-pyrrolidin-2-one-1-yl-ethanone
1-[4S-(3,5-bis-trifluoromethyl-benzyloxy)-3R-2-tolyl-piperidine-1-yl]-2-dimethylamino-1-yl-ethanone
[4S-(3,5-bis-trifluoromethyl-benzyloxy)-3R-2-tolyl-piperidine-1-yl]-(4-methyl-piperazine-1-yl)-methanone
[4S-(3,5-bis-trifluoromethyl-benzyloxy)-3R-2-tolyl-piperidine-1-yl]-piperidine-4-yl-methanone
[4S-(3,5-bis-trifluoromethyl-benzyloxy)-3R-2-tolyl-piperidine-1-yl]-piperidine-2-yl-methanone
[4S-(3,5-bis-trifluoromethyl-benzyloxy)-3R-2-tolyl-piperidine-1-yl]-thiazolidin-4-yl-methanone
[4-(3,5-bis-trifluoromethyl-benzyloxy)-3R-2-tolyl-piperidine-1-yl]-(2-hydroxy-pyridine-3-yl)-methanone
[4-(3,5-bis-trifluoromethyl-benzyloxy)-3R-2-tolyl-piperidine-1-yl]-(3-hydroxy-pyridine-2-yl)-methanone
1-{2-[4S-(3,5-bis-trifluoromethyl-benzyloxy)-3R-2-tolyl-piperidine-1-carbonyl]-4R-hydroxy-pyrrolidine-1-yl)}-ethanone
[4S-(3,5-bis-trifluoromethyl-benzyloxy)-3R-2-tolyl-piperidine-1-yl]-pyridine-4-yl-methanone
1-(2-[4S-(3,5-bis-trifluoromethyl-benzyloxy)-3R-2-tolyl-piperidine-1-carbonyl]-pyrrolidin-1-yl)}-ethanone
[4S-(3,5-bis-trifluoromethyl-benzyloxy)-3R-2-tolyl-piperidine-1-yl]-pyrrolidin-1-yl-methanone
[4S-(3,5-bis-trifluoromethyl-benzyloxy)-3R-2-tolyl-piperidine-1-yl]-morpholin-4-yl-methanone
[4S-(3,5-bis-trifluoromethyl-benzyloxy)-3R-2-tolyl-piperidine-1-yl]-2-dimethylamino-ethanone
N-{2-[4S-(3,5-bis-trifluoromethyl-benzyloxy)-3R-2-tolyl-piperidine-1-yl]-2oxo-ethyl}-acetamide
2-amino-1-[4S-(3,5-bis-trifluoromethyl-benzyloxy)-3R-2-tolyl-piperidine-1-yl]-ethanone
2-[4S-(3,5-bis-trifluoromethyl-benzyloxy)-3R-2-tolyl-piperidine-1-yl]-N-N-dimethyl-acetamide
4-[4S-(3,5-bis-trifluoromethyl-benzyloxy)-3R-2-tolyl-piperidine-1-carbonyl]-piperidine-2,6-dione
[4S-(3,5-bis-trifluoromethyl-benzyloxy)-3R-2-tolyl-piperidine-1-yl]-pyrrolidin-2-yl-methanone
[4S-(3,5-bis-trifluoromethyl-benzyloxy)-3R-2-tolyl-piperidine-1-yl]-piperazine-2-yl-methanone
5-[4S-(3,5-bis-trifluoromethyl-benzyloxy)-3R-2-tolyl-piperidine-1-methyl]-2-4-dihydro-[1,2,4] triazol-3-one
5-[4S-(3,5-bis-trifluoromethyl-benzyloxy)-3R-(3,4-difluoro-phenyl)-2-piperidine-1-methyl]-2-4-dihydro-[1,2,4] triazol-3-one
5-[4S-(3,5-bis-trifluoromethyl-benzyloxy)-3R-phenyl-2-piperidine-1-methyl]-2-4-dihydro-[1,2,4] triazol-3-one
5-[4S-(3,5-dimethyl-benzyloxy)-3R-phenyl-2-piperidine-1-methyl]-2-4-dihydro-[1,2,4] triazol-3-one
5-[4-(3,5-bis-trifluoromethyl-benzyloxy)-3-(4-fluoro-phenyl)-2-piperidine-1-methyl]-2-4-dihydro-[1,2,4] triazol-3-one
5-[4-(3,5-bis-fluoromethyl-benzyloxy)-3-phenyl)-2-piperidine-1-methyl]-2-4-dihydro-[1,2,4] triazol-3-one
4-(3,5-bis-trifluoromethyl-benzyloxy)-1-methyl-3-tolyl-piperidine
1-[4-(3,5-bis-trifluoromethyl-benzloxy)-3-(4-fluoro-2-methyl-phenyl)-piperidine-1-yl]-ethanone
1-[4S-(3,5-bis-trifluoromethyl-benzyloxy)-3R-2-tolyl-piperidine-1-yl]-ethanone
{5-[4-(3,5-bis-trifluoromethyl-benzyloxy)-3-phenyl-piperidine-1-yl]-2H[1,2,3] triazol-4-methyl}dimethyl-amine
{5-[4-(3,5-bis-trifluoromethyl-benzyloxy)-3-(4-fluoro-phenyl)-piperidine-1-yl]-2H[1,2,3] triazol-4-methyl} dimethyl-amine
{5-[4-(3,5-dimethyl-benzloxy)-3-phenyl-piperidine-1-yl]-2 H[1,2,3] triazol-4-methyl}dimethyl-amine
3-(4-fluoro-2-methyl-phenyl)-4-(2-phenyl-butyrylamino)-piperidine
4-oxo-2,4-diphenyl-(3-phenyl-piperidin-4-yl)-butyramide
2-(4-nitro-phenyl) -(3-phenyl-piperidin-4-yl)-propionamide
2-phenyl-(3-phenyl-piperidin-4-yl)-propionamide
1,2,3,4-tetrahydro-naphthalene-1-carboxylic acid (3-phenyl-piperidin-4-yl)-amide:
3-methyl-2-phenyl-(3-phenyl-piperidin-4-yl)-butyramide
2-phenyl-(3-phenyl-piperidin-4-yl)-butyramide
2-(3-benzoyl-phenyl)-(3-phenyl-piperidin-4-yl)-propionamide
(3-phenyl-piperidin-4-yl)-2-tolyl-acetamide
(3-phenyl-piperidin-4-yl)-2-[4-(thiophene-2-carbonyl)-phenyl]-propionamide
6-fluoro-2-oxo-1,2,3,4-tetrahydro-quinoline-4-carboxylic acid (3-phenyl-piperidin-4-yl)-amide
3-furan-2-yl-2-phenyl-(3-phenyl-piperidin-4-yl)-propionamide
6-chloro-8-methyl-chroman-4-carboxylic acid (3-phenyl-piperidin-4-yl)-amide
5-cyclohexyl-indan-1-carboxylic acid (3-phenyl-piperidin-4-yl)-amide
2-(3,4-dimethoxy-phenyl)-hexanoic acid (3-phenyl-piperidin-4-yl)-amide
6-methyl-indan-1-carboxylic acid (3-phenyl-piperidin-4-yl)-amide
2-[3-chloro-4-(2,5-dihydro-pyrrol-1-yl)-phenyl-(3-phenyl-piperidin-4-yl)-propionamide
6-methoxy-3-oxo-indan-1-carboxylic acid (3-phenyl-piperidin-4-yl)-amide
6,7-dichloro-chroman-4-carboxylic acid (3-phenyl-piperidin-4-yl)-amide
2-{4-[2-(4-methoxy-phenyl)-vinyl]-phenyl-(3-phenyl-piperidin-4-yl)}-propionamide:
2-phenyl-(3-phenyl-piperidin-4-yl)-propionamide
2-(4-chloro-phenyl-3-phenyl-piperidin-4-yl)-propionamide
2-phenyl-hexanoic acid (3-phenyl-piperidin-4-yl)-amide
thiochroman-4-carboxylic acid (3-phenyl-piperidin-4-yl)-amide
5-methoxy-1,2,3,4-tetrahydro-naphthalene-1-carboxylic acid (3-phenyl-piperidin-4-yl)-amide
1-oxo-3-phenyl-1,2,3,4-tetrahydro-isoquinoline-4-carboxylic acid (3-phenyl-piperidin-4-yl)-amide
6-methoxy-2-methyl-1,2,3,4-tetrahydro-isoquinoline-4-carboxylic acid (3-phenyl-piperidin-4-yl)-amide
2-(4-hydroxy-phenyl)-3-phenyl-3-phenyl-piperidin-4-yl)-propionamide:
2-(2-fluoro-biphenyl-4-yl-3-phenyl-piperidin-4-yl)-propionamide
chroman-2-carboxylic acid (3-phenyl-piperidin-4-yl)-amide
2,4-diphenyl-3-phenyl-piperidin-4-yl)-butyramide
6-chloro-thiochroman-4-carboxylic acid (3-phenyl-piperidin-4-yl)-amide
7-methoxy-chroman-4-carboxylic acid (3-phenyl-piperidin-4-yl)-amide
2-[4-(2-hydroxy-2-methyl-propyl)-phenyl]-(3-phenyl-piperidin-4-yl)-propionamide:
2-phenyl-(3-phenyl-piperidin-4-yl)-butyramide
2-(4-hydroxy-phenyl-3-phenyl-piperidin-4-yl)-propionamide
2-phenyl-3-phenyl-piperidin-4-yl)-acetamide
2,3-diphenyl-(3-phenyl-piperidin-4-yl)-propionamide
2-(3-phenoxy-phenyl-3-phenyl-piperidin-4-yl)-propionamide
2-(4-isobutyl-phenyl-3-phenyl-piperidin-4-yl)-propionamide
2-phenyl-3-phenyl-piperidin-4-yl)-propionamide
indan-1-carboxylic acid (3-phenyl-piperidin-4-yl)-amide
2-phenoxy -(3-phenyl-piperidin-4-yl)-propionamide
3-(4-methoxy-phenyl)-2-phenyl-(3-phenyl-piperidin-4-yl)-propionamide
2-cyclopentyl-2-phenyl-(3-phenyl-piperidin-4-yl)-acetamide
1,2,3,4-tetrahydro-isoquinoline-4-carboxylic acid (3-phenyl-piperidin-4-yl)-amide
2-phenyl-3-(5-phenyl-furan-2-yl-3-phenyl-piperidin-4-yl)-propionamide
3-(4-hydroxy-phenyl)-2-phenyl-3-phenyl-piperidin-4-yl)-propionamide
6,7-dichloro-thiochroman-4-carboxylic acid (3-phenyl-piperidin-4-yl)-amide
6-fluoro-3-hydroxy-indan-1-carboxylic acid (3-phenyl-piperidin-4-yl)-amide
4,5,6,7-tetramethyl-3-oxo-indan-1-carboxylic acid (3-phenyl-piperidin-4-yl)-amide
2-(2,5-dimethyl-phenyl)-6-phenyl-hexanoic acid (3-phenyl-piperidin-4-yl)-amide
3-methyl-2-phenyl-pentanoic acid (3-phenyl-piperidin-4-yl)-amide
(3-phenyl-piperidin-4-yl)-2-tolyl-butyramide
6-fluoro-chroman-4-carboxylic acid (3-phenyl-piperidin-4-yl)-amide
7-methoxy-1,2,3,4-tetrahydro-naphthalene-1-carboxylic acid (3-phenyl-piperidin-4-yl)-amide
3-oxo-indan-1-carboxylic acid (3-phenyl-piperidin-4-yl)-amide
2-biphenyl-4-yl-pent-4-enoic acid (3-phenyl-piperidin-4-yl)-amide
2-naphthalen-1-yl-heptanoic acid (3-phenyl-piperidin-4-yl)-amide
2-(6-methoxy-naphthalen-2-yl-3-phenyl-piperidin-4-yl)-propionamide
2-(4-chloro-phenyl)-3-methyl-3-phenyl-piperidin-4-yl)-butyramide
5-methyl-2-tolyl-hexanoic acid (3-phenyl-piperidin-4-yl)-amide
6-methoxy-1,2,3,4-tetrahydro-naphthalene-1-carboxylic acid (3-phenyl-piperidin-4-yl)-amide
6-methoxy-chroman-4-carboxylic acid (3-phenyl-piperidin-4-yl)-amide
6-fluoro-3-oxo-indan-1-carboxylic acid (3-phenyl-piperidin-4-yl)-amide
3-hydroxy-2-phenyl-(3-phenyl-piperidin-4-yl)-propionamide
4-(4-methoxy-phenyl)-4-oxo-2-phenyl-(3-phenyl-piperidin-4-yl)-butyramide
6-chloro-9-methyl-2,3,4,9-tetrahydro-1-carbazole-4-carboxylic acid (3-phenyl-piperidin-4-yl)-amide
2-(4-isobutyl-phenyl-3-phenyl-piperidin-4-yl)-propionamide
2-phenoxy-3-phenyl-piperidin-4-yl)-butyramide
2-biphenyl-4-yl-hex-4-enoic acid (3-phenyl-piperidin-4-yl)-amide
2-cyclohex-2-enyl-2-phenyl-3-phenyl-piperidin-4-yl)-acetamide
6,7-dimethyl-chroman-4-carboxylic acid (3-phenyl-piperidin-4-yl)-amide
2-[2-(4-chloro-phenyl)-benzooxazol-5-yl-3-phenyl-piperidin-4-yl]-propionamide
3-(4-hydroxy-3,5-diiodo-phenyl)-2-phenyl-3-phenyl-piperidin-4-yl)-propionamide
2-(4-methoxy-phenyl)-3-(5-phenyl-furan-2-yl)-(3-phenyl-piperidin-4-yl)-propionamide
6-chloro-chroman-4-carboxylic acid (3-phenyl-piperidin-4-yl)-amide
4,5-dimethoxy-indan-1-carboxylic acid (3-phenyl-piperidin-4-yl)-amide
6,7-dichloro-2-methyl-chroman-4-carboxylic acid (3-phenyl-piperidin-4-yl)-amide
2-(6-hydroxy-naphthalen-2-yl-3-phenyl-piperidin-4-yl)-propionamide
or pharmaceutically acceptable salts or solvates of said compounds.
The compound according to claim 4 selected from the group consisting of
4S-(3,5-bis-trifluoromethyl-benzyloxy)-3R-2-tolyl-piperidine
4-(3,5-bis-trifluoromethyl-benzloxy)-3-phenyl-piperidine
4-(3,5-bis-trifluoromethyl-benzyloxy)-3-(4-fluoro-phenyl)-piperidine
4-(3,5-bis-trifluoromethyl-benzyloxy)-3-(2-methyl-4-fluoro-phenyl)-piperidine
4-(3,5-bis-trifluoromethyl-benzyloxy)-3-(3,4-difluoro-phenyl)-piperidine
4-(3,5-bis-trifluoromethyl-benzyloxy)-3-(2-pyridyl)-piperidine
4-(3,5-bis-trifluoromethyl-benzyloxy)-3-(3-chloro-phenyl)-piperidine
1-[4S-(3,5-bis-trifluoromethyl-benzyloxy)-3R-2-tolyl-piperidine-1-yl]-2-morpholin-1-yl-ethanone
1-[4S-(3,5-bis-trifluoromethyl-benzyloxy)-3R-2-tolyl-piperidine-1-yl]-2-piperazine-1-yl-ethanone
1-[4S-(3,5-bis-trifluoromethyl-benzyloxy)-3R-2-tolyl-piperidine-1-yl]-2-(4-methyl-piperazine-1-yl)-ethanone
1-[4S-(3,5-bis-trifluoromethyl-benzyloxy)-3R-2-tolyl-piperidine-1-yl]-2-(4-ethyl-piperazine-1-yl)-ethanone
1-[4S-(3,5-bis-trifluoromethyl-benzyloxy)-3R-2-tolyl-piperidine-1-yl]-2-(4-benzyl-piperazine-1-yl)-ethanone
1-[4S-(3,5-bis-trifluoromethyl-benzyloxy)-3R-2-tolyl-piperidine-1-yl]-2-piperidine-1-carboxylic acid tert-butyl ester-1-yl-ethanone
1-[4S-(3,5-bis-trifluoromethyl-benzyloxy)-3R-2-tolyl-piperidine-1-yl]-2-piperidine-4-yl-ethanone
1-[4S-(3,5-bis-trifluoromethyl-benzyloxy)-3R-2-tolyl-piperidine-1-yl]-2-(1-acetyl-piperidine-4-yl)-ethanone
1-[4S-(3,5-bis-trifluoromethyl-benzyloxy)-3R-2-tolyl-piperidine-1-yl]-2-(1-H-imidazole-4-yl)-ethanone
1-[4S-(3,5-bis-trifluoromethyl-benzyloxy)-3R-2-tolyl-piperidine-1-yl]-2-pyridine-4-yl-ethanone
1-[4S-(3,5-bis-trifluoromethyl-benzyloxy)-3R-2-tolyl-piperidine-1-yl]-2-pyrrolidin-2-one-1-yl-ethanone
1-[4S-(3,5-bis-trifluoromethyl-benzyloxy)-3R-2-tolyl-piperidine-1-yl]-2-dimethylamino- 1-yl-ethanone
[4S-(3,5-bis-trifluoromethyl-benzyloxy)-3R-2-tolyl-piperidine-1-yl]-(4-methyl-piperazine-1-yl)-methanone
[4S-(3,5-bis-trifluoromethyl-benzyloxy)-3R-2-tolyl-piperidine-1-yl]-piperidine-4-yl-methanone
[4S-(3,5-bis-trifluoromethyl-benzyloxy)-3R-2-tolyl-piperidine-1-yl]-piperidine-2-yl-methanone
[4S-(3,5-bis-trifluoromethyl-benzyloxy)-3R-2-tolyl-piperidine-1-yl]-pyrrolidin-1-yl-methanone
[4S-(3,5-bis-trifluoromethyl-benzyloxy)-3R-2-tolyl-piperidine-1-yl]-morpholin-4-yl-methanone
[4S-(3,5-bis-trifluoromethyl-benzyloxy)-3R-2-tolyl-piperidine-1-yl]-2-dimethylamino-ethanone
2-[4S-(3,5-bis-trifluoromethyl-benzyloxy)-3R-2-tolyl-piperidine-1-yl]-N-N-dimethyl-acetamide
5-[4S-(3,5-bis-trifluoromethyl-benzyloxy)-3R-2-tolyl-piperidine-1-methyl]-2-4-dihydro-[1,2,4] triazol-3-one
5-[4S-(3,5-bis-trifluoromethyl-benzyloxy)-3R-(3,4-difluoro-phenyl)-2-piperidine-1-methyl]-2-4-dihydro-[1,2,4] triazol-3-one
5-[4S-(3,5-bis-trifluoromethyl-benzyloxy)-3R-phenyl-2-piperidine-1-methyl]-2-4-dihydro-[1,2,4] triazol-3-one
5-[4-(3,5-bis-trifluoromethyl-benzyloxy)-3-(4-fluoro-phenyl)-2-piperidine-1-methyl]-2-4-dihydro-[1,2,4] triazol-3-one
1-[4S-(3,5-bis-trifluoromethyl-benzyloxy)-3R-2-tolyl-piperidine-1-yl]-ethanone
3-(4-fluoro-2-methyl-phenyl)-4-(2-phenyl-butyrylamino)-piperidine
2-phenyl-(3-phenyl-piperidin-4-yl)-propionamide
2,4-diphenyl-3-phenyl-piperidin-4-yl)-butyramide
2-phenyl-(3-phenyl-piperidin-4-yl)-butyramide
2-phenyl-3-phenyl-piperidin-4-yl)-acetamide
2,3-diphenyl-(3-phenyl-piperidin-4-yl)-propionamide
2-(phenyl-3-phenyl-piperidin-4-yl)-propionamide
(3-phenyl-piperidin-4-yl)-2-tolyl-butyramide
and pharmaceutically acceptable salts or solvates thereof.

5. A compound according to claim 4 selected from the group consisting of:
4S-(3,5-bis-trifluoromethyl-benzloxy)-3R-2-tolyl-piperidine
4-(3,5-bis-trifluoromethyl-benzloxy)-3-phenyl-piperidine
4-(3,5-bis-trifluoromethyl-benzyloxy)-3-(4-fluoro-phenyl)-piperidine
4-(3,5-bis-trifluoromethyl-benzyloxy)-3-(2-methyl-4-fluoro-phenyl)-piperidine
4-(3,5-bis-trifluoromethyl-benzyloxy)-3-(3,4-difluoro-phenyl)-piperidine
4-(3,5-bis-trifluoromethyl-benzyloxy)-3-(2-pyridyl)-piperidine
4-(3,5-bis-trifluoromethyl-benzyloxy)-3-(3-chloro-phenyl)-piperidine
4-(4-bromo-benzyloxy)-3-(2-methyl-4-fluoro-phenyl)-piperidine
4-(3-bromo-benzyloxy)-3-(2-methyl-4-fluoro-phenyl)-piperidine
4-(2-bromo-benzyloxy)-3-(2-methyl-4-fluoro-phenyl)-piperidine
4-(2,6-dichloro-benzyloxy)-3-(2-methyl-4-fluoro-phenyl)-piperidine
4-(2,5-dichloro-benzyloxy)-3-(2-methyl-4-fluoro-phenyl)-piperidine
4-(3,5-dichloro-benzyloxy)-3-(2-methyl-4-fluoro-phenyl)-piperidine
4-(4-fluoro-benzyloxy)-3-(2-methyl-4-fluoro-phenyl)-piperidine
4-(2-fluoro-benzyloxy)-3-(2-methyl-4-fluoro-phenyl)-piperidine:
4-(3-fluoro-benzyloxy)-3-(2-methyl-4-fluoro-phenyl)-piperidine
4-(3,4-difluoro-benzyloxy)-3-(2-methyl-4-fluoro-phenyl)-piperidine
4-(3,4-difluoro-benzyloxy)-3-(2-methyl-4-fluoro-phenyl)-piperidine
4-(2,6-di-fluoro-benzyloxy)-3-(2-methyl-4-fluoro-phenyl)-piperidine
4-(3,6-di-fluoro-benzyloxy)-3-(2-methyl-4-fluoro-phenyl)-piperidine
4-(2,4,6-tri-fluoro-benzyloxy)-3-(2-methyl-4-fluoro-phenyl)-piperidine
4-(2,3,6-tri-fluoro-benzyloxy)-3-(2-methyl-4-fluoro-phenyl)-piperidine
4-(4-trifluoromethyl-benzyloxy)-3-(2-methyl-4-fluoro-phenyl)-piperidine
4-(3-trifluoromethyl-benzyloxy)-3-(2-methyl-4-fluoro-phenyl)-piperidine
4-(3,5-bis-trifluoromethyl-benzyloxy)-3-(2-methyl-4-fluoro-phenyl)-piperidine
4-(2,4-bis-trifluoromethyl-benzyloxy)-3-(2-methyl-4-fluoro-phenyl)-piperidine
4-(4-trifluoromethoxyl-benzyloxy)-3-(2-methyl-4-fluoro-phenyl)-piperidine
4-(2-methyl-3,4-Bisfluoromethyl-benzyloxy)-3-(2-methyl-4-fluoro-phenyl)-piperidine
4-(2-methyl-3,5-bis-fluoromethyl-benzyloxy)-3-(2-methyl-4-fluoro-phenyl)-piperidine
4-(2-ethyl-3,5-bis-fluoromethyl-benzyloxy)-3-(2-methyl-4-fluoro-phenyl)-piperidine
4-(2-methyl-benzyloxy)-3-(2-methyl-4-fluoro-phenyl)-piperidine
4-(2-chloro-5-methoxy-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid methylester
4-(3-methoxy-6-bromo-benzyloxy)-3-(2-methyl-4-fluoro-phenyl)-piperidine
4-(3-iodo-4-chloro-benzyloxy)-3-(2-methyl-4-fluoro-phenyl)-piperidine
4-(biphenyl-3-ylmethoxy)-3-(2-methyl-4-fluoro-phenyl)-piperidine
4-[2-(4-fluoro-benzyl)-benzyloxy]-3-(4-fluoro-2-methyl-phenyl)-piperidine
4-(3-methyl-benzyloxy)-3-(2-methyl-4-fluoro-phenyl)-piperidine
4-(2-trifluoromethyl-benzyloxy)-3-(2-methyl-4-fluoro-phenyl)-piperidine
4-(3-4-dimethyl-benzyloxy)-3-(2-methyl-4-fluoro-phenyl)-piperidine
4-(3-methyl-5-fluoro-benzyloxy)-3-(2-methyl-4-fluoro-phenyl)-piperidine
4-(2-methyl-3-fluoro-benzyloxy)-3-(2-methyl-4-fluoro-phenyl)-piperidine
4-(2,6-dibromo-benzyloxy)-3-(2-methyl-4-fluoro-phenyl)-piperidine
4-(3-chloro-6-methyl-benzyloxy)-3-(2-methyl-4-fluoro-phenyl)-piperidine
4-(2-iodo-benzyloxy)-3-(2-methyl-4-fluoro-phenyl)-piperidine
4-(2-isopropyl-benzyloxy)-3-(2-methyl-4-fluoro-phenyl)-piperidine
4-(3-fluoro-5-methyl-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid ethylester
4-benzyloxy-3-(2-methyl-4-fluoro-phenyl)-piperidine
3-[3-(2-methyl-4-fluoro-phenyl)]-piperidine-4-yloxymethyl-pyridine
3-[3-(2-methyl-4-fluoro-phenyl)]-piperidine-4-yloxymethyl-benzonitrile
1-[4S-(3,5-bis-trifluoromethyl-benzyloxy)-3R-2-tolyl-piperidine-1-yl]-2-(4-acetyl-piperazine-1-yl)-ethanone
1-[4S-(3,5-bis-trifluoromethyl-benzyloxy)-3R-2-tolyl-piperidine-1-yl]-2-pyrrolidin-1-yl-ethanone
1-[4-(2-ethyl-3,5-difluorobenzyloxy)-3-(4-fluoro-2-methyl-phenyl)-piperidine] 2-pyrrolidin-1-yl-ethanone
1-[4-(3,5-dichlorobenzyloxy)-3-(4-fluoro-2-methyl-phenyl)-piperidine] 2-pyrrolidin-1-yl-ethanone
1-[4-(3-methyl-5-fluorobenzyloxy)-3-(4-fluoro-2-methyl-phenyl)piperidine] 2-pyrrolidin-1-yl-ethanone
1-[4-(3-iodo-4-chlorobenzyloxy)-3-(4-fluoro-2-methyl-phenyl)piperidine] 2-pyrrolidin-1-yl-ethanone:
1-[4-(4,5-dichlorobenzyloxy)-3-(4-fluoro-2-methyl-phenyl)piperidine] 2-pyrrolidin-1-yl-ethanone
1-[4-(2-trifluoromethylbenzyloxy)-3-(4-fluoro-2-methyl-phenyl)piperidine] 2-pyrrolidin-1-yl-ethanone
1-[4-(5-iodobenzyloxy)-3-(4-fluoro-2-methyl-phenyl)piperidine] 2-pyrrolidin-1-yl-ethanone
1-[4-(biphenyl-2-ylmethoxy)-3-(4-fluoro-2-methyl-phenyl)piperidine] 2-pyrrolidin-1-yl-ethanone:
1-[4-(2-chloro-5-methoxybenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(4-iodobenzyloxy)-3-(4-fluoro-2-methyl-phenyl)piperidine] 2-pyrrolidin-1-yl-ethanone
1-[4-(5-bromobenzyloxy)-3-(4-fluoro-2-methyl-phenyl)piperidine] 2-pyrrolidin-1-yl-ethanone
1-[4-(2-methyl-5-fluorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2-iodobenzyloxy)-3-(4-fluoro-2-methyl-phenyl)piperidine] 2-pyrrolidin-1-yl-ethanone
1-[4-(2-difluoromethoxybenzyloxy)-3-(4-fluoro-2-methyl-phenyl)piperidine] 2-pyrrolidin-1-yl-ethanone:
1-[4-(2-methyl-5-chlorobenzyloxy)-3-(4-fluoro-2-methyl-phenyl)piperidine] 2-pyrrolidin-1-yl-ethanone
1-[4-(3-methylbenzyloxy)-3-(4-fluoro-2-methyl-phenyl)piperidine] 2-pyrrolidin-1-yl-ethanone
1-[4-(2-methyl-3,5-difluorobenzyloxy)-3-(4-fluoro-2-methyl-phenyl)piperidine] 2-pyrrolidin-1-yl-ethanone
1-[4-(3-methoxybenzyloxy)-3-(4-fluoro-2-methyl-phenyl)piperidine] 2-pyrrolidin-1-yl-ethanone
1-[4-(3-chlorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2,5-dichlorobenzyloxy)-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(4-bromobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2-methyl-3,4-difluorobenzyloxy)-3-(4-fluoro-2-methyl-phenyl)piperidine] 2-pyrrolidin-1-yl-ethanone
1-[4-(2-chlorobenzyloxy)-3-(4-fluoro-2-methyl-phenyl)piperidine] 2-pyrrolidin-1-yl-ethanone
1-[4-(2-methyl-3-fluorobenzyloxy)-3-(4-fluoro-2-methyl-phenyl)piperidine] 2-pyrrolidin-1-yl-ethanone
1-[4-(4-trifluoromethoxybenzyloxy)-3-(4-fluoro-2-methyl-phenyl)piperidine] 2-pyrrolidin-1-yl-ethanone
1-[4-(4-trifluoromethylbenzyloxy)-3-(4-fluoro-2-methyl-phenyl)piperidine] 2-pyrrolidin-1-yl-ethanone
1-[4-(2-fluorobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(2-cyanobenzyloxy)-3-(4-fluoro-2-methyl-phenyl)piperidine] 2-pyrrolidin-1-yl-1 0 ethanone
1-[4-(2,4,6-trifluorobenzyloxy)-3-(4-fluoro-2-methyl-phenyl)piperidine] 2-pyrrolidin-1-yl-ethanone
1-[4-(3-cyanobenzyloxy)-3-(4-fluoro-2-methyl-phenyl)phenyl] 2-pyrrolidin-1-yl-ethanone
1-[4-(5-methyl-6-fluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2,6-dichlorobenzyloxy)-3-(4-fluoro-2-methyl-phenyl)piperidine] 2-pyrrolidin-1-yl-ethanone
1-[4-(2,5,6-trifluorobenzyloxy)-3-(4-fluoro-2-methyl-phenyl)piperidine] 2-pyrrolidin-1-yl-ethanone
1-[4-(2,6-difluorobenzyloxy)-3-(4-fluoro-2-methyl-phenyl)piperidine] 2-pyrrolidin-1-yl-ethanone
1-[4-(benzyloxy)-3-(4-fluoro-2-methyl-phenyl)piperidine]-2-pyrrolidin-1-yl-ethanone
1-[(4-fluorophenoxy) benzyloxy-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(3-trifluoromethylbenzyloxy)-3-(4-fluoro-2-methyl-phenyl)piperidine] 2-pyrrolidin-1-yl-ethanone
1-[(4-benzoyl-benzyloxy)-3-(4-fluoro-2-methyl-phenyl)piperidine] 2-pyrrolidin-1-yl-ethanone
1-[4S-(2,4-bis-trifluoromethyl-benzyloxy)-3R-2-tolyl-piperidine-1-yl]-2-piperidine-1-yl-ethanone
1-[4S-(3,5-bis-trifluoromethyl-benzyloxy)-3R-2-tolyl-piperidine-1-yl]-2-morpholin-1-yl-ethanone
1-[4S-(3,5-bis-trifluoromethyl-benzyloxy)-3R-2-tolyl-piperidine-1-yl]-2-piperazine-1-yl-ethanone
1-[4S-(3,5-bis-trifluoromethyl-benzyloxy)-3R-2-tolyl-piperidine-1-yl]-2-(4-methyl-piperazine-1-yl)-ethanone
1-[4S-(3,5-bis-trifluoromethyl-benzyloxy)-3R-2-tolyl-piperidine-1-yl]-2-(4-ethyl-piperazine-1-yl)-ethanone
1-[4S-(3,5-bis-trifluoromethyl-benzyloxy)-3R-2-tolyl-piperidine-1-yl]-2-(4-benzyl-piperazine-1-yl)-ethanone
1-[4S-(3,5-bis-trifluoromethyl-benzyloxy)-3R-2-tolyl-piperidine-1-yl]-2-piperidine-1-carboxylic acid tert-butyl ester-1-yl-ethanone
1-[4S-(3,5-bis-trifluoromethyl-benzyloxy)-3R-2-tolyl-piperidine-1-yl]-2-piperidine-4-yl-ethanone
1-[4S-(3,5-bis-trifluoromethyl-benzyloxy)-3R-2-tolyl-piperidine-1-yl]-2-(1-Acetyl-piperidine-4-yl)-ethanone
1-[4S-(3,5-bis-trifluoromethyl-benzyloxy)-3R-2-tolyl-piperidine-1-yl]-2-(1-H-imidazole-4-yl)-ethanone
1-[4S-(3,5-bis-trifluoromethyl-benzyloxy)-3R-2-tolyl-piperidine-1-yl]-2-Pyridine-4-yl-ethanone
1-[4S-(3,5-bis-trifluoromethyl-benzyloxy)-3R-2-tolyl-piperidine-1-yl]-2-pyrrolidin-2-one-1-yl-ethanone
1-[4S-(3,5-bis-trifluoromethyl-benzyloxy)-3R-2-tolyl-piperidine-1-yl]-2-dimethylamino-1-yl-ethanone
[4S-(3,5-bis-trifluoromethyl-benzyloxy)-3R-2-tolyl-piperidine-1-yl]-(4-methyl-piperazine-1-yl)-methanone
[4S-(3,5-bis-trifluoromethyl-benzyloxy)-3R-2-tolyl-piperidine-1-yl]-piperidine-4-yl-methanone
[4S-(3,5-bis-trifluoromethyl-benzyloxy)-3R-2-tolyl-piperidine-1-yl]-piperidine-2-yl-methanone
[4S-(3,5-bis-trifluoromethyl-benzyloxy)-3R-2-tolyl-piperidine-1-yl]-thiazolidin-4-yl-methanone
[4-(3,5-bis-trifluoromethyl-benzyloxy)-3R-2-tolyl-piperidine-1-yl]-(2-hydroxy-pyridine-3-yl)-methanone
[4-(3,5-bis-trifluoromethyl-benzyloxy)-3R-2-tolyl-piperidine-1-yl]-(3-hydroxy-pyridine-2-yl)-methanone
1-{2-[4S-(3,5-bis-trifluoromethyl-benzyloxy)-3R-2-tolyl-piperidine-1-carbonyl]-4R-hydroxy-pyrrolidine-1-yl)}-ethanone
[4S-(3,5-bis-trifluoromethyl-benzyloxy)-3R-2-tolyl-piperidine-1-yl]-pyridine-4-yl-methanone
1-{2-[4S-(3,5-bis-trifluoromethyl-benzyloxy)-3R-2-tolyl-piperidine-1-carbonyl]-pyrrolidin-1-yl)}-ethanone
[4S-(3,5-bis-trifluoromethyl-benzyloxy)-3R-2-tolyl-piperidine-1-yl]-pyrrolidin-1-yl-methanone
[4S-(3,5-bis-trifluoromethyl-benzyloxy)-3R-2-tolyl-piperidine-1-yl]-morpholin-4-yl-methanone
[4S-(3,5-bis-trifluoromethyl-benzyloxy)-3R-2-tolyl-piperidine-1-yl]-2-dimethylamino-ethanone
N-{2-[4S-(3,5-bis-trifluoromethyl-benzyloxy)-3R-2-tolyl-piperidine-1-yl]-2oxo-ethyl}-acetamide
2-amino-1-[4S-(3,5-bis-trifluoromethyl-benzyloxy)-3R-2-tolyl-piperidine-1-yl]-ethanone
2-[4S-(3,5-bis-trifluoromethyl-benzyloxy)-3R-2-tolyl-piperidine-1-yl]-N-N-dimethyl-acetamide
4-[4S-(3,5-bis-trifluoromethyl-benzyloxy)-3R-2-tolyl-piperidine-1-carbonyl]-piperidine-2,6-dione
[4S-(3,5-bis-trifluoromethyl-benzyloxy)-3R-2-tolyl-piperidine-1-yl]-pyrrolidin-2-yl-methanone
[4S-(3,5-bis-trifluoromethyl-benzyloxy)-3R-2-tolyl-piperidine-1-yl]-piperazine-2-yl-methanone
5-[4S-(3,5-bis-trifluoromethyl-benzyloxy)-3R-2-tolyl-piperidine-1-methyl]-2-4-dihydro-[1,2,4] triazol-3-one
5-[4S-(3,5-bis-trifluoromethyl-benzyloxy)-3R-(3,4-difluoro-phenyl)-2-piperidine-1-methyl]-2-4-dihydro-[1,2,4] triazol-3-one
5-[4S-(3,5-bis-trifluoromethyl-benzyloxy)-3R-phenyl-2-piperidine-1-methyl]-2-4-dihydro-[1,2,4] triazol-3-one
5-[4S-(3,5-dimethyl-benzyloxy)-3R-phenyl-2-piperidine-1-methyl]-2-4-dihydro-[1,2,4] triazol-3-one
5-[4-(3,5-bis-trifluoromethyl-benzyloxy)-3-(4-fluoro-phenyl)-2-piperidine-1-methyl]-2-4-dihydro-[1,2,4] triazol-3-one
5-[4-(3,5-bis-fluoromethyl-benzyloxy)-3-phenyl)-2-piperidine-1-methyl]-2-4-dihydro-[1,2,4] triazol-3-one
4-(3,5-bis-trifluoromethyl-benzloxy)-1-methyl-3-tolyl-piperidine
1-[4-(3,5-bis-trifluoromethyl-benzloxy)-3-(4-fluoro-2-methyl-phenyl)-piperidine-1-yl]-ethanone
1-[4S-(3,5-bis-trifluoromethyl-benzyloxy)-3R-2-tolyl-piperidine-1-yl]-ethanone
{5-[4-(3,5-bis-trifluoromethyl-benzloxy)-3-phenyl-piperidine-1-yl]-2H[1,2,3] triazol-4-methyl}dimethyl-amine
{5-[4-(3,5-bis-trifluoromethyl-benzloxy)-3-(4-fluoro-phenyl)-piperidine-1-yl-]-2H[1,2,3] triazol-4-methyl} dimethyl-amine
{5-[4-(3,5-dimethyl-benzloxy)-3-phenyl-piperidine-1-yl]-2H[1,2,3] triazol-4-methyl}dimethyl-amine

6. A compound according to claim 4 selected from the group consisting of:
4S-(3,5-bis-trifluoromethyl-benzyloxy)-3R-2-tolyl-piperidine
4-(3,5-bis-trifluoromethyl-benzyloxy)-3-phenyl-piperidine
4-(3,5-bis-trifluoromethyl-benzyloxy)-3-(4-fluoro-phenyl)-piperidine
4-(3,5-bis-trifluoromethyl-benzyloxy)-3-(2-methyl-4-fluoro-phenyl)-piperidine
4-(3,5-bis-trifluoromethyl-benzyloxy)-3-(3,4-difluoro-phenyl)-piperidine
4-(3,5-bis-trifluoromethyl-benzyloxy)-3-(2-pyridyl)-piperidine
4-(3,5-bis-trifluoromethyl-benzyloxy)-3-(3-chloro-phenyl)-piperidine
1-[4S-(3,5-bis-trifluoromethyl-benzyloxy)-3R-2-tolyl-piperidine-1-yl]-2-morpholin-1-yl-ethanone
1-[4S-(3,5-bis-trifluoromethyl-benzyloxy)-3R-2-tolyl-piperidine-1-yl]-2-piperazine-1-yl-ethanone
1-[4S-(3,5-bis-trifluoromethyl-benzyloxy)-3R-2-tolyl-piperidine-1-yl]-2-(4-methyl-piperazine-1-yl)-ethanone
1-[4S-(3,5-bis-trifluoromethyl-benzyloxy)-3R-2-tolyl-piperidine-1-yl]-2-(4-ethyl-piperazine-1-yl)-ethanone
1-[4S-(3,5-bis-trifluoromethyl-benzyloxy)-3R-2-tolyl-piperidine-1-yl]-2-(4-benzyl-piperazine-1-yl)-ethanone
1-[4S-(3,5-bis-trifluoromethyl-benzyloxy)-3R-2-tolyl-piperidine-1-yl]-2-piperidine-1-carboxylic acid tert-butyl ester-1-yl-ethanone
1-[4S-(3,5-bis-trifluoromethyl-benzyloxy)-3R-2-tolyl-piperidine-1-yl]-2-piperidine-4-yl-ethanone
1-[4S-(3,5-bis-trifluoromethyl-benzyloxy)-3R-2-tolyl-piperidine-1-yl]-2-(1-acetyl-piperidine-4-yl)-ethanone
1-[4S-(3,5-bis-trifluoromethyl-benzyloxy)-3R-2-tolyl-piperidine-1-yl]-2-(1-H-imidazole-4-yl)-ethanone
1-[4S-(3,5-bis-trifluoromethyl-benzyloxy)-3R-2-tolyl-piperidine-1-yl]-2-pyridine-4-yl-ethanone
1-[4S-(3,5-bis-trifluoromethyl-benzyloxy)-3R-2-tolyl-piperidine-1-yl]-2-pyrrolidin-2-one-1-yl-ethanone
1-[4S-(3,5-bis-trifluoromethyl-benzyloxy)-3R-2-tolyl-piperidine-1-yl]-2-dimethylamino-1-yl-ethanone
[4S-(3,5-bis-trifluoromethyl-benzyloxy)-3R-2-tolyl-piperidine-1-yl]-(4-methyl-piperazine-1-yl)-methanone
[4S-(3,5-bis-trifluoromethyl-benzyloxy)-3R-2-tolyl-piperidine-1-yl]-piperidine-4-yl-methanone
[4S-(3,5-bis-trifluoromethyl-benzyloxy)-3R-2-tolyl-piperidine-1-yl]-piperidine-2-yl-methanone
[4S-(3,5-bis-trifluoromethyl-benzyloxy)-3R-2-tolyl-piperidine-1-yl]-pyrrolidin-1-yl-methanone
[4S-(3,5-bis-trifluoromethyl-benzyloxy)-3R-2-tolyl-piperidine-1-yl]-morpholin-4-yl-methanone
[4S-(3,5-bis-trifluoromethyl-benzyloxy)-3R-2-tolyl-piperidine-1-yl]-2-dimethylamino-ethanone
2-[4S-(3,5-bis-trifluoromethyl-benzyloxy)-3R-2-tolyl-piperidine-1-yl]-N-N-dimethyl-acetamide
5-[4S-(3,5-bis-trifluoromethyl-benzyloxy)-3R-2-tolyl-piperidine-1-methyl]-2-4-dihydro-[1,2,4] triazol-3-one
5-[4S-(3,5-bis-trifluoromethyl-benzyloxy)-3R-(3,4-difluoro-phenyl)-2-piperidine-1-methyl]-2-4-dihydro-[1,2,4] triazol-3-one
5-[4S-(3,5-bis-trifluoromethyl-benzyloxy)-3R-phenyl-2-piperidine-1-methyl]-2-4-dihydro-[1,2,4] triazol-3-one
5-[4-(3,5-bis-trifluoromethyl-benzyloxy)-3-(4-fluoro-phenyl)-2-piperidine-1-methyl]-2-4-dihydro-[1,2,4] triazol-3-one
1-[4S-(3,5-bis-trifluoromethyl-benzyloxy)-3R-2-tolyl-piperidine-1-yl]-ethanone
and pharmaceutically acceptable salts or solvates thereof.

7. A compound according to claim 4 selected from the group consisting of:
3-(4-fluoro-2-methyl-phenyl)-4-(2-phenyl-butyrylamino)-piperidine
4-oxo-2,4-diphenyl-(3-phenyl-piperidin-4-yl)-butyramide
2-(4-nitro-phenyl) -(3-phenyl-piperidin-4-yl)-propionamide
2-phenyl-(3-phenyl-piperidin-4-yl)-propionamide
1,2,3,4-tetrahydro-naphthalene-1-carboxylic acid (3-phenyl-piperidin-4-yl)-amide:
3-methyl-2-phenyl-(3-phenyl-piperidin-4-yl)-butyramide
2-phenyl-(3-phenyl-piperidin-4-yl)-butyramide
2-(3-benzoyl-phenyl)-(3-phenyl-piperidin-4-yl)-propionamide
(3-phenyl-piperidin-4-yl)-2-tolyl-acetamide
(3-phenyl-piperidin-4-yl)-2-[4-(thiophene-2-carbonyl)-phenyl]-propionamide
6-fluoro-2-oxo-1,2,3,4-tetrahydro-quinoline-4-carboxylic acid (3-phenyl-piperidin-4-yl)-amide
3-furan-2-yl-2-phenyl-(3-phenyl-piperidin-4-yl)-propionamide
6-chloro-8-methyl-chroman-4-carboxylic acid (3-phenyl-piperidin-4-yl)-amide
5-cyclohexyl-indan-1-carboxylic acid (3-phenyl-piperidin-4-yl)-amide
2-(3,4-dimethoxy-phenyl)-hexanoic acid (3-phenyl-piperidin-4-yl)-amide
6-methyl-indan-1-carboxylic acid (3-phenyl-piperidin-4-yl)-amide
2-[3-chloro-4-(2,5-dihydro-pyrrol-1-yl)-phenyl-(3-phenyl-piperidin-4-yl)-propionamide
6-methoxy-3-oxo-indan-1-carboxylic acid (3-phenyl-piperidin-4-yl)-amide
6,7-dichloro-chroman-4-carboxylic acid (3-phenyl-piperidin-4-yl)-amide
2-{4-[2-(4-methoxy-phenyl)-vinyl]-phenyl-(3-phenyl-piperidin-4-yl)}-propionamide:
2-phenyl-(3-phenyl-piperidin-4-yl)-propionamide
2-(4-chloro-phenyl-3-phenyl-piperidin-4-yl)-propionamide
2-phenyl-hexanoic acid (3-phenyl-piperidin-4-yl)-amide
thiochroman-4-carboxylic acid (3-phenyl-piperidin-4-yl)-amide
5-methoxy-1,2,3,4-tetrahydro-naphthalene-1-carboxylic acid (3-phenyl-piperidin-4-yl)-amide
1-oxo-3-phenyl-1,2,3,4-tetrahydro-isoquinoline-4-carboxylic acid (3-phenyl-piperidin-4-yl)-amide
6-methoxy-2-methyl-1,2,3,4-tetrahydro-isoquinoline-4-carboxylic acid (3-phenyl-piperidin-4-yl)-amide
2-(4-hydroxy-phenyl)-3-phenyl-3-phenyl-piperidin-4-yl)-propionamide:
2-(2-fluoro-biphenyl-4-yl-3-phenyl-piperidin-4-yl)-propionamide
chroman-2-carboxylic acid (3-phenyl-piperidin-4-yl)-amide
2,4-diphenyl-3-phenyl-piperidin-4-yl)-butyramide
6-chloro-thiochroman-4-carboxylic acid (3-phenyl-piperidin-4-yl)-amide
7-methoxy-chroman-4-carboxylic acid (3-phenyl-piperidin-4-yl)-amide
2-[4-(2-hydroxy-2-methyl-propyl)-phenyl]-(3-phenyl-piperidin-4-yl)-propionamide:
2-phenyl-(3-phenyl-piperidin-4-yl)-butyramide
2-(4-hydroxy-phenyl-3-phenyl-piperidin-4-yl)-propionamide
2-phenyl-3-phenyl-piperidin-4-yl)-acetamide
2,3-diphenyl-(3-phenyl-piperidin-4-yl)-propionamide
2-(3-phenoxy-phenyl-3-phenyl-piperidin-4-yl)-propionamide
2-(4-isobutyl-phenyl-3-phenyl-piperidin-4-yl)-propionamide
2-phenyl-3-phenyl-piperidin-4-yl)-propionamide
indan-1-carboxylic acid (3-phenyl-piperidin-4-yl)-amide
2-phenoxy -(3-phenyl-piperidin-4-yl)-propionamide
3-(4-methoxy-phenyl)-2-phenyl-(3-phenyl-piperidin-4-yl)-propionamide
2-cyclopentyl-2-phenyl-(3-phenyl-piperidin-4-yl)-acetamide
1,2,3,4-tetrahydro-isoquinoline-4-carboxylic acid (3-phenyl-piperidin-4-yl)-amide
2-phenyl-3-(5-phenyl-furan-2-yl-3-phenyl-piperidin-4-yl)-propionamide
3-(4-hydroxy-phenyl)-2-phenyl-3-phenyl-piperidin-4-yl)-propionamide
6,7-dichloro-thiochroman-4-carboxylic acid (3-phenyl-piperidin-4-yl)-amide
6-fluoro-3-hydroxy-indan-1-carboxylic acid (3-phenyl-piperidin-4-yl)-amide
4,5,6,7-tetramethyl-3-oxo-indan-1-carboxylic acid (3-phenyl-piperidin-4-yl)-amide
2-(2,5-dimethyl-phenyl)-6-phenyl-hexanoic acid (3-phenyl-piperidin-4-yl)-amide
3-methyl-2-phenyl-pentanoic acid (3-phenyl-piperidin-4-yl)-amide
(3-phenyl-piperidin-4-yl)-2-tolyl-butyramide
6-fluoro-chroman-4-carboxylic acid (3-phenyl-piperidin-4-yl)-amide
7-methoxy-1,2,3,4-tetrahydro-naphthalene-1-carboxylic acid (3-phenyl-piperidin-4-yl)-amide
3-oxo-indan-1-carboxylic acid (3-phenyl-piperidin-4-yl)-amide
2-biphenyl-4-yl-pent-4-enoic acid (3-phenyl-piperidin-4-yl)-amide
2-naphthalen-1-yl-heptanoic acid (3-phenyl-piperidin-4-yl)-amide
2-(6-methoxy-naphthalen-2-yl-3-phenyl-piperidin-4-yl)-propionamide
2-(4-chloro-phenyl)-3-methyl-3-phenyl-piperidin-4-yl)-butyramide
5-methyl-2-tolyl-hexanoic acid (3-phenyl-piperidin-4-yl)-amide
6-methoxy-1,2,3,4-tetrahydro-naphthalene-1-carboxylic acid (3-phenyl-piperidin-4-yl)-amide
6-methoxy-chroman-4-carboxylic acid (3-phenyl-piperidin-4-yl)-amide
6-fluoro-3-oxo-indan-1-carboxylic acid (3-phenyl-piperidin-4-yl)-amide
3-hydroxy-2-phenyl-(3-phenyl-piperidin-4-yl)-propionamide
4-(4-methoxy-phenyl)-4-oxo-2-phenyl-(3-phenyl-piperidin-4-yl)-butyramide
6-chloro-9-methyl-2,3,4,9-tetrahydro-1-carbazole-4-carboxylic acid (3-phenyl-piperidin-4-yl)-amide
2-(4-isobutyl-phenyl-3-phenyl-piperidin-4-yl)-propionamide
2-phenoxy-3-phenyl-piperidin-4-yl)-butyramide
2-biphenyl-4-yl-hex-4-enoic acid (3-phenyl-piperidin-4-yl)-amide
2-cyclohex-2-enyl-2-phenyl-3-phenyl-piperidin-4-yl)-acetamide
6,7-dimethyl-chroman-4-carboxylic acid (3-phenyl-piperidin-4-yl)-amide
2-[2-(4-chloro-phenyl)-benzooxazol-5-yl-3-phenyl-piperidin-4-yl]-propionamide
3-(4-hydroxy-3,5-diiodo-phenyl)-2-phenyl-3-phenyl-piperidin-4-yl)-propionamide
2-(4-methoxy-phenyl)-3-(5-phenyl-furan-2-yl)-(3-phenyl-piperidin-4-yl)-propionamide
6-chloro-chroman-4-carboxylic acid (3-phenyl-piperidin-4-yl)-amide
4,5-dimethoxy-indan-1-carboxylic acid (3-phenyl-piperidin-4-yl)-amide
6,7-dichloro-2-methyl-chroman-4-carboxylic acid (3-phenyl-piperidin-4-yl)-amide
2-(6-hydroxy-naphthalen-2-yl-3-phenyl-piperidin-4-yl)-propionamide
and pharmaceutically acceptable salts and solvates of thereof.

8. A compound according to claim 4 selected from the group consisting of:
3-(4-fluoro-2-methyl-phenyl)-4-(2-phenyl-butyrylamino)-piperidine
2-phenyl-(3-phenyl-piperidin-4-yl)-propionamide
2,4-diphenyl-3-phenyl-piperidin-4-yl)-butyramide
2-phenyl-(3-phenyl-piperidin-4-yl)-butyramide
2-phenyl-3-phenyl-piperidin-4-yl)-acetamide
2,3-diphenyl-(3-phenyl-piperidin-4-yl)-propionamide
2-(phenyl-3-phenyl-piperidin-4-yl)-propionamide
(3-phenyl-piperidin-4-yl)-2-tolyl-butyramide
and pharmaceutically acceptable salts and solvates thereof.

9. A pharmaceutical composition for antagonizing the effect of NK-1 and/or NK-3 at their receptor sites in a mammal, comprising an NK-1 and/or NK-3 receptor antagonizing amount of a compound according to claim 1, or a pharmaceutically acceptable salt or solvate thereof, and a pharmaceutically acceptable carrier.

10. A pharmaceutical composition for treating a condition or disorder associated with the activity of NK-1 and/or NK-3 receptors in a mammal, comprising an amount of a compound according to claim 1, or a pharmaceutically acceptable salt or solvate thereof, and a pharmaceutically acceptable carrier, wherein the amount of said compound is effective in (1) antagonizing the NK-1 and/or NK-3 receptor, and/or (2) treating said condition or disorder.

11. A pharmaceutical composition for treating in a mammal a condition or disorder selected from the group consisting of sleep disorders, autism, pervasive development disorder, rheumatoid arthritis, osteoarthritis, fibromyalgia, human immunodeficiency virus (HIV) infections, dissociative disorders, anorexia, bulimia; ulcerative colitis, Crohn's disease, irritable bowel syndrome, functional abdominal pain, chronic fatigue syndrome, sudden infant death syndrome (SIDS), overactive bladder, chronic cystitis, chemotherapy induced cystitis, cough, angiotensin converting enzyme (ACE) induced cough, itch, hiccups, premenstrual syndrome, premenstrual dysphoric disorder, schizophrenia, schizoaffective disorder, delusional disorder, substance-induced psychotic disorder, brief psychotic disorder, shared psychotic disorder, psychotic disorder due to a general medical condition, schizophreniform disorder, amenorrheic disorders such as desmenorrhea, obesity, epilepsy, primary movement disorders, spasticities, Scott's syndrome, Tourette's syndrome, palsys, amyolateral sclerosis (ALS), akinetic-rigid disorders, akinesias, dyskinesias, restless leg syndrome, movement disorders associated with Parkinson's disease or Huntington's disease, mastalgia syndromes, motion sickness, immune dysfunctions, generalized anxiety disorder, panic disorder, social phobia, agoraphobia, specific phobias, obsessive-compulsive disorder, post-traumatic stress disorder, major depression, single episode depression, recurrent depression, child abuse induced depression, postpartum depression, dysthemia, cyclothymia, bipolar disorder, neurocardiac syncope, neurogenic syncope, hypersensitive Carotid sinus, neurovascular syndrome, arrythmias, addiction disorders involving addictions to behaviors, HIV-1 associated dementia, AIDS dementia complex, HIV encephalopathy, HIV related neuralgias, AIDS related neuralgias, epilepsy, attention deficit hyperactivity disorder, a somatoform disorder selected from the group consisting of somitization disorder, hypochondriasis, somatoform pain disorder and undifferentiated somatoform disorder, and somatic symptoms selected from the group consisting of loss of appetite, insomnia, interrupted sleep, early morning awakening, tired awakening, loss of libido, restlessness, fatigue, constipation, dyspepsia, heart palpitations, headache, neck pain, back pain, limb pain, joint pain, abdominal pain, dizziness, nausea, heartburn, nervousness, tremors, burning and tingling sensations, morning stiffness, abdominal pain, abdominal distention, gurgling, diarrhea, and the symptoms associated with generalized anxiety disorder, comprising an amount of a compound of Formula I, or a pharmaceutically acceptable salt or solvate thereof, and a pharmaceutically acceptable carrier, wherein the amount of said compound is effective in (1) antagonizing an NK-1 and/or NK-3 receptor, and/or (2) treating said condition or disorder.

12. A method of antagonizing an NK-1 and/or or NK-3 receptors in a mammal, comprising administering to said mammal an NK-1 or NK-3 antagonizing amount of a compound according to claim 1, or a pharmaceutically acceptable salt or solvate thereof.

13. A method of treating a condition or disorder associated with the activity of NK-1 and/or NK-3 receptors in a mammal, comprising administering to said mammal in need of said treatment an amount of a compound according to claim 1, or a pharmaceutically acceptable salt or solvate thereof, wherein the amount of said compound is effective in (1) antagonizing the NK-1 and/or NK-3 receptor, and/or (2) treating said condition or disorder.

14. A method of treating in a mammal a condition or disorder selected from the group consisting of sleep disorders, autism, pervasive development disorder, rheumatoid arthritis, osteoarthritis, fibromyalgia, human immunodeficiency virus (HIV) infections, dissociative disorders, anorexia, bulimia; ulcerative colitis, Crohn's disease, irritable bowel syndrome, functional abdominal pain, chronic fatigue syndrome, sudden infant death syndrome (SIDS), overactive bladder, chronic cystitis, chemotherapy induced cystitis, cough, angiotensin converting enzyme (ACE) induced cough, itch, hiccups, premenstrual syndrome, premenstrual dysphoric disorder, schizophrenia, schizoaffective disorder, delusional disorder, substance-induced psychotic disorder, brief psychotic disorder, shared psychotic disorder, psychotic disorder due to a general medical condition, schizophreniform disorder, amenorrheic disorders such as desmenorrhea, obesity, epilepsy, primary movement disorders, spasticities, Scott's syndrome, Tourette's syndrome, palsys, amyolateral sclerosis (ALS), akinetic-rigid disorders, akinesias, dyskinesias, restless leg syndrome, movement disorders associated with Parkinson's disease or Huntington's disease, mastalgia syndromes, motion sickness, immune dysfunctions, generalized anxiety disorder, panic disorder, social phobia, agoraphobia, specific phobias, obsessive-compulsive disorder, post-traumatic stress disorder, major depression, single episode depression, recurrent depression, child abuse induced depression, postpartum depression, dysthemia, cyclothymia, bipolar disorder, neurocardiac syncope, neurogenic syncope, hypersensitive Carotid sinus, neurovascular syndrome, arrythmias, addiction disorders involving addictions to behaviors, HIV-1 associated dementia, AIDS dementia complex, HIV encephalopathy, HIV related neuralgias, AIDS related neuralgias, epilepsy, attention deficit hyperactivity disorder, a somatoform disorder selected from the group consisting of somitization disorder, hypochondriasis, somatoform pain disorder and undifferentiated somatoform disorder, and somatic symptoms selected from the group consisting of loss of appetite, insomnia, interrupted sleep, early morning awakening, tired awakening, loss of libido, restlessness, fatigue, constipation, dyspepsia, heart palpitations, headache, neck pain, back pain, limb pain, joint pain, abdominal pain, dizziness, nausea, heartburn, nervousness, tremors, burning and tingling sensations, morning stiffness, abdominal pain, abdominal distention, gurgling, diarrhea, and the symptoms associated with generalized anxiety disorder, comprising administering to said mammal in need of said treatment an amount of a compound according to claim 1, or a pharmaceutically acceptable salt or solvate thereof, wherein the amount of said compound is effective in (1) antagonizing an NK-1 and/or NK-3 receptor, and/or (2) treating said condition or disorder.

15. The pharmaceutical composition according to claim 11, wherein said composition is formulated for oral or injectable administration.

16. The pharmaceutical composition according to claim 11, wherein said composition is an immediate release or a controlled release dosage form.

17. The compound according to claim 1, wherein in an assay of NK-1 binding, said compound exhibits a Ki of about 5 nM or less.

18. The compound according to claim 1, wherein in an assay of NK-3 binding, said compound exhibits a Ki of about 5 nM or less.

19. The method according to claim 14, wherein the compound that is employed is the compound according to claim 4

20. The method according to claim 14, wherein the disorder or condition being treated is cyclothymia or bipolar disorder.

21. The method according to claim 14, wherein the disorder or condition being treated is an addiction to a behavior.

22. The method according to claim 14, wherein the disorder or condition being treated is a sleep disorder.

23. The method according to claim 14, wherein the disorder or condition being treated is premenstrual syndrome or premenstrual dysphoric disorder.

24. The method according to claim 14, wherein the disorder or condition being treated is autism or pervasive development disorder.

25. The method according to claim 14, wherein the disorder or condition being treated is Scott's syndrome or Tourette's syndrome.

26. The method according to claim 14, wherein the disorder or condition being treated is obsessive-compulsive disorder.

27. The method according to claim 14, wherein the disorder or condition being treated is selected from the group consisting of primary movement disorders, spasticities, Scott's syndrome, Tourette's syndrome, palsys, amyolateral sclerosis (ALS), akinetic-rigid disorders, akinesias, dyskinesias, restless leg syndrome and movement disorders associated with Parkinson's disease or Huntington's disease.

28. The method according to claim 14, wherein the disorder or condition being treated is major depressive disorder.

29. The method according to claim 14, wherein the disorder or condition being treated is generalized anxiety disorder.

30. The method according to claim 14, wherein the disorder or condition being treated is irritable bowel syndrome.

31. The method according to claim 14, wherein the disorder or condition being treated is functional abdominal pain.

32. The method according to claim 14, wherein the disorder or condition being treated is an HIV infection.

33. The method according to claim 14, wherein the disorder or condition being treated is an immune dysfunction.

34. The method according to claim 14, wherein the disorder or condition being treated is selected from the group consisting of neurocardiac syncope, neurogenic syncope, hypersensitive Carotid sinus, neurovascular syndrome and arrythmias.

35. The method according to claim 14, wherein the disorder or condition being treated is selected from the group consisting of major depression, single episode depression, recurrent depression, child abuse induced depression, postpartum depression, dysthymia, cyclothymia and bipolar disorder.

36. The method according to claim 14, wherein the disorder or condition being treated is body dysmorphic disorders or eating disorders.

37. The method according to claim 14, wherein the disorder or condition being treated is selected from the group consisting of schizophrenia, schizoaffective disorder, delusional disorder, substance-induced psychotic disorder, brief psychotic disorder, shared psychotic disorder, psychotic disorder due to a general medical condition, and schizophreniform disorder.

38. The method according to claim 14, wherein the disorder or condition being treated is selected from the group consisting of premenstrual syndrome, premenstrual dysphoric disorder, and amenorrheic disorders.

39. The method according to claim 14, wherein the disorder or condition being treated is selected from the group consisting of Crohn's disease, ulcerative colitis, irritable bowel syndrome and functional abdominal pain.

40. The method according to claim 14, wherein the disorder or condition being treated is selected from the group consisting of autism, pervasive development disorder, and attention deficit hyperactivity disorder.

41. The method according to claim 14, wherein the disorder or condition being treated is selected from the group consisting of chronic fatigue syndrome, sudden infant death syndrome (SIDS), obesity, and epilepsy.

42. The method according to claim 14, wherein the disorder or condition being treated is selected from the group consisting of generalized anxiety disorder, panic disorder, obsessive-compulsive disorder, post-traumatic stress disorder, social phobia, agoraphobia, and specific phobias.

43. The method according to claim 14, wherein the disorder or condition being treated is selected from the group consisting of cough, angiotensin converting enzyme (ACE) induced cough, itch, and hiccups.

44. The method according to claim 14, wherein the disorder or condition being treated is selected from the group consisting of overactive bladder; chronic cystitis and chemotherapy induced cystitis.

45. The method according to claim 14, wherein the disorder or condition being treated is a sleep disorder.

46. The method according to claim 14, wherein the compound of Formula I is administered to a human for the treatment of major depressive disorder and concomitant neuropathic pain.

47. The method according to claim 14, wherein the compound of Formula I is administered to a human for the treatment of major depressive disorder and concomitant generalized anxiety disorder.

48. The method according to claim 14, wherein the disorder or condition being treated is fibromyalgia.

49. The method according to claim 14, wherein the disorder or condition being treated is AIDS related neuralgia.

50. The method according to claim 14, wherein the disorder or condition being treated is selected from the group consisting of schizophrenia, schizoaffective disorder, delusional disorder, substance-induced psychotic disorder, brief psychotic disorder, shared psychotic disorder, psychotic disorder due to a general medical condition, and schizophreniform disorder.

51. A method of treating a disorder or condition selected from the group consisting of pain resulting from soft tissue and peripheral damage, ostherpetic neuralgia, trigeminal neuralgia, segmental or intercostal neuralgia, pain associated with osteoarthritis, pain associated with rheumatoid arthritis, musculo-skeletal pain, spinal pain, dental pain, myofascial pain syndromes, episiotomy pain, pain resulting from burns, muscle pain, eye pain, orofacial pain, abdominal pain, gynaecological pain, labour pain, pain associated with endometriosis, pain associated with nerve and root damage, pain associated with nerve entrapment, pain associated with brachial plexus avulsions, pain associated with amputations, pain associated with peripheral neuropathies, atypical facial pain, pain associated with nerve root damage, neuropathic lower back pain, HIV related neuropathic pain, diabetic neuropathic pain, arachnoiditis, cancer pain, central nervous system pain, back pain; sciatica, phantom limb pain, migraine headaches, acute tension headache, chronic tension headache, cluster headache, temperomandibular pain, maxillary sinus pain, pain resulting from ankylosing spondylitis and gout; pain caused by increased bladder contractions, post operative pain, scar pain, chronic non-neuropathic, post surgical pain in a mammal, said method comprising administering to said mammal in need of said treatment an amount a compound according to claim 1, or a pharmaceutically acceptable salt or solvate thereof thereof, wherein the amount of said compound is effective in (1) antagonizing an NK-1 and/or NK-3 receptor, and/or (2) treating said condition or disorder.

52. The method according to claim 51, wherein the disorder or condition that is being treated is neuropathic pain.

53. The method according to claim 51, wherein the disorder or condition that is being treated is AIDS related neuralgia.

54. The method according to claim 51, wherein the disorder or condition that is being treated is pain associated with fibromyalgia.

55. The method according to claim 51, wherein the disorder or condition that is being treated is selected from the group consisting of neuropathic lower back pain, HIV related neuropathic pain, diabetic neuropathic pain, arachnoiditis and neuropathic and non-neuropathic pain associated with carcinoma.

56. The method according to claim 14, wherein the compound of Formula I is administered to a human for the treatment of major depressive disorder and concomitant premenstrual dysphoric disorder.

57. The method according to claim 14, wherein the compound of Formula I is administered to a human for the treatment of major depressive disorder and concomitant dysthymia.

58. The method according to claim 14, wherein the compound of Formula I is administered to a human for the treatment of major depressive disorder and concomitant fibromyalgia.

59. The method according to claim 14, wherein the compound of Formula I is administered to a human for the treatment of major depressive disorder and a concomitant somatoform disorder selected from the group consisting of somitization disorder, hypochondriasis, somatoform pain disorder and undifferentiated somatoform disorder.

60. The method according to claim 14, wherein the compound of Formula I is administered to a human for the treatment of generalized anxiety disorder and concomitant irritable bowel syndrome.

61. The method according to claim 14, wherein the compound of Formula I is administered to a human for the treatment of generalized anxiety disorder and concomitant functional abdominal pain.

62. The method according to claim 14, wherein the compound of Formula I is administered to a human for the treatment of generalized anxiety disorder and concomitant neuropathic pain.

63. The method according to claim 14, wherein the compound of Formula I is administered to a human for the treatment of generalized anxiety disorder and concomitant premenstrual dysphoric disorder.

64. The method according to claim 14, wherein the compound of Formula I is administered to a human for the treatment of generalized anxiety disorder and concomitant dysthymia.

65. The method according to claim 14, wherein the compound of Formula I is administered to a human for the treatment of generalized anxiety disorder and concomitant fibromyalgia.

66. The method according to claim 14, wherein the compound of Formula I is administered to a human for the treatment of generalized anxiety disorder and a concomitant somatoform disorder selected from the group consisting of somitization disorder, hypochondriasis, conversion disorder, body dysmorphic disorder, somatoform pain disorder and undifferentiated somatoform disorder.

67. The method according to claim 14, wherein the compound of Formula I is administered to a human for the treatment of major depressive disorder accompanied by one or more somatic symptoms selected from the group consisting of loss of appetite, sleep disturbances, loss of libido, restlessness, fatigue, constipation, dyspepsia, heart palpitations, aches and pains, dizziness, nausea, heartburn, nervousness, tremors, burning and tingling sensations, morning stiffness, abdominal symptoms, and the symptoms associated with generalized anxiety disorder.

68. The method according to claim 14, wherein the compound of Formula I is administered to a human for the treatment of major depressive disorder accompanied by one or more somatic symptoms selected from the group consisting of fatigue, headache, neck pain, back pain, limb pain, joint pain, abdominal pain, abdominal distention, gurgling, diarrhea nervousness, and the symptoms associated with generalized anxiety disorder.

69. The method according to claim 14, wherein the mammal being treated is a human who has not exhibited an adequate treatment response following treatment for the same disorder or condition with a selective serotonin reuptake inhibitor.

70. The method according to claim 14, wherein the mammal being treated is a human who has not exhibited an adequate treatment response following treatment for the same disorder or condition with a selective serotonin reuptake inhibitor.

71. The method according to claim 14, wherein the disorder or condition being treated is selected from the group consisting of HIV-1 associated dementia, AIDS dementia complex, HIV encephalopathy, and HIV related neuralgias.

72. The method according to claim 14, wherein the compound according to formula I is selected from the group consisting of:
4S-(3,5-bis-trifluoromethyl-benzloxy)-3R-2-tolyl-piperidine
4-(3,5-bis-trifluoromethyl-benzloxy)-3-phenyl-piperidine
4-(3,5-bis-trifluoromethyl-benzyloxy)-3-(4-fluoro-phenyl)-piperidine
4-(3,5-bis-trifluoromethyl-benzyloxy)-3-(2-methyl-4-fluoro-phenyl)-piperidine
4-(3,5-bis-trifluoromethyl-benzyloxy)-3-(3,4-difluoro-phenyl)-piperidine
4-(3,5-bis-trifluoromethyl-benzyloxy)-3-(2-pyridyl)-piperidine
4-(3,5-bis-trifluoromethyl-benzyloxy)-3-(3-chloro-phenyl)-piperidine
4-(4-bromo-benzyloxy)-3-(2-methyl-4-fluoro-phenyl)-piperidine
4-(3-bromo-benzyloxy)-3-(2-methyl-4-fluoro-phenyl)-piperidine
4-(2-bromo-benzyloxy)-3-(2-methyl-4-fluoro-phenyl)-piperidine
4-(2,6-dichloro-benzyloxy)-3-(2-methyl-4-fluoro-phenyl)-piperidine
4-(2,5-dichloro-benzyloxy)-3-(2-methyl-4-fluoro-phenyl)-piperidine
4-(3,5-dichloro-benzyloxy)-3-(2-methyl-4-fluoro-phenyl)-piperidine
4-(4-fluoro-benzyloxy)-3-(2-methyl-4-fluoro-phenyl)-piperidine
4-(2-fluoro-benzyloxy)-3-(2-methyl-4-fluoro-phenyl)-piperidine:
4-(3-fluoro-benzyloxy)-3-(2-methyl-4-fluoro-phenyl)-piperidine
4-(3,4-difluoro-benzyloxy)-3-(2-methyl-4-fluoro-phenyl)-piperidine
4-(3,4-difluoro-benzyloxy)-3-(2-methyl-4-fluoro-phenyl)-piperidine
4-(2,6-di-fluoro-benzyloxy)-3-(2-methyl-4-fluoro-phenyl)-piperidine
4-(3,6-di-fluoro-benzyloxy)-3-(2-methyl-4-fluoro-phenyl)-piperidine
4-(2,4,6-tri-fluoro-benzyloxy)-3-(2-methyl-4-fluoro-phenyl)-piperidine
4-(2,3,6-tri-fluoro-benzyloxy)-3-(2-methyl-4-fluoro-phenyl)-piperidine
4-(4-trifluoromethyl-benzyloxy)-3-(2-methyl-4-fluoro-phenyl)-piperidine
4-(3-trifluoromethyl-benzyloxy)-3-(2-methyl-4-fluoro-phenyl)-piperidine
4-(3,5-bis-trifluoromethyl-benzyloxy)-3-(2-methyl-4-fluoro-phenyl)-piperidine
4-(2,4-bis-trifluoromethyl-benzyloxy)-3-(2-methyl-4-fluoro-phenyl)-piperidine
4-(4-trifluoromethoxyl-benzyloxy)-3-(2-methyl-4-fluoro-phenyl)-piperidine
4-(2-methyl-3,4-Bisfluoromethyl-benzyloxy)-3-(2-methyl-4-fluoro-phenyl)-piperidine
4-(2-methyl-3,5-bis-fluoromethyl-benzyloxy)-3-(2-methyl-4-fluoro-phenyl)-piperidine
4-(2-ethyl-3,5-bis-fluoromethyl-benzyloxy)-3-(2-methyl-4-fluoro-phenyl)-piperidine
4-(2-methyl-benzyloxy)-3-(2-methyl-4-fluoro-phenyl)-piperidine
4-(2-chloro-5-methoxy-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid methylester
4-(3-methoxy-6-bromo-benzyloxy)-3-(2-methyl-4-fluoro-phenyl)-piperidine
4-(3-iodo-4-chloro-benzyloxy)-3-(2-methyl-4-fluoro-phenyl)-piperidine
4-(biphenyl-3-ylmethoxy)-3-(2-methyl-4-fluoro-phenyl)-piperidine
4-[2-(4-fluoro-benzyl)-benzyloxy]-3-(4-fluoro-2-methyl-phenyl)-piperidine
4-(3-methyl-benzyloxy)-3-(2-methyl-4-fluoro-phenyl)-piperidine
4-(2-trifluoromethyl-benzyloxy)-3-(2-methyl-4-fluoro-phenyl)-piperidine
4-(3-4-dimethyl-benzyloxy)-3-(2-methyl-4-fluoro-phenyl)-piperidine
4-(3-methyl-5-fluoro-benzyloxy)-3-(2-methyl-4-fluoro-phenyl)-piperidine
4-(2-methyl-3-fluoro-benzyloxy)-3-(2-methyl-4-fluoro-phenyl)-piperidine
4-(2,6-dibromo-benzyloxy)-3-(2-methyl-4-fluoro-phenyl)-piperidine
4-(3-chloro-6-methyl-benzyloxy)-3-(2-methyl-4-fluoro-phenyl)-piperidine
4-(2-iodo-benzyloxy)-3-(2-methyl-4-fluoro-phenyl)-piperidine
4-(2-isopropyl-benzyloxy)-3-(2-methyl-4-fluoro-phenyl)-piperidine
4-(3-fluoro-5-methyl-benzyloxy)-3-(2-methyl-4-fluoro-phenyl)-piperidine
4-[3-(2-methyl-4-fluoro-phenyl)-piperidine-4-yloxymethyl]-3-methoxy-benzoic acid ethylester
4-benzyloxy-3-(2-methyl-4-fluoro-phenyl)-piperidine
3-[3-(2-methyl-4-fluoro-phenyl)]-piperidine-4-yloxymethyl-pyridine
3-[3-(2-methyl-4-fluoro-phenyl)]-piperidine-4-yloxymethyl-benzonitrile
1-[4S-(3,5-bis-trifluoromethyl-benzyloxy)-3R-2-tolyl-piperidine-1-yl]-2-(4-acetyl-piperazine-1-yl)-ethanone
1-[4S-(3,5-bis-trifluoromethyl-benzyloxy)-3R-2-tolyl-piperidine-1-yl]-2-pyrrolidin-1-yl-ethanone
1-[4-(2-ethyl-3,5-difluorobenzyloxy)-3-(4-fluoro-2-methyl-phenyl)-piperidine] 2-pyrrolidin-1-yl-ethanone
1-[4-(3,5-dichlorobenzyloxy)-3-(4-fluoro-2-methyl-phenyl)-piperidine] 2-pyrrolidin-1-yl-ethanone
1-[4-(3-methyl-5-fluorobenzyloxy)-3-(4-fluoro-2-methyl-phenyl)piperidine] 2-pyrrolidin-1-yl-ethanone
1-[4-(3-iodo-4-chlorobenzyloxy)-3-(4-fluoro-2-methyl-phenyl)piperidine] 2-pyrrolidin-1-yl-ethanone:
1-[4-(4,5-dichlorobenzyloxy)-3-(4-fluoro-2-methyl-phenyl)piperidine] 2-pyrrolidin-1-yl-ethanone
1-[4-(2-trifluoromethylbenzyloxy)-3-(4-fluoro-2-methyl-phenyl)piperidine] 2-pyrrolidin-1-yl-ethanone
1-[4-(5-iodobenzyloxy)-3-(4-fluoro-2-methyl-phenyl)piperidine] 2-pyrrolidin-1-yl-ethanone
1-[4-(biphenyl-2-ylmethoxy)-3-(4-fluoro-2-methyl-phenyl)piperidine] 2-pyrrolidin-1-yl-ethanone:
1-[4-(2-chloro-5-methoxybenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(2,5-dibromobenzyloxy)-3-(4-fluoro-2-methyl-phenyl)piperidine] 2-pyrrolidin-1-yl-ethanone
1-[4-(4-iodobenzyloxy)-3-(4-fluoro-2-methyl-phenyl)piperidine] 2-pyrrolidin-1-yl-ethanone
1-[4-(5-bromobenzyloxy)-3-(4-fluoro-2-methyl-phenyl)piperidine] 2-pyrrolidin-1-yl-ethanone
1-[4-(2-methyl-5-fluorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2-iodobenzyloxy)-3-(4-fluoro-2-methyl-phenyl)piperidine] 2-pyrrolidin-1-yl-ethanone
1-[4-(2-difluoromethoxybenzyloxy)-3-(4-fluoro-2-methyl-phenyl)piperidine] 2-pyrrolidin-1-yl-ethanone:
1-[4-(2-methyl-5-chlorobenzyloxy)-3-(4-fluoro-2-methyl-phenyl)piperidine] 2-pyrrolidin-1-yl-ethanone
1-[4-(3-methylbenzyloxy)-3-(4-fluoro-2-methyl-phenyl)piperidine] 2-pyrrolidin-1-yl-ethanone
1-[4-(2-methyl-3,5-difluorobenzyloxy)-3-(4-fluoro-2-methyl-phenyl)piperidine] 2-pyrrolidin-1-yl-ethanone
1-[4-(3-methoxybenzyloxy)-3-(4-fluoro-2-methyl-phenyl)piperidine] 2-pyrrolidin-1-yl-ethanone
1-[4-(3-chlorobenzyloxy)-3-(4-fluoro-2-methyl-phenyl)piperidine] 2-pyrrolidin-1-yl-ethanone
1-[4-(3,5-difluorobenzyloxy)-3-(4-fluoro-2-methyl-phenyl)piperidine] 2-pyrrolidin-1-yl-ethanone
1-[4-(2,5-dichlorobenzyloxy)-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(4-bromobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2-methyl-3,4-difluorobenzyloxy)-3-(4-fluoro-2-methyl-phenyl)piperidine] 2-pyrrolidin-1-yl-ethanone
1-[4-(2-chlorobenzyloxy)-3-(4-fluoro-2-methyl-phenyl)piperidine] 2-pyrrolidin-1-yl-ethanone
1-[4-(2-methyl-3-fluorobenzyloxy)-3-(4-fluoro-2-methyl-phenyl)piperidine] 2-pyrrolidin-1-yl-ethanone
1-[4-(4-trifluoromethoxybenzyloxy)-3-(4-fluoro-2-methyl-phenyl)piperidine] 2-pyrrolidin-1-yl-ethanone
1-[4-(4-trifluoromethylbenzyloxy)-3-(4-fluoro-2-methyl-phenyl)piperidine] 2-pyrrolidin-1-yl-ethanone
1-[4-(2-fluorobenzyloxy)-3-(4-fluoro-2-methyl-phenyl)piperidine] 2-pyrrolidin-1-yl-ethanone
1-[4-(3,6-difluorobenzyloxy)-3-(4-fluoro-2-methyl-phenyl)piperidine] 2-pyrrolidin-1-yl-ethanone
1-[4-(2-cyanobenzyloxy)-3-(4-fluoro-2-methyl-phenyl)piperidine] 2-pyrrolidin-1-yl-ethanone
1-[4-(2,4,6-trifluorobenzyloxy)-3-(4-fluoro-2-methyl-phenyl)piperidine] 2-pyrrolidin-1-yl-ethanone
1-[4-(3-cyanobenzyloxy)-3-(4-fluoro-2-methyl-phenyl)phenyl] 2-pyrrolidin-1-yl-ethanone
1-[4-(5-methyl-6-fluorobenzyloxy)-3-(4-fluoro-2-methyl-phenyl)piperidine] 2-pyrrolidin-1-yl-ethanone
1-[4-(4-fluorobenzyloxy)-3-(4-fluoro-2-methyl-phenyl)piperidine] 2-pyrrolidin-1-yl-ethanone
1-[4-(2,6-dichlorobenzyloxy)-3-(4-fluoro-2-methyl-phenyl)piperidine] 2-pyrrolidin-1-yl-ethanone
1-[4-(2,5,6-trifluorobenzyloxy)-3-(4-fluoro-2-methyl-phenyl)piperidine] 2-pyrrolidin-1-yl-ethanone
1-[4-(2,6-difluorobenzyloxy)-3-(4-fluoro-2-methyl-phenyl)piperidine] 2-pyrrolidin-1-yl-ethanone
1-[4-(benzyloxy)-3-(4-fluoro-2-methyl-phenyl)piperidine]-2-pyrrolidin-1-yl-ethanone
1-[(4-fluorophenoxy) benzyloxy-3-(4-fluoro-2-methyl-phenyl)piperidine] 2-pyrrolidin-1-yl-ethanone
1-[4-(2-bromobenzyloxy)-3-(4-fluoro-2-methyl-phenyl)piperidine] 2-pyrrolidin-1-yl-ethanone
1-[4-(3-trifluoromethylbenzyloxy)-3-(4-fluoro-2-methyl-phenyl)piperidine] 2-pyrrolidin-1-yl-ethanone
1-[(4-benzoyl-benzyloxy)-3-(4-fluoro-2-methyl-phenyl)piperidine] 2-pyrrolidin-1-yl-ethanone
1-[4S-(2,4-bis-trifluoromethyl-benzyloxy)-3R-2-tolyl-piperidine-1-yl]-2-piperidine-1-yl-ethanone
1-[4S-(3,5-bis-trifluoromethyl-benzyloxy)-3R-2-tolyl-piperidine-1-yl]-2-morpholin-1-yl-ethanone
1-[4S-(3,5-bis-trifluoromethyl-benzyloxy)-3R-2-tolyl-piperidine-1-yl]-2-piperazine-1-yl-ethanone
1-[4S-(3,5-bis-trifluoromethyl-benzyloxy)-3R-2-tolyl-piperidine-1-yl]-2-(4-methyl-piperazine-1-yl)-ethanone
1-[4S-(3,5-bis-trifluoromethyl-benzyloxy)-3R-2-tolyl-piperidine-1-yl]-2-(4-ethyl-piperazine-1-yl)-ethanone
1-[4S-(3,5-bis-trifluoromethyl-benzyloxy)-3R-2-tolyl-piperidine-1-yl]-2-(4-benzyl-piperazine-1-yl)-ethanone
1-[4S-(3,5-bis-trifluoromethyl-benzyloxy)-3R-2-tolyl-piperidine-1-yl]-2-piperidine-1-carboxylic acid tert-butyl ester-1-yl-ethanone
1-[4S-(3,5-bis-trifluoromethyl-benzyloxy)-3R-2-tolyl-piperidine-1-yl]-2-piperidine-4-yl-ethanone
1-[4S-(3,5-bis-trifluoromethyl-benzyloxy)-3R-2-tolyl-piperidine-1-yl]-2-(1-Acetyl-piperidine-4-yl)-ethanone
1-[4S-(3,5-bis-trifluoromethyl-benzyloxy)-3R-2-tolyl-piperidine-1-yl]-2-(1-H-imidazole-4-yl)-ethanone
1-[4S-(3,5-bis-trifluoromethyl-benzyloxy)-3R-2-tolyl-piperidine-1-yl]-2-Pyridine-4-yl-ethanone
1-[4S-(3,5-bis-trifluoromethyl-benzyloxy)-3R-2-tolyl-piperidine-1-yl]-2-pyrrolidin-2-one-1-yl-ethanone
1-[4S-(3,5-bis-trifluoromethyl-benzyloxy)-3R-2-tolyl-piperidine-1-yl]-2-dimethylamino-1-yl-ethanone
[4S-(3,5-bis-trifluoromethyl-benzyloxy)-3R-2-tolyl-piperidine-1-yl]-(4-methyl-piperazine-1-yl)-methanone
[4S-(3,5-bis-trifluoromethyl-benzyloxy)-3R-2-tolyl-piperidine-1-yl]-piperidine-4-yl-methanone
[4S-(3,5-bis-trifluoromethyl-benzyloxy)-3R-2-tolyl-piperidine-1-yl]-piperidine-2-yl-methanone
[4S-(3,5-bis-trifluoromethyl-benzyloxy)-3R-2-tolyl-piperidine-1-yl]-thiazolidin-4-yl-methanone
[4-(3,5-bis-trifluoromethyl-benzyloxy)-3R-2-tolyl-piperidine-1-yl]-(2-hydroxy-pyridine-3-yl)-methanone
[4-(3,5-bis-trifluoromethyl-benzyloxy)-3R-2-tolyl-piperidine-1-yl]-(3-hydroxy-pyridine-2-yl)-methanone
1-{2-[4S-(3,5-bis-trifluoromethyl-benzyloxy)-3R-2-tolyl-piperidine-1-carbonyl]-4R-hydroxy-pyrrolidine-1-yl)}-ethanone
[4S-(3,5-bis-trifluoromethyl-benzyloxy)-3R-2-tolyl-piperidine-1-yl]-pyridine-4-yl-methanone
1-{2-[4S-(3,5-bis-trifluoromethyl-benzyloxy)-3R-2-tolyl-piperidine-1-carbonyl]-pyrrolidin-1-yl)}-ethanone
[4S-(3,5-bis-trifluoromethyl-benzyloxy)-3R-2-tolyl-piperidine-1-yl]-pyrrolidin-1-yl-methanone
[4S-(3,5-bis-trifluoromethyl-benzyloxy)-3R-2-tolyl-piperidine-1-yl]-morpholin-4-yl-methanone
[4S-(3,5-bis-trifluoromethyl-benzyloxy)-3R-2-tolyl-piperidine-1-yl]-2-dimethylamino-ethanone
N-{2-[4S-(3,5-bis-trifluoromethyl-benzyloxy)-3R-2-tolyl-piperidine-1-yl]-2oxo-ethyl}-acetamide
2-amino-1-[4S-(3,5-bis-trifluoromethyl-benzyloxy)-3R-2-tolyl-piperidine-1-yl]-ethanone
2-[4S-(3,5-bis-trifluoromethyl-benzyloxy)-3R-2-tolyl-piperidine-1-yl]-N-N-dimethyl-acetamide
4-[4S-(3,5-bis-trifluoromethyl-benzyloxy)-3R-2-tolyl-piperidine-1-carbonyl]-piperidine-2,6-dione
[4S-(3,5-bis-trifluoromethyl-benzyloxy)-3R-2-tolyl-piperidine-1-yl]-pyrrolidin-2-yl-methanone
[4S-(3,5-bis-trifluoromethyl-benzyloxy)-3R-2-tolyl-piperidine-1-yl]-piperazine-2-yl-methanone
5-[4S-(3,5-bis-trifluoromethyl-benzyloxy)-3R-2-tolyl-piperidine-1-methyl]-2-4-dihydro-[1,2,4] triazol-3-one
5-[4S-(3,5-bis-trifluoromethyl-benzyloxy)-3R-(3,4-difluoro-phenyl)-2-piperidine-1-methyl]-2-4-dihydro-[1,2,4] triazol-3-one
5-[4S-(3,5-bis-trifluoromethyl-benzyloxy)-3R-phenyl-2-piperidine-1-methyl]-2-4-dihydro-[1,2,4] triazol-3-one
5-[4S-(3,5-dimethyl-benzyloxy)-3R-phenyl-2-piperidine-1-methyl]-2-4-dihydro-[1,2,4] triazol-3-one
5-[4-(3,5-bis-trifluoromethyl-benzyloxy)-3-(4-fluoro-phenyl)-2-piperidine-1-methyl]-2-4-dihydro-[1,2,4] triazol-3-one
5-[4-(3,5-bis-fluoromethyl-benzyloxy)-3-phenyl)-2-piperidine-1-methyl]-2-4-dihydro-[1,2,4] triazol-3-one
4-(3,5-bis-trifluoromethyl-benzloxy)-1-methyl-3-tolyl-piperidine
1-[4-(3,5-bis-trifluoromethyl-benzloxy)-3-(4-fluoro-2-methyl-phenyl)-piperidine-1-yl]-ethanone
1-[4S-(3,5-bis-trifluoromethyl-benzyloxy)-3R-2-tolyl-piperidine-1-yl]-ethanone
{5-[4-(3,5-bis-trifluoromethyl-benzloxy)-3-phenyl-piperidine-1-yl]-2H[1,2,3] triazol-4-methyl}dimethyl-amine
{5-[4-(3,5-bis-trifluoromethyl-benzyloxy)-3-(4-fluoro-phenyl)-piperidine-1-yl]-2H[1,2,3] triazol-4-methyl} dimethyl-amine
{5-[4-(3,5-dimethyl-benzloxy)-3-phenyl-piperidine-1-yl]-2H[1,2,3] triazol-4-methyl}dimethyl-amine
3-(4-fluoro-2-methyl-phenyl)-4-(2-phenyl-butyrylamino)-piperidine
4-oxo-2, 4-diphenyl-(3-phenyl-piperidin-4-yl)-butyramide
2-(4-nitro-phenyl) -(3-phenyl-piperidin-4-yl)-propionamide
2-phenyl-(3-phenyl-piperidin-4-yl)-propionamide 1,2,3,4-tetrahydro-naphthalene-1-carboxylic acid (3-phenyl-piperidin-4-yl)-amide:
3-methyl-2-phenyl-(3-phenyl-piperidin-4-yl)-butyramide
2-phenyl-(3-phenyl-piperidin-4-yl)-butyramide
2-(3-benzoyl-phenyl)-(3-phenyl-piperidin-4-yl)-propionamide
(3-phenyl-piperidin-4-yl)-2-tolyl-acetamide
(3-phenyl-piperidin-4-yl)-2-[4-(thiophene-2-carbonyl)-phenyl]-propionamide
6-fluoro-2-oxo-1,2,3,4-tetrahydro-quinoline-4-carboxylic acid (3-phenyl-piperidin-4-yl)-amide
3-furan-2-yl-2-phenyl-(3-phenyl-piperidin-4-yl)-propionamide
6-chloro-8-methyl-chroman-4-carboxylic acid (3-phenyl-piperidin-4-yl)-amide
5-cyclohexyl-indan-1-carboxylic acid (3-phenyl-piperidin-4-yl)-amide
2-(3,4-dimethoxy-phenyl)-hexanoic acid (3-phenyl-piperidin-4-yl)-amide
6-methyl-indan-1-carboxylic acid (3-phenyl-piperidin-4-yl)-amide
2-[3-chloro-4-(2,5-dihydro-pyrrol-1-yl)-phenyl-(3-phenyl-piperidin-4-yl)-propionamide
6-methoxy-3-oxo-indan-1-carboxylic acid (3-phenyl-piperidin-4-yl)-amide
6,7-dichloro-chroman-4-carboxylic acid (3-phenyl-piperidin-4-yl)-amide
2-{4-[2-(4-methoxy-phenyl)-vinyl]-phenyl-(3-phenyl-piperidin-4-yl)}-propionamide:
2-phenyl-(3-phenyl-piperidin-4-yl)-propionamide
2-(4-chloro-phenyl-3-phenyl-piperidin-4-yl)-propionamide
2-phenyl-hexanoic acid (3-phenyl-piperidin-4-yl)-amide
thiochroman-4-carboxylic acid (3-phenyl-piperidin-4-yl)-amide
5-methoxy-1,2,3,4-tetrahydro-naphthalene-1-carboxylic acid (3-phenyl-piperidin-4-yl)-amide
1-oxo-3-phenyl-1,2,3,4-tetrahydro-isoquinoline-4-carboxylic acid (3-phenyl-piperidin-4-yl)-amide
6-methoxy-2-methyl-1,2,3,4-tetrahydro-isoquinoline-4-carboxylic acid (3-phenyl-piperidin-4-yl)-amide
2-(4-hydroxy-phenyl)-3-phenyl-3-phenyl-piperidin-4-yl)-propionamide:
2-(2-fluoro-biphenyl-4-yl-3-phenyl-piperidin-4-yl)-propionamide
chroman-2-carboxylic acid (3-phenyl-piperidin-4-yl)-amide
2,4-diphenyl-3-phenyl-piperidin-4-yl)-butyramide
6-chloro-thiochroman-4-carboxylic acid (3-phenyl-piperidin-4-yl)-amide
7-methoxy-chroman-4-carboxylic acid (3-phenyl-piperidin-4-yl)-amide
2-[4-(2-hydroxy-2-methyl-propyl)-phenyl]-(3-phenyl-piperidin-4-yl)-propionamide:
2-phenyl-(3-phenyl-piperidin-4-yl)-butyramide
2-(4-hydroxy-phenyl-3-phenyl-piperidin-4-yl)-propionamide
2-phenyl-3-phenyl-piperidin-4-yl)-acetamide
2,3-diphenyl-(3-phenyl-piperidin-4-yl)-propionamide
2-(3-phenoxy-phenyl-3-phenyl-piperidin-4-yl)-propionamide
2-(4-isobutyl-phenyl-3-phenyl-piperidin-4-yl)-propionamide
2-phenyl-3-phenyl-piperidin-4-yl)-propionamide
indan-1-carboxylic acid (3-phenyl-piperidin-4-yl)-amide
2-phenoxy -(3-phenyl-piperidin-4-yl)-propionamide
3-(4-methoxy-phenyl)-2-phenyl-(3-phenyl-piperidin-4-yl)-propionamide
2-cyclopentyl-2-phenyl-(3-phenyl-piperidin-4-yl)-acetamide
1,2,3,4-tetrahydro-isoquinoline-4-carboxylic acid (3-phenyl-piperidin-4-yl)-amide
2-phenyl-3-(5-phenyl-furan-2-yl-3-phenyl-piperidin-4-yl)-propionamide
3-(4-hydroxy-phenyl)-2-phenyl-3-phenyl-piperidin-4-yl)-propionamide
6,7-dichloro-thiochroman-4-carboxylic acid (3-phenyl-piperidin-4-yl)-amide
6-fluoro-3-hydroxy-indan-1-carboxylic acid (3-phenyl-piperidin-4-yl)-amide
4,5,6,7-tetramethyl-3-oxo-indan-1-carboxylic acid (3-phenyl-piperidin-4-yl)-amide
2-(2,5-dimethyl-phenyl)-6-phenyl-hexanoic acid (3-phenyl-piperidin-4-yl)-amide
3-methyl-2-phenyl-pentanoic acid (3-phenyl-piperidin-4-yl)-amide
(3-phenyl-piperidin-4-yl)-2-tolyl-butyramide
6-fluoro-chroman-4-carboxylic acid (3-phenyl-piperidin-4-yl)-amide
7-methoxy-1,2,3,4-tetrahydro-naphthalene-1-carboxylic acid (3-phenyl-piperidin-4-yl)-amide
3-oxo-indan-1-carboxylic acid (3-phenyl-piperidin-4-yl)-amide
2-biphenyl-4-yl-pent-4-enoic acid (3-phenyl-piperidin-4-yl)-amide
2-naphthalen-1-yl-heptanoic acid (3-phenyl-piperidin-4-yl)-amide
2-(6-methoxy-naphthalen-2-yl-3-phenyl-piperidin-4-yl)-propionamide
2-(4-chloro-phenyl)-3-methyl-3-phenyl-piperidin-4-yl)-butyramide
5-methyl-2-tolyl-hexanoic acid (3-phenyl-piperidin-4-yl)-amide
6-methoxy-1,2,3,4-tetrahydro-naphthalene-1-carboxylic acid (3-phenyl-piperidin-4-yl)-amide
6-methoxy-chroman-4-carboxylic acid (3-phenyl-piperidin-4-yl)-amide
6-fluoro-3-oxo-indan-1-carboxylic acid (3-phenyl-piperidin-4-yl)-amide
3-hydroxy-2-phenyl-(3-phenyl-piperidin-4-yl)-propionamide
4-(4-methoxy-phenyl)-4-oxo-2-phenyl-(3-phenyl-piperidin-4-yl)-butyramide
6-chloro-9-methyl-2,3,4,9-tetrahydro-1-carbazole-4-carboxylic acid (3-phenyl-piperidin-4-yl)-amide
2-(4-isobutyl-phenyl-3-phenyl-piperidin-4-yl)-propionamide
2-phenoxy-3-phenyl-piperidin-4-yl)-butyramide
2-biphenyl-4-yl-hex-4-enoic acid (3-phenyl-piperidin-4-yl)-amide
2-cyclohex-2-enyl-2-phenyl-3-phenyl-piperidin-4-yl)-acetamide
6,7-dimethyl-chroman-4-carboxylic acid (3-phenyl-piperidin-4-yl)-amide
2-[2-(4-chloro-phenyl)-benzooxazol-5-yl-3-phenyl-piperidin-4-yl]-propionamide
3-(4-hydroxy-3,5-diiodo-phenyl)-2-phenyl-3-phenyl-piperidin-4-yl)-propionamide
2-(4-methoxy-phenyl)-3-(5-phenyl-furan-2-yl)-(3-phenyl-piperidin-4-yl)-propionamide
6-chloro-chroman-4-carboxylic acid (3-phenyl-piperidin-4-yl)-amide
4,5-dimethoxy-indan-1-carboxylic acid (3-phenyl-piperidin-4-yl)-amide
6,7-dichloro-2-methyl-chroman-4-carboxylic acid (3-phenyl-piperidin-4-yl)-amide
2-(6-hydroxy-naphthalen-2-yl-3-phenyl-piperidin-4-yl)-propionamide
and pharmaceutically acceptable salts or solvates thereof.
